SEQUENCE LISTING (86 sequences)

SEQ ID NO: 1 - DNA, 211 bp, Artificial Sequence
  synthetic construct; ryanodine receptor-like protein fragment
gataataagttcgattttttacgaaaatgacaagtatcgagactgtggggaccattgtcctgaaattgctgaagttggtgatcaatttgatatgtctcatcttgtaccgaaccggatatcaaggctacttcttgggagtaggaggaacctggaatctaaacgaagaaaaaaatcccgatgcagaaattgtggcttccggcgtattcgtagg

SEQ ID NO: 2 - DNA, 162 bp, Artificial Sequence
  synthetic construct; hypothetical protein fragment
tttgttatttttgttttgtttagtgttagcgttaaaggactagatggtgatcttccaccagatgtcgaaactatcgatgtggagactacgaatgtaagtaacgcggatccagacgcagggaaacctattccggtggaagatacgcctaatggagtccctcca

SEQ ID NO: 3 - DNA, 161 bp, Artificial Sequence
  synthetic construct; proteosome subunit alpha type 3 fragment
aatatggttcaaatattcctcttaaatacctaaatgatagagtaagcatgtacatgcatgcatacactttatacagtgctgttagaccatttggttgcagtgtcatcttggccagttatgaagatagtgacccatctatgtatctgattgatccatctgga

SEQ ID NO: 4 - DNA, 203 bp, Artificial Sequence
  synthetic construct; Proteosome subunit beta type 1 fragment
taccagagaacaaaacaaacttttcccactatcaggcactactgttttgggttgtgcaggatgttggtgtgacactctaacattaaccagaatccttaaatctcgcatgcagatgtaccaacaagagcataacaaaacaatgtctacaactgcatgtgcccagatgttgtcaaccatgctctactacaagagattctttcctt

SEQ ID NO: 5 - DNA, 557 bp, Artificial Sequence
  synthetic construct; ribosomal protein s10e fragment
ggggctttctgatttttgacagcttctatagaagtttatcaagatgttgatgccaaaaaagaatagagtatgtatttacgaatacctcttcaaagagggagtcatggtagctaaaaaagattaccatgccccaaaacacctcgaactagaaactatccctaaccttcaagtaattaaggctttacaatcacttaaatcaaaaggttacgtaaaggaacaattcgcctggaggcattattattggtatttgactaactctggcatcgaatacctccgcacattcttacacttacctggagaaattgtcccatctaccttgaaacgcccagcaaggacagaaaccacccgtcctagaccagctgctctcagatctgagacatctaaaccttcagaagaccgtgcaggatacagaaggactcctggaggccctggagctgacaagaaagctgatgttggtccaggaactggagatgttgagttcaggcaaggattcggacgtggacgggcaccacaataaatttattgataagttaatttttataaattgatcagccaat

SEQ ID NO: 6 - DNA, 474 bp, Spodoptera frugiperda
atggtgtcggtgcaaactatcgcgacgattgtcgtgaagacattcaaaattgtattgaacatagtaattctggttctttaccgaactggttacaatggagagttcttgggagtgggaggtacgtggaacctcaatgaggagaagaatccggacgctgagatcgtggcctctggagtcatcgtgggctacctcatctacacactcgtgcaaatgatcacattcctctttggtaccactgagcataagagggccatgtcagagatagtgatgaacttcgtgggtgtgttcatgtggatcgccgtgggtgccgtggcgctgcactactggggcggttaccagggagagcaccagtaccagttcgtgtttgctgagaaacaggtgggtattgctgtcggcgccctctgcgtgatcaacggcgcagtgtacctcctagacacggcactctccgtcatacatttcaccaaggagatgtaa

SEQ ID NO: 7 - DNA, 474 bp, Ostrinia nubilalis
atggtgtccgtacaaacgattgcgacgatagtggtgaaaaccttcaaagttgttctaaacatcattatcctggtgctgtaccgcacggggtataatggtgagttcctcggagtgggaggaacgtggaacctcaacgaggagaagaatccggacgctgaaatagttgcttctggagtcattgtgggatacctcatctacacattggtgcaaatcgtcacatacctcttcggcactacagagcataaaagagccctgtcagaggtggtgatgaacttcgttggtgtgttcatgtggctggctgtgggcgcggtggcgctgcactactggggaggataccagggagaacaccagttccagtttgtctttgcagagaaacaggtgggcattgccgtgggagcgctctgcgtcatccagggagcggtgtacctgctcgacacggccctctccgtcatacattacgcaaaggagatgtaa

SEQ ID NO: 8 - DNA, 474 bp, Helicoverpa zea
atggtgtccgtacaaaccatagcgacgatagtcgtgaaaacattcaaagttgtactgaacatagtgatcctagttctctaccgaacgggttacaatggcgagttcctcggagtgggcggcacctggaacctcaatgaggagaagaaccctgacgctgagattgtagcctctggagtcattgtcggctacctcatatacacgctcgtacaaatcgtcacgttcctgtttggcactactgagcataaaagagcgctgtcagagatagtgatgaacttcgtgggcgtgttcctgtggatcgcggtgggtgctgtcgcgctgcactactggggcggctaccagggagaacaccagtaccagttcgtgttcgctgagaaacaggttggtttggcagtgggcgccctctgcgtgatcaacggagcagtgtacttgctagacacggcgctatccgtcattcatttcaccaaggagatgtaa

SEQ ID NO: 9 - DNA, 480 bp, Phyllotreta cruciferae
atgacaagtatagagaccgtgggtaccatcgtacttaaattattgaagttggtaataaacttgatatgcataataatatacaggaccggttaccagggctacttcttgggagtaggtggaacttggaacttaaacgaagaaaaaaatcctgatgcggaaattgtggcatcaggaatttttgtcggctacatgatctacactaccgtatctcttataagtttctgtttcgccagcggtgatcacaaaacatctttcaccgatattttaatgaacatagtcggtgtgttcatgtgggtggccgttggagcaacagcacttcactactggcttggttatttgtcagaatataaatacacgaccgttgattccgagcgacaggtgggattggcgttaggagcgttatgcgttataaacggggcagtctatctagtggacacggtgttgtctgtagtatttttgataaaagctaagatgcagtaa

SEQ ID NO: 10 - DNA, 480 bp, Phyllotreta striolata
atgacaagtatagagacagtgggtaccatcgtacttaaaatattgaagttggtaataaacttgatatgcataatactatacaggaccggttaccagggctacttcttaggagtaggaggaacatggaacttaaacgaagaaaaaaatcctgattcggaaattgtggcatctggaatctttgtcggctacatgatctacactatcgtatctcttataagcctgtgtttcgccagcggtgatcacaaaacatctttcaccgatattttaatgaatatagtcggtgtattcatgtgggtggccgttggagcaacagcacttcactactggctcggttatttgtcagaatataaatactcgagcgttgattccgagcgacaggtgggcttggcgttaggagccttgtgcgttataaacggtgcagtctatctagtggacagtgtgttgtctgtcgtatttataattaaatcgaagatgcaataa

SEQ ID NO: 11 - DNA, 483 bp, Acyrthosiphon pisum
atggtcaacatagcaagcgtgggcacggtggtcatcaaagtcgtgaaattggtgttgaacataatcattttggtgctgtaccggaccgggtatcgcggtggatttttgggagtcggcggaacgtggaacctgaacgaagagaaaaacccggatgcggaaatagtcgcttccggcgtgttcgtcggtttcttcatctacaccgtcgtgattctaatatcgtacgggttcggatccaaccaccagaagaaaaccctagtggatataataatgaatttcgtcggcatgttcatgtttatcgccgtcggcggtatagcgctccactattggattggttatcagaacgaaaacaaatacatcagcgtaacttccgaacgtgccataggcatcacggtgggcgtgctgtgcgttatttccggtgcaatttacctggtggacaccgtactgtcgtttatacattttgccagagaaatggagttcgattaa

SEQ ID NO: 12 - DNA, 483 bp, Apis florea
atggtgtctctggaaactgtcgcgggtatagtgataaaagttctcaagttgattattaatctaataatcctgatcctgtatcgaacgggatacaagggagaattcctgggtgtcggagggacatggaacttgaacgaagacaaaaatccagacgcagaaatcatcgcatccggagttttggtcggatttttcatatacaccagcgtcgttttaatcacttactgctttgggagcatgtatcacaagaagacacttgtcgaaattataatgaactttgttggaaccttcatgttcatcgctgtggggggtacagcgcttcattattggcatggttatcaaccggaaaataaatacgattcgattgttcaagaacgacagatcggcttagctgttggagcgttatgcgttatcgagggcgcaatgtatctcgtcgacttaatattgagttttctccattttgcgaaaaataccgacaatttttag

SEQ ID NO: 13 - DNA, 489 bp, Apis mellifera
atggtgtctttggaaactgtcgcgggtatagtgataaaagttctcaagttgattattaatctgataatcctgatcctgtatcgaacgggatacaaaggagaattcctgggtgtcggagggacatggaacttaaacgaagataaaaatccagacgctgagatggttgcgtccggagttttggtcggatttttcatatacaccagcgtcgttctgatctcttactgcttcgggagcatgtatcacaagaagacacttgtcgaaattataatgaacttcgttggaacgtttatgttcatcgctgtggggggtacagcgcttcattattggcatggttatcaaccggaaaataaattcgacacgattgttcaagaacgacagatcggcttagctgttggagcgttatgcgttatcgagggtgcagcgtatctcgtcgatttaatattgagttttctccatttttcgaaaaataccgacaattttttagaataa

SEQ ID NO: 14 - DNA, 489 bp, Bombus impatiens
atggtgtctttagaaactattgcgggtatagcgataaaagtgcttaaactggttattaatcttataatcctgatcctgtatcgaacaggataccaaggggaattcctgggtgttggaggaacatggaacttgaacgaagacaaaaatccggacgcagaaatcgttgcgtctggagttttagtgggatttttcatatacaccagcgttgttctaattacttattgctttggcaattcgtctcacaaaaagacacttgttgaaattataatgaacttcgttggaaccttcatgttcatcgctgtaggtggcacagcactccattattggcacggatatcaatcagaaaataaattcgatacgattgtccaggaacgacaggtcggcctagctgttggatcgttgtgcgttatcgagggtgcagcgtatctcgtcgatctaatattaagcgttctacattttgcaaaaagccacgatgaatatttagaataa

SEQ ID NO: 15 - DNA, 489 bp, Bombus terrestris
atggtgtctttagaaactattgcgggtatagcgataaaagtgcttaaactggttattaatctcataatcctgatcctgtatcgaacaggataccaaggggaattcctgggtgttggaggaacatggaacttgaacgaagacaaaaatccggacgcagaaatcgttgcgtctggagttttagtgggatttttcatatacaccagcgttgttctaattacttattgctttggcaactcgtcgcacaaaaagacacttgttgaaattataatgaacttcgttggaaccttcatgttcattgctgtaggtggcacagcactccattattggcacggatatcaatcagaaaataaattcgatacgattgtccaggaacgacaggtcggcctagctgttggatcgttgtgcgttatcgagggtgcagcgtatctcgtcgatctaatattaagcgttctacattttgcgaaaagccacgatgaatatttagaataa

SEQ ID NO: 16 - DNA, 480 bp, Nasonia vitripennis
atggtgtccgtacagacgatcggcggcatcgtcgtcaaggggcttaaattgctactgaatttgatcatcctcattctctaccgaacgggctacagcggagaatttttgggcgtgggtggaacatggaacctgaacgaagaaaagaatccagacgcggaaatcgtcgcttccggtgttttcgtcggctttttcatctacaccagcgtcgtgcttatcagcttttgctttggcaccatggatcacaagaagtcgatcgttgaagtcatcatgaactttgtgggcatgttcatgttcctcgcagtcggcggcactgccctgcactactggcacggctacatgtcggagcacaagtacctgcacgttgcaacggagagacaggtcggcctggcgctgggatctctgtgcgtgctagaaggcgcgctgtacctcgtcgatctcatattgacgggtttgcacatcgccaaggaggagttcgcctaa

SEQ ID NO: 17 - DNA, 492 bp, Megachile rotundata
atggtgtctctggagactatcgcgagtatagtgatcaaagttctgaagctggtaattaatctcataatcctgatcctgtaccgaacaggatacaaggatggagagtttctgggtgtcggtggcacgtggaatttgaacgaagataaaaatccagatgcagaaatcgtggcttctggtgttctagtgggcttcttcatatacaccagtgttgtactcatcacttactgttttgggagcaccaatcacaaaaagacccttgttgaaatcctgatgaattttgttgggaccttcatgttcatcgcagtaggaggaacagccctccattactggaacggttatcaaccagaaaacaagtacgacacggttgtccaagaacgacaggttggcttagctgttggatccctctgcgtcatcgaaggctccgcgtacctcgttgatttaatacttagttttctacattttgcgaaaaatcgagatgaaatgttcgactaa

SEQ ID NO: 18 - DNA, 474 bp, Spodoptera exigua
atggtgtcggtgcaaactatcgcgacgattgtcgtgaagacattcaagattgtgttgaacatagtaattctggttctttaccgaactggttacaatggggagttcttgggagtgggaggtacgtggaacttgaacgaggagaagaatccagatgctgagatcgtggcttctggagtcatcgtgggctacctcatatacacacttgtacaaatgatcacattcctgtttggtaccactgagcataaaagggcaatgtcagagatagtgatgaacttcgtgggtgtgttcatgtggatcgccgtgggtgccgtagctctgcactactggggaggttaccagggagagcaccaataccagttcgtgtttgctgagaaacaggtgggtattgctgtcggtgccctctgcgtgatcaacggtgcagtgtatctcttagacacggcactatccgtcatacacttcacaaaggagatgtaa

SEQ ID NO: 19 - DNA, 477 bp, Chrysopa pallens
atggtgtcgatacaaactgttggtagtgttgtaataaaaagttttaaattgcttttaaacttaattatcttaatcttataccgaaccggatacgatggacaacttttgggtgtgggtggaacatggaacctaaatgaggataaaaatccagatgtggaaatagtagcaagtggtgtatttgttggatacttcatatatacatcagtaacattaattgcattctgttttggatccaaagaatataaacgagagccagttgatataattatgaattttgttggtgtattcatgtggttagctgttggtggagtagcaattcattattggtctggttatcaaaatgaaaatcacggtcaattagatggtggtgaacgtgcaaaaggtctggcagttggtattttatgtgtaatttcaggtgcaacgtatctattggatactgtgttagccgttttacattatattcgtgaaaatttatag

SEQ ID NO: 20 - DNA, 474 bp, Bombyx mori
atggtttccgttcagactatagcgactatagtcgtgaagacattcaaaattgtcttgaacattataatcttagtgctctaccgcaccggttacaatggagagttcctgggggtcggaggaacttggaacctgaacgaggagaagaatcctgacgcggagatcgttgcctcaggagtaatcgtgggctacctcatttatacgctagtgcagatcgtcacattcttgtttggtaccacggagcacaaacgcgctttgtctgagatcgtgatgaacttcgtcggagtgttcctatggattgcggtgggcgctgtggcattacactactggggaggataccagggcgagcaccagttccagtttgtcttcgctgagaaacaggttggcctggccgtcggagctctatgcgtcataaatggagccatatatttattggacacggcactttctgtcatacatttcacaaaggaaatgtaa

SEQ ID NO: 21 - DNA, 474 bp, Phlebotomus papatasi
atggtgtctgtggaaactattggttcagtttttctcaaagtgttcaaattggttctcaacatagttatcctcattctctaccgaactggctatggaggtgatttcctgggtgttggtggaacatggaatttgaacgaagagaaaagtcctgatgcagaaattgtagcttcaggtgttattgttggtttcatgatttacacctcagttcagttaataacatatgcctttggtacgacagcacacaaaagagaactctcagatacgatcatgaacgttgtgggtacattcatgtgggtggctgttggtggaactgcccttcactattggcacggatatatgccggatcatgatttccttcatgtggccacagagaggcaagttggtcttgccatgggagccctatgcatcatttctggtgctctctacctggttgacaccgtattagcgtttgtacacttcgcgaaggatgcctaa

SEQ ID NO: 22 - DNA, 450 bp, Culex quinquefasciatus
atggcgatactaaaggcactcaacttggtgatcctgatcatctaccgcaccggttatggcggcgacttccttggcgtcggtggcacctggaacctgaacgaggaaaagagtcccgatgcggaaatcgtagcatcgggagtgttcgtcggttacttcatctacacctcggtccagctgattacgttctgcttcggaacgaccaagctgaagcgcgaactgtcggacaccatcatgaacgtggtcggaacgtttatgtggatcgccgtcggtggcacagccctgcactactggcacggattccagcccgagtatgacttccagcaaattacctcggaacggacggctggtctggctatgggatcgctctgcgttgtcaccggtgctctctacctggccgattccgtcctggcgttcattcactacgccaagcacgagaacagcaagtactaa

SEQ ID NO: 23 - DNA, 474 bp, Anopheles gambiae
atggtgtctgcggaaacgattggttccatttttataaaagtgttcaaagtggtcatcaacattgtagtactgatcatttaccgaacgggatatggaggagatttcctcggtatcggtggtacctggaaccttaacgaggagaagagcccagatgcagaaatcgtcgcgtcaggtgtatttgtcggctttatcatctataccggagtgcagctacttacttttggtttcggtaccaccaaacataagtacgagctttcggacacgatcatgaacgtggtcggtacgttcatgtgggttgctgtcggtggtactgcactacactactggcacggttatctagcggagcacgactttgaaaacatcacttccgaaagaacggctggattggcattaggtgccttgtgtgtcatcaacggagccctctatctagcagattctgtgcttgccttcattcattacactaaatatgcctaa

SEQ ID NO: 24 - DNA, 441 bp, Aedes aegypti
atgatagcactgaacctggtggttctcatcatctaccgaacgggctacgggggtgatttcctcggcgtgggtggcacctggaacctgaacgaggagaaaagtcccgacgcggaaattgtggcctccggagtgtttgtcggctacttcatctacacctcggtgcagctgattacgttctgcttcggcacgaccaagctgaagcgggagctttcggacactataatgaatgtggtcggtacgttcatgtgggtcgccgtcggaggcacggccctccactattggcacggttatctggcggaacatgacttccagcacatcacctcggagagaacggctggacttgccctgggatcgctttgcgttgtgaccggtgccctgtacctggccgattcggtgctggccttcatccactacgccaagcacgagaacagcaagtactaa

SEQ ID NO: 25 - DNA, 477 bp, Dendroctonus ponderosae
atggccttagagacgatagcctccattatagttaaactagtgaagctggtattgaactttatcatcctggttctctatcgcgtgggcttcgctggcgacttcttgggagtcggaggaacatggaacttgttcgaggagaagagctcagacgtggaaattatcgcctcaggagtttttgtggggtatttcgtctacaccgcagtgtccctgatcagtctctgccttgccagcagcgaaaataaaaacactttcacggatattttgatgaacattgtcggagtatttctttggatcgctgtgggagccacagctttgcactactggcatggttatcttagtgagcacaagtacacttatgtgaactcagaaagacaggttggtctcgctttggggtccttaagtgtgctcaatggtgcagtttacctcgttgatagcgtcatatcagtaatattcttaataaaagccaagttgcagtag

SEQ ID NO: 26 - DNA, 468 bp, Pediculus humanus corporis
atgagtagaaaagcaacaattggttcactcgtgtcaaaatgtggaaaaatcgtgttaataatgataatattaatattatacagagttggagacaatggaaaatttttaggcgtgggtggcacgtttaatttaaacgaagaaaaagcagtagatgttgaaataatggcgtctggaatattcgttggttacctagtttataacctatccgtgttaataacatatttattaactggagaaagaataatcaatgattgcataatgaacgttcttggattatttatgtggatagcagttgctggtactgcattgcattattgggacaactacgcccatcaacatcaatttgacataaccggaaatgaaaggagttcgggtttagcattgggaagtttatgcgtttttaatgctttcttacatcttgcggattctgcatttagtattaaattatatttggataaatcgaaataa

SEQ ID NO: 27 - DNA, 480 bp, Nezara viridula
atggacatccaaactattgcaacaatatttattaaagttttaaaacttgtaattaattttatcattcttatgctctaccgttatggcgggaaacaaggatttctgggtgtaggtggaacgtggaacttgtatgaagtaaaaagtgctgatgctgaaattattgcatctggagtttttgtgggatttttcatctacacatcagttatcttaatttcatactgctttggaacaacaaagcacaaatatagtgcagtagatataataatgaatgtgagtggaactatactgttcaccgctgtaggaggcatagcattacattactgggttggtttccaggatgaaaaccattatgtgggaatttctcaagaaaaacaaataggacttgcattaggcagtcttagtgtagtatgtgctgctatctaccttctcgatacagttttatcatgtatacatattgcacacaagcatgctattgcttaa

SEQ ID NO: 28 - DNA, 483 bp, Lygus hesperus
atggagttggaaaccatcatcacaattttcatcaaagtcctcaaattggtgctggacttcataataatgatgatgtaccgctacggtggaaacagcggcttcttaggtgttggaggaacgtggaatctaaacgaagtgaaaaacccagacgtcgaaatagttgcctccggagtttttgtgggctacttcatctacacagcagtcatcctaatagcattttgcttcggaacgactaaacacaaagcgagcgtcgtggacttcataatgaacataacaggaatcatcatgttcattggcgtcggaggtatagctctgcactattggtgcggataccagaacgccaaccacgcacaatccatatccacgacaaaacaagtaggaatcgcgcttggaagcttatgtgtggtggaaggagcagtttttctgttggataccgtcttggcatttttacacctctacaagaaacgcagcagcgaatggtaa

SEQ ID NO: 29 - DNA, 480 bp, Euschistus servus
atggatatccaaactattgcaacaatatttattaaagtactaaaacttgtaattaacttcatcattttgatgctttaccgctatggtggaaagcaaggatttctgggggtaggtggaacatggaacctgtatgaagtaaagagtgcagacgctgaaattattgcatcgggtgtttttgtaggattttttatatatacatctgtcattttgatttcctactgcttcggaacaacaaaacacaaatacagtgccgtggacataataatgaatgtgtgtggaactatactgtttactgcagtaggaggaatagcattacactattgggtcggcttccaggatgaaaaccattacgtgggaatctctcaagaaaaacaaattgggcttgcactgggcagcctttgcgtcgtctgtgcagctgtctatctcctagatacagttttatcttgtattcatatcgcccacaagcatgctattgcttaa

SEQ ID NO: 30 - DNA, 405 bp, Halyomorpha halys
tatcggtatggtggaaaacaaggatttctaggagttggtggaacatggaatctgtatgaagtaaaaagtgcagatgctgaaatcattgcatcgggagtttttgtgggattttttatatatacctctgtcattctaatttcgtactgtttcggaacaacaaaacacaaatatagtgctgtggatatcataatgaacgtaagcggaactatattgttcactgcagtaggaggaatagcattacactattgggtcggcttccaggatgaaaaccattacgtcggcatctctcaagaaaaacaaataggacttgcactaggcagtcttagcgtcgtttgtgcagctatttaccttctcgatacagttttatcatgcatacatattgcacacaagcatgcaatcgcttaa

SEQ ID NO: 31 - DNA, 480 bp, Megacopta cribraria
atggatatccaaactatagcaacaatttttattaaggtcttaaaacttgtaattaattttattattttgatgctctatcgatatggtggaaaacaaggatttttaggaataggaggaacatggaacttgtatgaggtgaagagtgcagatgctgaaataatagcatcaggggtctttgttgggttttttatatacacatcagtactgctaatatcctactgctttggaacaacaaaacataaatacagtgcagtagatatgataatgaatgtaagcggaactatattatttaccgctgtaggtggaattgcattacactactgggttggatatcaagatgaaaatcattatgtgggtatttctcaggaaaaacaagtaggattggccctaggaagtctgacaattgtttgtgcagctatttatcttctggacactgtcctatcttgtatacatattgcacacaaacatgctattgcctaa

SEQ ID NO: 32 - DNA, 474 bp, Manduca sexta
atggtgtccgttcagacgatagcgacaattgtcgttaaagctttcaaaattgtattgaacataattatcctggtgctctaccgaactggctacaacggcgagtttctcggagtgggcggtacatggaacttgaacgaagaaaagaacccagacgctgagatcgtcgcgtcaggagttatcgtcggctacctgatctacacgctggtgcaagttgttaccttcctgtttggcaccacggagcataaacgcgccatgtctgagatcgtgatgaactttgtgggcgtgttcctgtggatcgcggtgggcgcggtggcgctgcactactggggcgggtaccagggcgagcaccagttccaattcgttttcgctgagaaacaagtgggtctcgcagtgggtgctctctgtgtgatccaaggcgcagtgtacctcttagatacagcactatccgtcatacacttcactaaggagatgtaa

SEQ ID NO: 33 - DNA, 480 bp, Euschistus heros
atggatatccaaaccattgcaacgatatttattaaagttttaaaacttgtaatcaattttatcattttgatgctctaccgctatggtggaaaacaaggatttctcggagttggtggaacatggaacttgtatgaagttaaaagtgctgatgctgaaattattgcatctggagtttttgtgggattttttatatatacatctgtaatccttatttcatactgctttggaacaaccaaacacaaatatagtgcagtagatattataatgaacgtgagcggaaccatcttgttcaccgcagttggagggatagctttacactattgggttggcttccaggatgaaaatcattacgtgggaatctctcaagaaaagcaaataggacttgcgttgggcagtcttagcgtagtttgcgcagctatttaccttcttgatacagttttatcttgtatacatatcgcgcataagcacgcgatcgcttaa

SEQ ID NO: 34 - DNA, 480 bp, Piezodorus guildinii
atggatatccaaactattgcaacaatatttattaaagttttaaaacttgtaattaatttcatcattttgatgctttaccgctatggtggaaagcaaggatttttgggggtaggtggaacatggaacctgtatgaagtaaagagtgcagatgctgaaattattgcatcgggtgtttttgttggattttttatatatacatctgtcattttgatttcctactgcttcggaacaacaaaacacaaatatagtgcagtggacataataatgaatgtgtgtggaactatactgtttactgcagtaggaggaatagcattacactattgggtcggcttccaggatgaaaaccattatgtgggaatctctcaagaaaaacaaatcggccttgcactgggcagcctttgcgtcgtctgtgcagctatttatcttctagatacagttttatcttgtattcatatcgcccacaagcatgccattgcttaa

SEQ ID NO: 35 - DNA, 90 bp, Tribolium castaneum
atgacgagcattgaaactgtgggggccctaatcctcaaaatcctcaagttggtcctcaacttgatcatcattatcttgtaccgcaccggc

SEQ ID NO: 36 - DNA, 1147 bp, Spodoptera frugiperda
cagacgtgtgctcttccgatcgcagatactgagtgcaagtgacaatggtgtcggtgcaaactatcgcgacgattgtcgtgaagacattcaaaattgtattgaacatagtaattctggttctttaccgaactggttacaatggagagttcttgggagtgggaggtacgtggaacctcaatgaagagaagaatccggacgctgagatcgttgcctctggagtcatcgtgggctacctcatctacacactcgtgcaaatgatcacattcctctttggtaccactgagcataagagagccatgtcagagatagtgatgaacttcgtgggcgtgttcatgtggatcgccgtgggtgccgtagccctgcactactggggcggctaccagggagagcaccaataccagttcgtctttgctgagaaacaggtgggtattgctgtcggcgccctctgcgtgatcaacggcgcagtgtacctcctagacacggcactctccgtcatacatttcaccaaggagatgtaaacttagcccgtactataaataaacttaattttatttcctaactttaagtattattaagtgttttacgaacgaccctctattacccgagtatttacagtaaatagttgcaatccttttatattgtgatctgaggattttggtataataaggattatgacaaacgaataacgattatgtaagtattatattcctacacgaattaactgctgcactcagagacatttaatttttaggtacttcaactatttctttgtaacgattttgtttcaaaaacaattactaagcactgggttaaatacctaatcattaaatgactctgagtacggcggtaaatgcttattggcagtgtgtttgggaatcccgattaacagttttcttatgaacgcacgtttacgcgcgtcgacattcgaattgcagcgcgttaacgcacgttttcaaagtaaactgtgtactataatgaagaagccattttgaaataattctgtatcaatattatgctctcgtgcaaacattttctatcatatttcccaccttctgtataagttagttaatgtagaccctgttttatatgtcagttatgattttttaaaatctaattattattttgaaataattctgtatcaatatta

SEQ ID NO: 37 - DNA, 1482 bp, Ostrinia nubilalis
agttacgaggtctgacgctactcgacgatatgtatttacagggtgattcaaacgtgccactgtatatccagcttagaacctaatacgatttgaaaacgaaggacaatataagaatcaaatgcgtttattttcagaaacgatttgatatcttaggacgcggatgacaggtatacaataaagaacagcttaaaacaaaatggaactaacgaaaaaaggaaaatgaatcttaatcaaataaacattggcttcaatatgaatgacggaatgaaaataacttacgcggcacctgccctactaattcgctgaaaagataagataagtccgcatggactatgctgataacccatactatacgataagtgaaatcacccagtggcgaaacgactgttaacaaaaacagacaaagacaaagaaatctatgtgataaaaaaaagaaaaagaagaacagtgaaatctgtattttaaatcaagtgatagttccaaaataagtgaaaatggtgtccgtacaaacgattgcgacgatagtggtgaaaaccttcaaagttgtcctaaacatcataatcctggtgctgtaccgcacggggtataatggggagttcctcggagtgggaggaacgtggaacctcaacgaggagaagaatccggacgctgagatagttgcttctggagtcattgtgggatacctcatctacacattggtgcaaatcgtcacatacctcttcggcactacagagcataaaagagccctgtcagaggtggtgatgaacttcgttggtgtgttcatgtggctggctgtgggcgcggtggcgctgcactactggggaggataccagggagaacaccagttccagtttgtctttgcagagaaacaggtgggcattgccgtcggagcgctctgcgtcatccagggagcggtgtacctgctcgacacggccctctccgtcatacattactcaaaggagatgtaaacacacaccttttagcttgagtagccaaagccaactgttttttaggaaattatagaaagtccactgaagaagccaacttctacgtcttcacagcttcgagtgttttaagtcaatttgacgtacaaaatagtttatttgaatatcgaagcggattcgaaaacaaatgacgaacgatacatattttgcgtcacgtaaattttattgtatctacataattattaattacgcacgtggtgcttggtaatttcttaaaactataaacattaataataaatattttagtaaattattatagttatccttattgagagtgttattggcttttactatgttggttgttaactcaaagctttatttaaatttagattaagtattttcgaaaacattcacccttgacttatttacagtttcccaccttctgatagttaagttcataagttaacttttgtatgtgccagaattatttttaatgtatgacgttattttgaaataaatttaataacaactaaaacta

SEQ ID NO: 38 - DNA, 1035 bp, Helicoverpa zea
acacagtgacgggcgtcggtttagcgaaaacggacaaaaagacaaaagaggattcatttgttagtagtgatatcaaagtgtgagtgaaaatggtgtccgtacaaaccatagcgacgatagtcgtgaaaacattcaaagttgtactgaacatagtgatcctagttctctaccgaacgggttacaatggcgagttcctcggagtgggcggcacctggaacctcaatgaggagaagaaccctgacgctgagattgtagcctctggagtcattgtcggctacctcatatacacgctcgtacaaatcgtcacgttcctgtttggcactactgagcataaaagagcgctgtcagagatagtgatgaacttcgtgggcgtgttcctgtggatcgcggtgggtgctgtcgcgctgcactactggggcggctaccagggagaacaccagtaccagttcgtgttcgctgagaaacaggttggtttggcagtgggcgccctctgcgtgatcaacggagcagtgtacttgctagacacggcgctatccgtcattcatttcaccaaggagatgtaacctagtgcttaatgttaataaacttaattttatttaattctaagtacagctaagttgacctaagtgttttacacgaaagaccggtaatttacgaataaaaatagttttttgtttcattcctgtttcgaatgagatctgaggattgaacataaaacgatttatgattaaaaataataaactttaacatatattttgaacaaccgtcacttttatcaatattactttattcttaacttaaggaaaccgaaacgataatacaattcagttaaataactatcaaagcataagttttgttacaaaaatttgaaaaaaaaaaaaattgtttatggtgttcataatgctctcgcgcaaaacattgtctatttcccaccttcttcctaagttagtattgtagaacatgttttatatgtcagttataattttgtaacgtaaatattattttgaaataaattaaagtgtaataaaaaaaaaa

SEQ ID NO: 39 - DNA, 1564 bp, Phyllotreta cruciferae
gttttctaacagcacagtcctatcaaataaaaatatttagacaatgatataccattgaaatacatacaaactttaacctgttactcattctttttttatagtcgatttttgttaaattcattttaaatctacaagattggtatcccttatgataacgcccatgattctgtagaattcatgatataaaaggtgataaatattatttgctgagtttgtcatgaaatttattactttcagtgttagtatttactgtacctatacctacaatttttatgagctaaatcttatctatcgagataaatatcattatctatattgtctcacctcattttcatttgatacgagcaaaaatacgttattagttattattcttctagagaaaataactgtagaaaagtgtgaactttcgtaaaaatgacaagtatagagaccgtgggtaccatcgtacttaaattattgaagttggtaataaacttgatatgcataataatatacaggaccggttaccagggctacttcttgggagtaggtggaacttggaacttaaacgaagaaaaaaatcctgatgcggaaattgtggcatcaggaatttttgtcggctacatgatctacactaccgtatctcttataagtttctgtttcgccagcggtgatcacaaaacatctttcaccgatattttaatgaacatagtcggtgtgttcatgtgggtggccgttggagcaacagcacttcactactggcttggttatttgtcagaatataaatacacgaccgttgattccgagcgacaggtgggattggcgttaggagcgttatgcgttataaacggggcagtctatctagtggacacggtgttgtctgtagtatttttgataaaagctaagatgcagtaataatttttaatgaaatatttattaattttttacctatctattttatagaagatttttttagtgcaaaatttcctcgtaattcatgacaatatttcatcattgcagtaatctttttttatacattttgatatgtcaacatttttagtactttaatgcactctaggtcttatagtcttgcattcgtgtggataaatgtttattgtaatgtatttggttcaccctatatttaatgaaatttgcagtatattccagtaagaacaaaaggtaatgaatattttgaaatattgtctagtaaagactattttttccaaacttaaattcgattttaacaaaaatcccttaaattcgctgatgtttacctaaaggaaatctcaacaaaaacttgacaaacaatttgaaatacatatttttgtagtacattatcatagtatattatgttaaatttatttagccgttctaaagaaaaaaaaatggctaggtaataatagaacttcatagttttaggatgccgacattgtaattaattcgcatcttataatattgtataaatacaagtaatcatgtttacaataaaatttaaaatgtgtttatttttttgaattgagcggggtgactaaaatttatggtgttacagggtgctttttgaatgtttcttatgtttcgacagcgt

SEQ ID NO: 40 - DNA, 1212 bp, Phyllotreta striolata
atcattatctatattgtctcacctcatttatcatttgatacaagcaaaaatacgttattagttattattctaatagagaattcttctagagaaacatcagctcaaaagtgtgaccatacgtaaaaatgacaagtatagagacagtgggtaccatcgtacttaaaatattgaagttggtaataaacttgatatgcataatactatacaggaccggttaccagggctacttcttaggagtaggaggaacatggaacttaaacgaagaaaaaaatcctgattcggaaattgtggcatctggaatctttgtcggctacatgatctacactatcgtatctcttataagcctgtgtttcgccagcggtgatcacaaaacatctttcaccgatattttaatgaatatagtcggtgtattcatgtgggtggccgttggagcaacagcacttcactactggctcggttatttgtcagaatataaatactcgagcgttgattccgagcgacaggtgggcttggcgttaggagccttgtgcgttataaacggtgcagtctatctagtggacagtgtgttgtctgtcgtatttataattaaatcgaagatgcaataataatttttaatgaattatttataaatttttttatctttctatattttttaaaagatttctgcaaactgcaaactgcaaactttcctcgtaattcatgacaatatttcatcattgcagtcatctttcatgtacatttttaatacatattttaatgcactcttaagtcttatagtcttatggcattcgtgtggataatgtaatatggatgtttacttcattcaattcttttctgttcagtttattaagtgcaataattgataataaaaaggtaacgaatattttgaaatattgtctgcggtgagtcttatggcattcgtgtggataatgtaatatggatgtttacttcattctatatttaataaaatgttcagtttatttagtgtaataattgataataaaaaggtaacgaatattttgaaatattgtctgcggtttaatgactaatgaatttgaatttggcgaaaatataataaattcgttactgttgtcctaatgtaaatctcaacaaaaattggcatgcaatttaaaatttgaagtttttgcactacaatatcatattttatgttaacttttagttaaacgttattttaaagaaaaaatagttcca

SEQ ID NO: 41 - DNA, 1375 bp, Acyrthosiphon pisum
gagtatagtgcgttcacacgaaaccaccgacgataggtatgggtaggtcgtaaaacacaccgtcataaagataacgcgcgaacgtggagaggcgtaaagctctcatccaccgcgttcaacgcatcgcgcaatcgactgcagtggcagcgaactgccgtcagtggcaatatacctagatactttgtgtatgcgtgcgcgatttgtcacacaacgattacattgttattttatatatagacatataatcactggagcgaattcgcgatggtcaacatagcaagcgtgggcacggtggtcatcaaagtcgtgaaattggtgttgaacataatcattttggtgctgtaccggaccgggtatcgcggtggatttttgggagtcggcggaacgtggaacctgaacgaagagaaaaacccggatgcggaaatagtcgcttccggcgtgttcgtcggtttcttcatctacaccgtcgtgattctaatatcgtacgggttcggatccaaccaccagaagaaaaccctagtggatataataatgaatttcgtcggcatgttcatgtttatcgccgtcggcggtatagcgctccactattggattggttatcagaacgaaaacaaatacatcagcgtaacttccgaacgtgccataggcatcacggtgggcgtgctgtgcgttatttccggtgcaatttacctggtggacaccgtactgtcgtttatacattttgccagagaaatggagttcgattaaaaggagcgactatggcaaatttaagttaatttaaatatatagctatactttacacaaaagtttgacggttttgctcttcgagtgcacaattaaacacacactattttatatttctattatgtatgtatacactgaaaatctgtctgtacaaatataatatattgtatacaatccgtaaggatatatgaaccgtgaaatgttcacacgagcggatagtctacatgcatgcatgtgtgtgtgaatacaacagtgttataaaaccattcaaacatataaataatataatatgtgctttgtgaaataatatcaaataattaataattaactttttttctactcgttatatacctgctactatacttacttatttaaacctaacggcattccatccatatataagttataatagtgttttgtaaatataaatattcttataatacctatgtagttatatatttatataatattatgttatattaatagttagttagttatattataattatatttaaataccgtgtataagtataaatacattaccccaacgatcgtttgtatttaaatacagaattaatgtgaatatttttttaatacatatataatatcatatataaaaaaaaaaaaaaaa

SEQ ID NO: 42 - DNA, 645 bp, Apis florea
tacttcagatccacctgttactgttcgtgaagagtgagcatcgaaacgtttaaacatggtgtctctggaaactgtcgcgggtatagtgataaaagttctcaagttgattattaatctaataatcctgatcctgtatcgaacgggatacaagggagaattcctgggtgtcggagggacatggaacttgaacgaagacaaaaatccagacgcagaaatcatcgcatccggagttttggtcggatttttcatatacaccagcgtcgttttaatcacttactgctttgggagcatgtatcacaagaagacacttgtcgaaattataatgaactttgttggaaccttcatgttcatcgctgtggggggtacagcgcttcattattggcatggttatcaaccggaaaataaatacgattcgattgttcaagaacgacagatcggcttagctgttggagcgttatgcgttatcgagggcgcaatgtatctcgtcgacttaatattgagttttctccattttgcgaaaaataccgacaatttttagaattggaataaaattaagaaatttcatataaaaaaaaaaagcagtatagaatcaacgtatttttttaaattggtatcatcatcgataataatcctttcattaaaaat

SEQ ID NO: 43 - DNA, 683 bp, Apis mellifera
cttagtactctcgaaaccagcacgtttttacttcagatccacctgtttctgatcgtgaagagtgagcatcgaaaagctaaacatggtgtctttggaaactgtcgcgggtatagtgataaaagttctcaagttgattattaatctgataatcctgatcctgtatcgaacgggatacaaaggagaattcctgggtgtcggagggacatggaacttaaacgaagataaaaatccagacgctgagatggttgcgtccggagttttggtcggatttttcatatacaccagcgtcgttctgatctcttactgcttcgggagcatgtatcacaagaagacacttgtcgaaattataatgaacttcgttggaacgtttatgttcatcgctgtggggggtacagcgcttcattattggcatggttatcaaccggaaaataaattcgacacgattgttcaagaacgacagatcggcttagctgttggagcgttatgcgttatcgagggtgcagcgtatctcgtcgatttaatattgagttttctccatttttcgaaaaataccgacaattttttagaataaagtcgaaaaatttcgtataaaagaaaagcagtatagaaccagtattttttaaattgatatcatcatcgataataatcctttcattataaataaatattcaatatacatacat

SEQ ID NO: 44 - DNA, 2177 bp, Bombus impatiens
catttccgcgatttgctccggggtgataactaagcccagcatctaaaataaatattggacattagaaatatttaataatattattcgtaaaagactcaacgaaagactatataaatcagaactgaaaatttgtaaatttcaagcttgatacgatggcgaatgctaattaacgtctagtcactaatttctttttagaaagtacattcgcataatgatttacatattttacgatatatctttacactgtttcttcagataaaattgaatttttaaaaccaagttctattcaagtcaattttgtaattatgtgcagtggaggacttttaagataattaggtcaatggcatttaaaaaaaatggtcagggataagaatacaataacgtttcatgataattttattgaattaatcgtatgaaaacaagcattaagaaaataaagtaacaataaacgttacatttcgaggtatattatcgttcgttttctactggtcttatccctctgtcgacgtacgctgtatcatctgattagataaagagggcttggtaccgcgtcgaaaacaaccagcacgcttttatttcaattagttcacctatcccaagcgtgaagagttcatatagaaaaagtaatcatggtgtctttagaaactattgcgggtatagcgataaaagtgcttaaactggttattaatcttataatcctgatcctgtatcgaacaggataccaaggggaattcctgggtgttggaggaacatggaacttgaacgaagacaaaaatccggacgcagaaatcgttgcgtctggagttttagtgggatttttcatatacaccagcgttgttctaattacttattgctttggcaattcgtctcacaaaaagacacttgttgaaattataatgaacttcgttggaaccttcatgttcatcgctgtaggtggcacagcactccattattggcacggatatcaatcagaaaataaattcgatacgattgtccaggaacgacaggtcggcctagctgttggatcgttgtgcgttatcgagggtgcagcgtatctcgtcgatctaatattaagcgttctacattttgcaaaaagccacgatgaatatttagaataaagtcacgtttcacgtaacatatagaatagtaatctcgccacatttttttccaaacgatgtcataacagaaagataaacaaatcctccgacgtttgaaataaaagttcagtatatgtctgtcatctgcataatgccaaaataatttatctaagaaaggaagaggattaaagttaaagtaaatcgttgttaaattaaaaattagattacattttttataattacaattataattacaactttgtacttgaattatcgacaagatgtactactgcatatacaatctatatgatatctgtctatattatagatagtaaaatgtagaaaacagtctttaatttttatataatgttgctacaatcttgtcagtaatttcgtttcatcatttatacgacaatttaatatatctattttaacgaattttccaaatcagttgtttcaaatcttttctcgtaaatatacgtatattctttacaaatttaagaaaagaattctatccaacgaaagagattaaaagaacaactttcgaatgttctacaatatacgtccacggtttgataatatgaataaagtaacttatccggagaaaaattattcgcagacaattcagtacatatctcgatattaagatcgattactttcatcgaattttaaagcgtccaatgattagtttactctatgatttaataaacatgcctcttactcaagtacctagacttttcaaagacttcagttttcttttgaaagtacacgaaatggtaatactactttttatcgaagaaattgcgaatacaaatcgaaatcgaaactacattttcttggccgtttagatcatagctggtgtttgcatccacgagctgttaaaccgcaaggaattcacgacaccaagatggttatgggtcatttactgcatcgacgcgtccacgtactttatcatctatttttccatcgcaatgactcacgtttttgaaaaacctcgcagattaccagttagttgcacatgagctttcttaacaacgtaaactttcctaataacgtaattgaattacggaaatttgagga

SEQ ID NO: 45 - DNA, 2156 bp, Bombus terrestris
catttccgcgatttgctccggggtgatagctaggcccagctcctaaaataaatattggacattagaaatatttaataatattattggtaaaagactcaacgaaagactatataaatcagaactgaaaatttgtaaatttgaagcttgatgcgatggcgaatgttaattaacgtctagtcactaatttctttttagaaagtacattcgcataatgatctacatattttacgatatatcttcacactgtttcttcagataaaattgaatttttaaaactaagttctattcaagtcaattctgtaattatgtgcagtggaggacttttaagatagttaggtcaatggcatttaaaaaaaatggtcagagataagaatataataacgtttcatgataacttttttgaattaatcgtatgaaagcaagcattaggaaaataaaataacaatgaacgttatatttcgacgtatattatcgttcgttttctactggccttatccctctgtcgacgtacgctgtatcatctgattagataaagaaggcttggtcccgcgtcgaaaacaaccagcacgcttttatttcaactagttcacctgtcccaagcgtgaagagttcatatagaaaaagtaatcatggtgtctttagaaactattgcgggtatagcgataaaagtgcttaaactggttattaatctcataatcctgatcctgtatcgaacaggataccaaggggaattcctgggtgttggaggaacatggaacttgaacgaagacaaaaatccggacgcagaaatcgttgcgtctggagttttagtgggatttttcatatacaccagcgttgttctaattacttattgctttggcaactcgtcgcacaaaaagacacttgttgaaattataatgaacttcgttggaaccttcatgttcattgctgtaggtggcacagcactccattattggcacggatatcaatcagaaaataaattcgatacgattgtccaggaacgacaggtcggcctagctgttggatcgttgtgcgttatcgagggtgcagcgtatctcgtcgatctaatattaagcgttctacattttgcgaaaagccacgatgaatatttagaataaagtcacgtttcacgtaacatatagaatagtaatctcgccacatctttttccaaacgatatcataacagaaagataatcaaatcctccgacgtttgaaataaaagttcaaaatgtgtctgtcatctgcacaatatcaaaataatttatctaagaaaggaagaggattaaagttaaagtaaatcgttgttaaattaaaaattagattacattttttataattacaattttgtatttgaattatcgataagatgtactactacatatacaatctatattatatctgctatattatagatagtaaaatgtagaaaacagtccttaatttttatataatgttgctacaatcttgtcagtaatttcgttttatcatttatacgacaatttaatatacctattttaacgaattttccaaatcggttgtttcaaatcttttctcgtaaatgtacgtatattccttacaaatttaaggaaagaattctattcaacgaaagagaaaagaacaactttgaaagttccacaatatacgcccacggtttgataatatgaataaggtaacttatccggagaaaaattattcgcagacaattcagtacgtatctcgatattaagatcgattactttcatcgaattttaaagcgcccaatgattagtttattgctttaataaacatgcctcttactcaagtacgtagacttttcaaagacttcagttttcttttgaaagtacacgaaatggtaatattacttttcatcgaagaaattgcgaatacaaatcgaaatcgaaacaacattttcttggccgtttagatcatagctggtgtttgcatccacgaactgttaaagcgcaaggaattcacgacaccaagatggttatgggtcatttattgcatcgacgcgtccacctactttatcatctatttttccatcgcgatgactcacgtttttgaaaaacctcgcagattgccagttagttgcacatgacctttcttaacaatgtaaactttcttaataacgtaattgaattacggaaatttgagga

SEQ ID NO: 46 - DNA, 1430 bp, Nasonia vitripennis
ctcctcgacgccacttgcgcgttagtccgtgtcgctcgttgcttcgattttcacctcatgtcgcgctgctcgcgattttaactctggtttttgcttggacgagtgtgcgtgtgtgaaaatctacagctgaagatggtgtccgtacagacgatcggcggcatcgtcgtcaaggggcttaaattgctactgaatttgatcatcctcattctctaccgaacgggctacagcggagaatttttgggcgtgggtggaacatggaacctgaacgaagaaaagaatccagacgcggaaatcgtcgcttccggtgttttcgtcggctttttcatctacaccagcgtcgtgcttatcagcttttgctttggcaccatggatcacaagaagtcgatcgttgaagtcatcatgaactttgtgggcatgttcatgttcctcgcagtcggcggcactgccctgcactactggcacggctacatgtcggagcacaagtacctgcacgttgcaacggagagacaggtcggcctggcgctgggatctctgtgcgtgctagaaggcgcgctgtacctcgtcgatctcatattgacgggtttgcacatcgccaaggaggagttcgcctaacttcacgagaacttggaagaaacacttcaatttttttacagaaaactagatgtatcaatttatcaccgatttttttttttttttaataaaactgcatttatgtttctcgaggggtgggtgaacctttagagaagtttgaatgaaatagttaaatgtatgatattttattcgacgtttgcgtagagacacgtgctcgcattcttttgtaaaccaatttttttttcttgacagtacaatgttcttacgttcatttcgtttatcgctcacttatcattttgtatttttaatcgacttcttaatcgaaatcaataagtaagtagggttttcatcgcttgttctcatgtaagttgtagggaactaggataagcatatttcgcgtaggcttaagctactaatactttgtttgtaacacttttatattgcgaaagcatctgtattcaatctttttacttcgttgtataatgaaagggtaaataataaatgacaaataaaagaattctcctctcgcatcttgttctaaatcaccgataaaaactgaagttcgaaagttctgtaactctttataacctcctcttgcgccaacttcaagttactttgagcagtacttccagcgactttatcgactttcagcggactaatcaatcaataaaatatcgcaatggttaaatcctgcaacgaactctggtgcgatccccacttcagtctcaaattcgccgtattggtgagtaatcgataaattgatattgctataatggaaaatgttaacctaataaacaacacttatgactgaaccaaaatattgtccctgtgcagatctt

SEQ ID NO: 47 - DNA, 738 bp, Megachile rotundata
gttcacctgttttacatcgtcgagtattcgcgtgcaagaaaatcatggtgtctctggagactatcgcgagtatagtgatcaaagttctgaagctggtaattaatctcataatcctgatcctgtaccgaacaggatacaaggatggagagtttctgggtgtcggtggcacgtggaatttgaacgaagataaaaatccagatgcagaaatcgtggcttctggtgttctagtgggcttcttcatatacaccagtgttgtactcatcacttactgttttgggagcaccaatcacaaaaagacccttgttgaaatcctgatgaattttgttgggaccttcatgttcatcgcagtaggaggaacagccctccattactggaacggttatcaaccagaaaacaagtacgacacggttgtccaagaacgacaggttggcttagctgttggatccctctgcgtcatcgaaggctccgcgtacctcgttgatttaatacttagttttctacattttgcgaaaaatcgagatgaaatgttcgactaaatttgatgtatataaaattgttcattgtatcatcatagagcaatcttcacgaagagcaagaataatcagaacctttcttagagtggaattagaacattcatacaacatgttttttaggtattaagtttttggaattagttttaggttaagtttaggaagacaggttcattttgaaattaaaaagagaggttcgtttttaatg

SEQ ID NO: 48 - DNA, 1052 bp, Spodoptera exigua
aagataaccagagtcatacagtgacgggctacgttttggcgaaaacggacacaaagacaaaagaagtgtaaatataaattataattgtggtgaatactgagtgcaagtgacaatggtgtcggtgcaaactatcgcgacgattgtcgtgaagacattcaagattgtgttgaacatagtaattctggttctttaccgaactggttacaatggggagttcttgggagtgggaggtacgtggaacttgaacgaggagaagaatccagatgctgagatcgtggcttctggagtcatcgtgggctacctcatatacacacttgtacaaatgatcacattcctgtttggtaccactgagcataaaagggcaatgtcagagatagtgatgaacttcgtgggtgtgttcatgtggatcgccgtgggtgccgtagctctgcactactggggaggttaccagggagagcaccaataccagttcgtgtttgctgagaaacaggtgggtattgctgtcggtgccctctgcgtgatcaacggtgcagtgtatctcttagacacggcactatccgtcatacacttcacaaaggagatgtaaacttagcactaatattataataacacttaattttattttttaattttaagtatacctaattaagtgttttacgaacgattcgaaccattatttttattcgagtaattgcagttaacagtttaaaaatccttttataattcgatctgaggatttaatataataagggttatgacaatgaatatctataatataaactttccttcacgaatatttcttgtttgtcgaagtgtgcgtttaggaatcccgtttatgacagttctatataagcacccggtaacgcgcgttgacgctcgaattcgcgcgtaaacgaacgtttttattttgggttggaaaagccatttcttcattttcgtaataaatctgtaacaatacatattatgctctcgtgcaaaacatttttctataatatttcccaccttctgtgtaagttagttaatgtagaccctgttttatatgtatatgtcag

SEQ ID NO: 49 - DNA, 1079 bp, Chrysopa pallens
cgatatataaaggaaatatttgcgtattatttgatgttttgaaacgacgtatcgaaattcttatctctcgaaaaagctaatatgataagcttttcgatatgccctctattttcatgtcaagcatgagcattccaaatttcataacatttgagttatttattcgcgaaaattgtgtgttatcattatgtacattttaattttagatcgttttttattccataaatttaaataaaatatttctttacagtattaaattttattactttttattaaaatagattaaacctgcctgcgtgcgttaaacgccttgcacacttgctaacggcggtcctcgttaatgattttagttaactttcaaatggccctaaagatcaatattaaagttaaaaaaagttatcatttttagcaatcagcaacggtagtttgagatattttttttcgataaaaagcttatttttgcgagcgatttaaatatttgtcaagttaaccactgattaagaatgatctataaattttcacgaatataatgtaaaacggctaacacagtatccaatagatacgttgcacctgaaattacacataaaataccaactgccagaccttttgcacgttcaccaccatctaattgaccgtgattttcattttgataaccagaccaataatgaattgctactccaccaacagctaaccacatgaatacaccaacaaaattcataattatatcaactggctctcgtttatattctttggatccaaaacagaatgcaattaatgttactgatgtatatatgaagtatccaacaaatacaccacttgctactatttccacatctggatttttatcctcatttaggttccatgttccacccacacccaaaagttgtccatcgtatccggttcggtataagattaagataattaagtttaaaagcaatttaaaactttttattacaacactaccaacagtttgtatcgacaccatctttaaaacacaggttcaaactttttttaaaattgtatattctaattttaataattttacttaatatttcaaaaaaaaatactcatacaaaaattct

SEQ ID NO: 50 - DNA, 1380 bp, Bombyx mori
taaatgaattgacagttggcgtataagttgtcgtcggcggacacaggaacagtttttaatagtgataacttgtgatattttagtgtattaatatctcatttaaaatggtttccgttcagactatagcgactatagtcgtgaagacattcaaaattgtcttgaacattataatcttagtgctctaccgcaccggttacaatggagagttcctgggggtcggaggaacttggaacctgaacgaggagaagaatcctgacgcggagatcgttgcctcaggagtaatcgtgggctacctcatttatacgctagtgcagatcgtcacattcttgtttggtaccacggagcacaaacgcgctttgtctgagatcgtgatgaacttcgtcggagtgttcctatggattgcggtgggcgctgtggcattacactactggggaggataccagggcgagcaccagttccagtttgtcttcgctgagaaacaggttggcctggccgtcggagctctatgcgtcataaatggagccatatatttattggacacggcactttctgtcatacatttcacaaaggaaatgtaattttgttatatttttttttaataaataatatagcgaaaatgccattatttacttaagttacgattaatgtttctttatcgactaaaatagttttaacgaacgaattaagtagaatttaatgacgacaaaggacgtagaagctgttagaagaagttcccaagatttttattcggaaaaatgtacgatatcgctcgtagattagctttgactttaggagtttcgaatagggataaaaatcgggaatattgtctgttcgtcatcacatcctcaggacttcgagcgaaaccactgtaacgacatacatgaaaagaatgtatgtataatatgttaattaatttgcaaaacgttttgtaaactttacaattatcgacttttcctgatcacgcagaaagttacctggacattaaaaaaatctaattgtcggtaattgtaaaatgcgggtaataaacgcaagattgaaattttaaagtattttgaaaatatacattctttttactgtttactattttcgaaaatgacatattattatacattgtagattacgtgtaagtaagtaagttgatctatagatcatttccgtttgccattattattcttaccatagacaaagcatcatttttttatgctctcatagaaaacattgactttttctgtatcttcccaccttttgtaatgtattcactgagtttatttatgtttttaacgaaaccagtttttttttgtaatgtaaaattttattaaataataaatgttgtaataagaaaaaaaaaaaaaaaaaaaaaaaaaaaaaaaaaaaaaaaaa

SEQ ID NO: 51 - DNA, 700 bp, Phlebotomus papatasi
ttagagataaacacgcatcttagcacttgttccgtagcttttgtcaaagccagttactgtgtatctcaataggaaagtggaagattttttttcaaatttgtgaaaaaaagaacttgcaagaaaactatagcttttcacgcataaggaaaagggattattggtgaaaatggtgtctgtggaaactattggttcagtttttctcaaagtgttcaaattggttctcaacatagttatcctcattctctaccgaactggctatggaggtgatttcctgggtgttggtggaacatggaatttgaacgaagagaaaagtcctgatgcagaaattgtagcttcaggtgttattgttggtttcatgatttacacctcagttcagttaataacatatgcctttggtacgacagcacacaaaagagaactctcagatacgatcatgaacgttgtgggtacattcatgtgggtggctgttggtggaactgcccttcactattggcacggatatatgccggatcatgatttccttcatgtggccacagagaggcaagttggtcttgccatgggagccctatgcatcatttctggtgctctctacctggttgacaccgtattagcgtttgtacacttcgcgaaggatgcctaatcttcacctcacattttgcaatctgcaccaaaatcatcaatccaaatcattcaatcaaaa

SEQ ID NO: 52 - DNA, 450 bp, Culex quinquefasciatus; identical to SEQ ID NO: 22

SEQ ID NO: 53 - DNA, 951 bp, Anopheles gambiae
atgagtatcgttccctgggcagagtcaattcaaccgtttcattcgtcagaagatcccagtcctcgcataagcagtgaaaagattaaacagccaaaccgctaatttgaggttacgctacaggtgtcgcaacaacaagtggtcatcgtggtccttcgctgctatacgcaactgtcccacgcatcaaaatggtgtctgcggaaacgattggttccatttttataaaagtgttcaaagtggtcatcaacattgtagtactgatcatttaccgaacgggatatggaggagatttcctcggtatcggtggtacctggaaccttaacgaggagaagagcccagatgcagaaatcgtcgcgtcaggtgtatttgtcggctttatcatctataccggagtgcagctacttacttttggtttcggtaccaccaaacataagtacgagctttcggacacgatcatgaacgtggtcggtacgttcatgtgggttgctgtcggtggtactgcactacactactggcacggttatctagcggagcacgactttgaaaacatcacttccgaaagaacggctggattggcattaggtgccttgtgtgtcatcaacggagccctctatctagcagattctgtgcttgccttcattcattacactaaatatgcctaattttagaatacaccataagtgaacattgctgacattgtttagaatgtagtgtttgtacgattagtgaggctaaggaatttattataattacttagattcatatactttggtaactattatacaagagaacgtaatccttaaggcattgaagcgtagctctatgcttcctcataatatccgcccttttcatgcaaaagttcaatcccacaacaacaatagccttgcaacaataatgttcaaatgattcctataaatgacaaataaatgaaaaggtaggaattaccaccgtacc

SEQ ID NO: 54 - DNA, 939 bp, Aedes aegypti
atgatagcactgaacctggtggttctcatcatctaccgaacgggctacgggggtgatttcctcggcgtgggtggcacctggaacctgaacgaggagaaaagtcccgacgcggaaattgtggcctccggagtgtttgtcggctacttcatctacacctcggtgcagctgattacgttctgcttcggcacgaccaagctgaagcgggagctttcggacactataatgaatgtggtcggtacgttcatgtgggtcgccgtcggaggcacggccctccactattggcacggttatctggcggaacatgacttccagcacatcacctcggagagaacggctggacttgccctgggatcgctttgcgttgtgaccggtgccctgtacctggccgattcggtgctggccttcatccactacgccaagcacgagaacagcaagtactaattgaacgcgaacggcggcccgaagaaaagtaccgatccaggtgtggtaggttgtaagcttgaattattgcagcgggtgctatcaagattttgccctaaaatgtgcagcaatgcttccgtgtactagttaataatgtaagtaagtaaggtctaaacgaagaggaaaaactgtcataatttaggtaccgtaagcgtggttttaagataaaaagagcagaggtgaaacttttacctacttaatgcttattgcttggtgggaggcacatttgccaagataaatggcaagtaacgacattttctgttagtattctacaaatatacataacaatacaaatgtaagcgataagtaagaagccgtgaaagtgcaacgtaccgaattccttaacgagaaaagaaatagaaccggacaattcgatgtaaaatataaataccacatttaagaaagttgtatccaagaaaagattagaatttcattaccaataaaccgcatttcttatgg

SEQ ID NO: 55 - DNA, 1002 bp, Dendroctonus ponderosae
ggggacttatctctgtttcacctcaatctagacgttcgcaggtgcttccatctgaacaaaattcacttttcattgctttgaaatttccaactttccctcactttagtgccgctggttcgtcaaaatggccttagagacgatagcctccattatagttaaactagtgaagctggtattgaactttatcatcctggttctctatcgcgtgggcttcgctggcgacttcttgggagtcggaggaacatggaacttgttcgaggagaagagctcagacgtggaaattatcgcctcaggagtttttgtggggtatttcgtctacaccgcagtgtccctgatcagtctctgccttgccagcagcgaaaataaaaacactttcacggatattttgatgaacattgtcggagtatttctttggatcgctgtgggagccacagctttgcactactggcatggttatcttagtgagcacaagtacacttatgtgaactcagaaagacaggttggtctcgctttggggtccttaagtgtgctcaatggtgcagtttacctcgttgatagcgtcatatcagtaatattcttaataaaagccaagttgcagtagatgttgaaaacattttcacaatcactctccacttctgtttcgtgttcagtggccggtttcatttggaattgaaacacagtcagaaaagtacgtacaatatacgtataaggtaaagttcatggaaaaatttttaaatctgtttttacgcttactaattacaagtacaaatgaagaaacatatgtattacattattacatgacagactattttaatcttgcaggctgaaaggcttttatggattttgccattttttgttaaataattactatgttatggatgtacttgttttaccaataaatgtagtttgaggttgtagaatagtacctaaataggcttaagtggctgtttttacgaaaagaaatatatcaagagcaaataatcagtaaaaaaaaaaaaaaaa

SEQ ID NO: 56 - DNA, 468 bp, Pediculus humanus corporis; identical to SEQ ID NO: 26

SEQ ID NO: 57 - DNA, 80 bp, Nezara viridula
gggggagtattatctctgtaacagattattgtttcatttttgtgaggtgaaaaaaagtattaacatggacatccaaacta

SEQ ID NO: 58 - DNA, 1080 bp, Lygus hesperus
attttaccattcgctgctgcgtttcttgtagaggtgtaaaaatgccaagacggtatccaacagaaaaactgctccttccaccacacataagcttccaagcgcgattcctacttgttttgtcgtggatatggattgtgcgtggttggcgttctggtatccgcaccaatagtgcagagctatacctccgacgccaatgaacatgatgattcctgttatgttcattatgaagtccacgacgctcgctttgtgtttagtcgttccgaagcaaaatgctattaggatgactgctgtgtagatgaagtagcccacaaaaactccggaggcaactatttcgacgtctgggtttttcacttcgtttagattccacgttcctccaacacctaagaagccgctgtttccaccgagcagacgaaatggagttggaaaccatcatcacaattttcatcaaagtcctcaaattggtgctggacttcataataatgatgatgtaccgctacggtggaaacagcggcttcttaggtgttggaggaacgtggaatctaaacgaagtgaaaaacccagacgtcgaaatagttgcctccggagtttttgtgggctacttcatctacacagcagtcatcctaatagcattttgcttcggaacgactaaacacaaagcgagcgtcgtggacttcataatgaacataacaggaatcatcatgttcattggcgtcggaggtatagctctgcactattggtgcggataccagaacgccaaccacgcacaatccatatccacgacaaaacaagtaggaatcgcgcttggaagcttatgtgtggtggaaggagcagtttttctgttggataccgtcttggcatttttacacctctacaagaaacgcagcagcgaatggtaaaattgattgtgacaaactatcgagttcgggaaaacaatattaatttctttgtaaataaaaacctcctggctttgggggcatgggctggtacgtacgaaggaccactagcaggttgggagtggtcttcttccgtgtccacaataaaggcacggggatgtagattataacacatcgtagatgttat

SEQ ID NO: 59 - DNA, 1237 bp, Euschistus servus
gggaaaatgagtaatctcataaattacacaaactgataagatagtatattatctcttttaaaaaagattattgtttcattttggtgaggtgaaaaactattaacatggatatccaaactattgcaacaatatttattaaagtactaaaacttgtaattaacttcatcattttgatgctttaccgctatggtggaaagcaaggatttctgggggtaggtggaacatggaacctgtatgaagtaaagagtgcagacgctgaaattattgcatcgggtgtttttgtaggattttttatatatacatctgtcattttgatttcctactgcttcggaacaacaaaacacaaatacagtgccgtggacataataatgaatgtgtgtggaactatactgtttactgcagtaggaggaatagcattacactattgggtcggcttccaggatgaaaaccattacgtgggaatctctcaagaaaaacaaattgggcttgcactgggcagcctttgcgtcgtctgtgcagctgtctatctcctagatacagttttatcttgtattcatatcgcccacaagcatgctattgcttaattttatttaaagcaacttcacagcatgaaaagacttcccaaatcaagtaatgagcgaatccagtttataatatgtataacagttgtaagatgaggataagttaaaattgataatgtaatttttggatttaaggttaaacgatgttaaaatatataaatatgactttccttggtaagcagaacaaagaacctaagcaattaaataaaaaacatgtatcatacgacatgttcttagctgcatatataaatattagttcagaggtactattaatatatgcatagtggtcaccgggaataatagaataatatttgatttgaagcactacaaagatgttttaatacttgatattttagtccaataaaattattttgttcttataacttttattaacaggaacagttatatagtaaaaaaaagttctgacctcaaatatcatgattttatacgtaaaaacttattccctgtgagaagagataaatgtacatgacttcgcctaggtttaaatctcaattcctacgtttcaattacattcagttctattaaatatatattttagaacatactatagctcatagctctatgaaatgtagtaattattattttttaagtccttaaaattaatttaatcatgtgatttatgttttagctgttat

SEQ ID NO: 60 - DNA, 874 bp, Halyomorpha halys
tatcggtatggtggaaaacaaggatttctaggagttggtggaacatggaatctgtatgaagtaaaaagtgcagatgctgaaatcattgcatcgggagtttttgtgggattttttatatatacctctgtcattctaatttcgtactgtttcggaacaacaaaacacaaatatagtgctgtggatatcataatgaacgtaagcggaactatattgttcactgcagtaggaggaatagcattacactattgggtcggcttccaggatgaaaaccattacgtcggcatctctcaagaaaaacaaataggacttgcactaggcagtcttagcgtcgtttgtgcagctatttaccttctcgatacagttttatcatgcatacatattgcacacaagcatgcaatcgcttaattttatttaaagtatttatacagcacaaaaagacaaacgaagattatgacacatagcagttgtgtaaatgagttttttaaaaatttaataatgaaatttgtgtatcaggaactacttctttaataggatttatgaatagaacattattcttagctaaaaaaaaaaaaaaacactggttcagatttggtgagaaatgaaaattagagatatatacataataatgcttgtagctttatgagccatgatttaaagttttacatttatgaataaaaaatattgccttagttggaaatctcaattcaagttccagcgaatgaatgtaccttttataaaatgcaatgtttcatatctgtttgaaaggtagcagcaaataatttctaaagtcaaattatccaattaattttttattactttttaaattattaattttatttcctttaatttacaccagttttctataatattgaaa

SEQ ID NO: 61 - DNA, 1263 bp, Megacopta cribraria
ttcaggtaaagtaaaaaaggttttttttgatatggatatccaaactatagcaacaatttttattaaggtcttaaaacttgtaattaattttattattttgatgctctatcgatatggtggaaaacaaggatttttaggaataggaggaacatggaacttgtatgaggtgaagagtgcagatgctgaaataatagcatcaggggtctttgttgggttttttatatacacatcagtactgctaatatcctactgctttggaacaacaaaacataaatacagtgcagtagatatgataatgaatgtaagcggaactatattatttaccgctgtaggtggaattgcattacactactgggttggatatcaagatgaaaatcattatgtgggtatttctcaggaaaaacaagtaggattggccctaggaagtctgacaattgtttgtgcagctatttatcttctggacactgtcctatcttgtatacatattgcacacaaacatgctattgcctaactttaaaaccaatttgttttttttatctgtaaaaaaaaaagaagaaaaaaaagacattaggttagaatattctactgtcaaagttttcaaacacccaatcatttgttgtttcattattataagtatatttcagtttagaaagatattacatttttttttttttaatttagattatagttttttttttaagtagttctataatattcaaacctaaaataaatcacatcaaaaaatttactgtcacaaatttttttttttcaaaaagtcaaatctgattatggtcagtaccagaaagaaggaaaaatttggttttttattggttgttagaaatggaccaccgattttttgtcaccccccccccctaacatttcaggcgatatttttaacctagcttgtcacctttatattattgatgaatatttaaaatgtctatgaaatataaaagatagatttgaacatgaagaatgctaattttatattcgaaagacaaaaacccacactttctatttcctgaaacgtttgcttaattgaaacatatttgagtgacatcaacttactttaaaatttatttttaaatgaaacttgctatttacagttttatttattacttgtaatgttaatttctaagtccatagattaattaatcattataacaatgtaatttgagtaaagctagttagttaccttgttaataaaattattatagacatttcatagattatcatttaaatttgtcacatgcaatttcatat

SEQ ID NO: 62 - DNA, 1445 bp, Manduca sexta
aatccatcacaattgtggttagggagtaaataataaaaacaaccattacctaagcaagtcaccacaaagaagtacacgaagcatgtcatgaatgaacttgtttaatcttgttttactgttttcactttacttaattgtgtcatgtatataacaatcaaatataactatttctctttgaaattatcatgtttaaaacaagaatatgatggttgtgaaaatagtttagatatatgttatttagtatcctggggaacgttgatctgtacttaattaaaaaaaagataaaattgtaaacgttcgctgtactttatagtttgtaaacgtagtgagagagaaagattaatgccaatacatgatgcttatacgcgagttactacaaataccattcccggattttaaacacagaaacctaattatcacaccaaatagaatagcctaaataaaaatagcaattcagtcaacaccaaaaaccaacacacaaaatgaaccttacttatttatcaacagagtccagttttactgtaacaccaataaaagtacattgcaacctgccctagtaattcgctgaaacagataagattaggagctccaactatgctgataaagcgatacaaaactgataaacgaactcacactgtgaaggtccagtcgacaggtactcgctgtagacaaagaagtggtgtgatcgtgaattttgagattaaagtgtcaaaaatattgtgaaaatggtgtccgttcagacgatagcgacaattgtcgttaaagctttcaaaattgtattgaacataattatcctggtgctctaccgaactggctacaacggcgagtttctcggagtgggcggtacatggaacttgaacgaagaaaagaacccagacgctgagatcgtcgcgtcaggagttatcgtcggctacctgatctacacgctggtgcaagttgttaccttcctgtttggcaccacggagcataaacgcgccatgtctgagatcgtgatgaactttgtgggcgtgttcctgtggatcgcggtgggcgcggtggcgctgcactactggggcgggtaccagggcgagcaccagttccaattcgttttcgctgagaaacaagtgggtctcgcagtgggtgctctctgtgtgatccaaggcgcagtgtacctcttagatacagcactatccgtcatacacttcactaaggagatgtaaatttaagtgcatttaattcgttaatcatagttttaaacctttgagtcgaacgagaagtttccaaataaacgaataacattgtaaggttagtaagttataataataaaaaaccaggtgaaattatattctatttccaaaagtttgaatttcgaatgtcatcaattgtctacaaattacgagcaaaaataggaaaaaataattagctctcaacaatcttcttcgttttcccacctcttcttagttaag

SEQ ID NO: 63 - DNA, 880 bp, Euschistus heros
tattgtttcattttggtgaggtgaaaaaaactattaacatggatatccaaaccattgcaacgatatttattaaagttttaaaacttgtaatcaattttatcattttgatgctctaccgctatggtggaaaacaaggatttctcggagttggtggaacatggaacttgtatgaagttaaaagtgctgatgctgaaattattgcatctggagtttttgtgggattttttatatatacatctgtaatccttatttcatactgctttggaacaaccaaacacaaatatagtgcagtagatattataatgaacgtgagcggaaccatcttgttcaccgcagttggagggatagctttacactattgggttggcttccaggatgaaaatcattacgtgggaatctctcaagaaaagcaaataggacttgcgttgggcagtcttagcgtagtttgcgcagctatttaccttcttgatacagttttatcttgtatacatatcgcgcataagcacgcgatcgcttaatttcattcaaactactaagtacagcataaaaagacacgataaataaagtattgaggaaggtaagactatgaaaaatagtagaagtgaggatttaaataaattaaaaaatgtaatctatccaaccaagtctaatttttattaaataaagttttcatatgaataatgaaaagaaaatacttgtttaattctagaaagaaatgaaatcaggtaaataacaaagctatgtaaaaaaaaaaactacttagattaaaacatacaattacagtaaatgaaggtgctttttattaagaaaataatataattcatggctgtttatatgcttatgttgtgtcagttgcagtgatctacttgttaaatagtgc

SEQ ID NO: 64 - DNA, 1243 bp, Piezodorus guildinii
ctctttgaaaaaagattattgtttcattttggtgaggtgaaaaactttttaacatggatatccaaactattgcaacaatatttattaaagttttaaaacttgtaattaatttcatcattttgatgctttaccgctatggtggaaagcaaggatttttgggggtaggtggaacatggaacctgtatgaagtaaagagtgcagatgctgaaattattgcatcgggtgtttttgttggattttttatatatacatctgtcattttgatttcctactgcttcggaacaacaaaacacaaatatagtgcagtggacataataatgaatgtgtgtggaactatactgtttactgcagtaggaggaatagcattacactattgggtcggcttccaggatgaaaaccattatgtgggaatctctcaagaaaaacaaatcggccttgcactgggcagcctttgcgtcgtctgtgcagctatttatcttctagatacagttttatcttgtattcatatcgcccacaagcatgccattgcttaattttatttaaagcaacttcacagcataaaaagacttcctaagtcaagtaatgagtgaatccagtatatgtataacagttgtaagatgtggataagctaaaattgataatgtaattctcgatttaaggttaagcgatgttaaaatatatcaatatgactttatgtggtaagcataacaaagaacctaagcaattaaataaaaaacagctaaataattagctacatatataaatgctagttcagagatactattaatatgtgcatagtggtcactggaataatagaataatatttgatttgaaacagtataaagatatttttaactcacggttttaatacctgatattttagttcaatcaaattaatttattcttataactttatttacaagaacagttcaatagtaaaaaaaaggtctgctctcaaatatcatgattttatatgtaaaaacttattccccatcagaagagataaatgtaaattacttcgccaagctttaaatctcaatttctacgtttcaattacattcagctctattaaatatatactttagattatactatacctcatagctctatgaaatgtagtaattattattattttttaagttctttaaattaatttaatcatgtgattaatgttttagctgttatcaaatacattttttttttgagggggggggtcgtcaaaaaattactatttttcaaataa

SEQ ID NO: 65 - DNA, 797 bp, Tribolium castaneum
acgcgtccgcacaggtggagaaactgtaggtgtgcgaagctagtgcgtttttttgtgtcccaaagggcgaattccgacaatgacgagcattgaaactgtgggggccctaatcctcaaaatcctcaagttggtcctcaacttgatcatcattatcttgtaccgcaccggctttagcggcggttttctgggagttggaggcacttggaacttgaacgaggagaaaaacccggatgctgaaatcgtagcatcggggattttcgttggatattttatttacacatgcgtttcgatcataagtttatgctttgctacggccgaccacaaaaacacttttaccgacattttgatgaacattattggggtttttctgtgggttgccatcggggccactgcgattcactactggagtggttacctcttggaacataagtaccaaacaacggcttccgaaagagaggtggggctggctatgggggccttgtgtatactgaatggagcggcgtacctcatcgatacagtcttgtctgtcatttttgtcatcaaagcgaaattgtaaacgggtcaagcattgactgccgagtatgacaactgttcgaagtagatttagttttacacaagttatgctgttagattaagttcaaattttgtaataagtttcacttcatattagatatatttatgccattgttttttatgtacgtaactaggaacttccctcacatttttaataaagtgttgacgcaaaatgtaaaatgtgttggaaagtttcactgaattaataaagatatgccttttta

SEQ ID NO: 66 - DNA, 3948 bp, Diabrotica virgifera virgifera
atgtggttcaagtactttgttatttttgttttgtttagtgttagcgttaaaggactagatggtgatcttccaccagatgtcgaaactatcgatgtggagactacgaatgtaagtaacgcggatccagacgcagggaaacctattccggtggaagatacgcctaatggagtccctccagtgaatacaaactatgatcctatgacctcagacacagctccaccagacactgaccaacgaggaggaggaggaactccatacactatatcagaaccgagactggcagaaataaggaaacattttatgtacccatttttcgataaaggaggcagtgataataacttgggagatttccaaaaggacattcaatcttccattcctcaggtgcacaagaatttaaacttccagttgcctttctttggattcaggtataattacacaagagtttcagtgaatgggtatctggaattcagcgatccacctccaaattatgactatccattagtatttccaattaaagaatggcccaaaaagaatgatcctgcctttattggcattttcttcagcagatgcagaatcggtaatctacgtgatggagacattgatcaaagaacacccggcgtctattttagaatggaaagagatttaagaacgaggcaagacagaatgggtgtagaaattagagaaagacttaagtgggacatcagaacaggagtcattggatcagaaacattcgatcctaagcacgccgttattgtcacttggaaaaatgttactttcactggaggattcgctaatgctaaatacaagacaaacaccttccaaatggttcttgcaactgacgaagtcttcacttacgcgatgttcaactacctcaacttggactggacctcccacactgaagcgggtggtgattcccaaaacggagaaggtggtgtcagtgcctatgttggttttaacgctggaaacggtaccagaagctacgaatacaatccttacagtcaagcttccgtcattcgagatctcacatcagttggatttggaaatggttttaagggaagacatattttccgtatagacgaagacattttgcttggatcgtgcaacaaagatatagatggtgccaaccttcccctgaaattcgctcctgaaagtggtaacatgttgggtggaacagtggtaaatataactggtccatgcttcaatttgaatgataggattcgatgcaagtttgacgtatcaaatgaagtatttgggtatgttgttgataaaaacagagctatctgtgtacaacctcaactttatgccgaaggatgggtgaatcttcaaattgctgttaattccgaagcattcaagtggaaggggaagtattatgtagaatcacctgctagtgcgactcaaaaaatcttctttaaggacatgaaattacacgaaaaatctccgagcgaattaaaaatcacttgggagaagcagaatttgaccacaaacgacaacgctaacatccgaatttctctctggggttacagagaaacgactatgaaaccagtgtttgtttatatcaccgatatcgctgataatgttcaaaacattggagaacacacgattgttccttctcagtttagaactagagtcaatcaatatctgactgatattaagtttggattcctccaaattaatttaactgagtctatcacagttaatacatatactacaagccagagcagcataaatttaacaccggtaatatggagccgacccattccactcggttggtacttccagttccaatgggaaaaccagtacggtaaaaattggcccaaatatctatgtgacgattggctgagaacggacagatatctaaaaaacttcgcccacgaacttgctcaatgtccctgcaccgtcgaacaggctttagctgacaagggcaggtttatgccagacttcgattgcgacaaagattcgaacccgatttgttactataacaaccaagcacttcattgtgtgaaaactggatctcccactttagaaggctccgaacaacaatgttgctatgataaaaacggctttttgatgctctcttacgatcagcaatggggttctagtccgagacgttgccacaacttgggtaaaatgccttacaatgaagcgacaaaagtgcctactctctcacaatggtttaatgacatggtacccaaatatttgtgttgtctttggcaagacgagcaagctgtgggatgtgaaacactgagatttgaacggagacctacacaggattgtgtagcttaccaagcaccaggtgtagcaggagtctacggtgatccacactttgttactttcgacgatgtagaatatacgtttaatggaaagggagaattcgcattggtgaaatcagttacgcaaacggacaatctggaaatccaaggaagatttgagcaaatggaccctaacacttatggtgaggttagagctactcagctgacttctgtagtagctaaaggtaataacacaatagcaattgaagtcagaaggagacctctggattctagatggagatataggttggatgttatagcagataatagaagactttattttgatagaccctcactaaaattccagcacttccaaggtgttactatttacactccaagctacatccttaatcaatcagaagtcatcattatgtttgacaatggagctggaatggaggttgtcgacaatcaaggattcatgagtgcaagagtatttttgccttggtcgtttataaataaaaccataggcctttttggaaattggagttttaacaaagaagatgacttcacacttccagatggttctagagcagcaatagtgaataacataaatgacatggaaagggtttacaatgattttggttccaaatggatgttggaggatgtgttagatcctcaaaagggtagagcattgttccacagggaattcggaagaacttcagcgacttacaacaacaaaacgttcaagcctcaattcctaatgaatccggaagatttcctaccaaccaacagatctacagatcttaagcgaatatccgaaatatgtcctctaaagttgtacgaatgttactacgattacgctatgacggttgacagggatttggcgcattacaccaagaattataaagccacgatctatcaatataaagaaacgacaaggaggaaagttgtgtcttgcggagtactagaaacaccgagattcggacgaaagagtactttcctgttcgtgcccggtacgaaggttacctatgaatgcatccaagagttcgtgttggtaggtgatcagcggagagagtgtcaagcggatggaacatggaatattcctgaatacggatacacatattgcttacgtcaagaagaatattcttcgcgtcaagctgccatcacttcaggtataatcctcgcaattatcataccattggttcttctattaggttacgttggctacaaaatatacgaaagactaacaagtaacaatcaaaactatgactacgacaccgtacaaaagactcaaaatctccaacaattcagtcgaactttaagtccatacagagaagaagacgaggacgaaaagtatcctcaaagcgacactgattcactaagcaagaaacgaagaagttacgacaaatcctacagaactcacgaacctttgcccaatagaccaaattctgagtttgaggaaaaaccattggacccttacgatcaaaccttcgaagacgacagggtttctagaacgcctacgggaagtccaaccagccctacttctagtatacaatatacaacgccttatagtcggccagacattattagagggaacagctcaaagaatttgaactataacgacatgtatgcagagccaataaagaaacctaaagaaaggattagcgctagtcagagtagtatagtgacagacgtttag

SEQ ID NO: 67 - DNA, 3771 bp, Diabrotica undecimpunctata howardi
atgtggttaaagtactttgttatttttgttttgtttagtgttagcattaaaggacaagacgatggacttccaccagatgtagaaactatcgacaccaatgtaagtaacgcggagccagatgtagggaaacctattccggtggaagatacgcctaatggagtgtctccagtgaatccaaactatgaagtagataaatccctagttaatgtaagcaatagtttggtaaatgtgactaaaaatcccagacggacttatcagacagttaacaaactgggcaaatattcgacattttatgctgcagattacgatcctatgacctcagacaccgctccaccagacactgaccaacgaggaggagggactccatacactataaccgaaacgagattggcagaaataaggaaacattttatgtacccatttttcgataaaggaggtagtgataataacttgggagatttccaaaaggacattcaatcttccattcctcaggtgcacaagaatttaaacttccaattgcctttctttggattcaggtataactacacaagagtgtcagtgaatggatatctggaatttagcgacccacctccaaattatgactatcctttagtatttcctattaaagaatggcccaaaaagaatgatcctgcctttattggtatattcttcagcagatgcagaatcggtaatcttcgtgatggagacgtcgatcaaagaacaccaggggtctattttagaatggaaagagatttaagaaccaggcaagacagaatgggtgtagaaattagagaaagacttaaatgggacatcagagaaggcgtcattggatctgacaccttcgatcctaagcacgccgtgattgtcacttggaaaaatgtttccttcactggaggattcgctaatgctaagtacaagacaaacaccttccaaatggttcttgcaactgacgaagtattcacttacgcgatgttcaactacctaaacttggactggacctcccacactgaagccggtggtgattcgcaaaacggagaaggtggtgtcagtgcctacgttggttttaacgcgggaaatggtactagaagctacgaatacaatccttacagtcaagcttccgtcattcgagatcttacttcagttggatttggaaatggttttaagggaagacatattttccgtattgatgaggaaattttgcttggatcgtgcaacaaagatatagatggtgccaaccttcccctgaaattcgctcctgaaagtggtaatatgttgggaggaaccgtggtaaatataacaggtccatgcttcaatttgaatgataggattcgatgcaagtttgacgtttcaaatgaagtattcggttatgttgttgataagaacagagctatatgcgtacagccccaactgtttgccgaaggatgggttaatcttcaaattgctataaattctgaatcattcaagtggaagggaaagtattatgtagaatccccttctagtgcaacccaaaaaatcttcttcaaggacatgaaataccacgaaaaatctccgagtgaattaaaaataacttgggagaagcaaaatttgaccacaaatgaaaacgctaacatccgaatttccctctggggttacagagaaacaactatgaaaccagtgtttgtttatatcaccgatatcgcagatagtgttcaaaacattggagaatacacgattgttccttctcagtatagaactagagtaaaccaatatttgactgatattaagtttggattcctccaaattaatttaactgagtctatcaaagttaatacatacactacaacccaaacaaccatagaattaacaccggtagtatggagccggcccattccactcggttggtattttcaattccaatgggagaaccagtacggcaaaaattggcccaaaactctatgtgacgattggctgagaacggacagatatctaaagaacttcgctcacgaacttgctcagtgtccctgcaccgtcgaacaggctttagctgacaagggcaggtttatgccagatttcgactgtgacaaagattcgaatccgatttgttactataacaaccaagcacttcattgtgtgaaaactggatctcccactttagaaggttctgaacaacaatgttgctatgataaaaacggctttttgatgctctcttacgatcaacaatggggttctagtccgagacgttgccacaacttaggtaaaatgccttacaatgaagcgacaaaagttcctactctctcacaatggtttaatgatatggtacccaaatatttatgttgcctttggcaagacgagcaagctgttggatgtgaaacactgagatttgaacgtagaccaacacaagattgtgtagcttaccaagcaccaggcgtcgcaggagtctatggtgatccacactttgttactttcgacgatgtagaatatacgttcaatggaaaaggggaattcgcattggtgaaatcggttacacaaacggacaatctggaaattcaaggaagattcgagcaaatggaccctaacgcttatggtgaggttagagctactcagctgacatctgtagtagctaaaggtaataacacaatagcaattgaagtcagaaggcgaccactagattctagatggagatataggttggatgttatagcagataatagaagactgtatttcgatagaccctcactaaaattccagcacttccaaggagttactatttatactccaacctacatccttaatcaatcagaagtcatcattatgtttgacaatggagctggaatggaggttgtcgataatcaaggatacatgagtgcaagagtatttttgccttggtcgtttataaataaaaccataggcctttttggaaattggagtttcaacaaagaagacgactttacacttccagatggttccagagcagcaatagtctctaacataaatgacatggaaagggtttacactgattttggttccaaatggatgttagatgatgtgttagatcctcaaaagggtagagcgttattccatagggaatttggaagaacttcagccacttacaacaacaagacgttcaagcctcaattcctaatgaccccggaagatttcataccagccaacagaactgcggatattcagcgaacatacgaaatatgtcctctaaggatgtacgaatgttactacgattacggtatgacgcttgatagggatttggcacattataccaagaattataaagccacaatatatcaatataaagaaactacaaggaccaaagttgtgtcttgtggtgtactggaaacaccgaggtttggacgaaagagtactttcctgttcgtgcctggaactaaggttacctacgaatgcattcaggagttcgtgctggtaggtgatcagcgcagggagtgtcaagcggatggaacgtggaatattcctgaatacggatacacttattgcttacgtcagcaagaatactcccaacgacaggctggtctggcttccgggataattatggcaattcttataccgctcatcttgcttttcgtctatctagcgtatcgtttcttacaaaaaaagcagaaagagaaagaagctgaaaaattagaagagcagcaatttatggatcaacaaagaagggcacacgaagcagctactaagaagttaacagaagccgaatatgcttcagaagatgaagacagtaatgttacaagcacgggcgcaaaagaaacaactgtatattaa

SEQ ID NO: 68 - DNA, 4068 bp, Diabrotica barberi
atgtggttaaagtactttgttatttttgttttgtttagtgttagcgttaaaggactagatggtggtcttccaccagatgttgaaactatcgatgtggacactaccaatgtaagtaacgcgaatccagatgcagggaaacctatcccggtggaagatacacctaatggagtcgctccagttaatccaaactatgaagtagataaatccttagttaatgtaaccaatagtttggtaaatgtgactaaaaatcccagacggacttatcagacagttaacaaactgggcaaatattcgacattttatgctgcagattacgatcctatgacctcagacacagctccaccagacactgaccaacgaggaggaggaactccatacactataacagaaacaagactggcagaaataaggaaacattttatgtacccatttttcgataaaggaggcagtgataataacttgggagatttccaaaaggacattcaatcttccattcctcaggtgcacaagaatttaaacttccagttgcctttctttggattcagatataattacacgagagtttcagtgaatgggtatctggaattcagcgatccacctccaaattatgactatccattagtatttccaattaaagaatggcccaaaaagaatgatcctgcctttattggtattttcttcagcagatgcagaatcggtaatctgcgtgatggagacattgaccaaagaacacccggggtctattttagaatggaaagagatttaagaacgaggcaagacagaatgggtgtagaaattagagaaagacttaagtgggacatcagaacaggagtcattggatcagaaacattcgatcctaagcacgccgttattgtcacttggaaaaacgtttctttcactggaggattcgctaatgctaaatacaagaccaacaccttccaaatggttcttgccactgacgaagtcttcacttacgcgatgttcaactacctcaacttggactggacctcccacactgaagcgggtggtgattcccaaaacggagaaggtggtgtcagtgcctatgttggttttaacgctggaaacggtaccagaagctacgaatacaatccttacagtcaagcttccgtcattcgagatctcactgcagttggatttggaaatggttttaagggaagacatattttccgtatagacgaagacattttgcttggatcatgcaacaaagatatagatggtgccaaccttcccctgaaattcgctcctgaaagtggtaacatgttgggtggaacagtggtaaatataactggtccatgcttcaatttgaatgataggattcgatgcaagtttgacgtatcaaatgaagtatttgggtatgttgttgataaaaacagagcgatctgtgtacaacctcaactttatgccgaaggatgggtgaatcttcaaattgctgttaattccgaagcattcaagtggaaggggaagtattatgtagaatctcctgctagtgcgactcaaaaaatcttcttcaaggacatgaaattacacgaaaaatctccgagcgaattaaaaatcacttgggagaagcagaatttgaccacaaacgacaacgctaatattcgaatttctctctggggttacagagaaacaactatgaaaccagtgtttgtttatatcaccgatatcgcagataatgttcaaaacattggagaatacacgattgttccttctcagtttagaactaaagtgaaccaatatttgactgatattaagtttggattcctccaaattaatttaactgagtctatcaaagttaatacatatactacaagccagagcagcatagaattaacaccggtagtatggagccgacccattccactcggttggtacttccagttccaatgggaaaaccagtacggtaaaaattggcccaaatatttatgtgacgattggctgagaacggacagatatctaaagaacttcgctcacgaacttgctcaatgtccctgcaccgtcgaacaagctctagctgacaagggcaggtttatgccagacttcgattgcgacaaagattcgaacccgatttgttactataacaaccaagcacttcattgtgtgaaaactggatctcccactttagaaggctccgaacaacaatgttgctatgataaaaacggctttttgatgctctcttacgatcaacaatggggttctagtcccagacgttgccacaacttgggtaaaatgccttacaacgaagcgacaaaagtgcctactctctcacaatggtttaatgacatggtacccaaatatttgtgttgtctttggcaagacgagcaagctgtgggatgtgaaacactgagatttgaacgtagacctacacaggattgtgtagcttaccaagcaccaggtgtagcaggagtctacggtgatccacactttgttactttcgacgatgtagaatatacgtttaatggaaaaggagaattcgcattggtgaaatcagttacgcaaacggacaatctggaaatccaaggaagatttgagcaaatggaccctaacacttatggtgaggttagagctactcagctgacttctgtagtagctaaaggtaataacacaatagcaattgaagtcagaaggagacctctagattctagatggagatataggttggatgttatagcagataatagaagactttattttgatagaccctcactaaaattccagcacttccaaggtgttactatttacactccaacctacattcttaatcaatcagaagtcatcattatgtttgacaatggagctggaatggaggttgtcgacaatcaaggattcatgagtgcaagagtatttttgccttggtcgtttataaataaaaccataggcctttttggaaattggagtttcaacaaagaagatgacttcacacttccagatggttctagagcagcaatagtcaataacataaatgacatggaaagggtttacaatgattttggttccaaatggatgttggaggatgtgttagatcctcaaaagggtagagcgctgtttcacagggaattcggaagaacttcagcgacttacaacaacaaaacgttcaagcctcaattcctaatgaatccggaagatttcctaccaaccaacagatctacagatcttaagcgaatatccgaaatatgtcctctaaagttgtacgaatgttactacgattacgctatgacggttgacagggatttggcgcattacaccaagaattataaagccacgatctatcaatataaagaaacgacaaggaggaaagttgtgtcttgcggagtactagaaacaccgagattcggacgaaagagtactttcctgttcgtgcccggtacgaaggttacctatgaatgcatccaagagttcgtgttggtaggtgatcagcggagagagtgtcaagcggatggaacatggaatattcctgaatacggatacacatattgcttacgtcaagaagaatattcttcgcgtcaagctgccatcacttcaggtataatcctcgcaattatcataccattggttcttctaataggttacggtggctacaaagtatatcaaagactaacaagtaacaatcaaaactatgactacgacaccgtacaaaagactcaaaatctccaacaattcagtcgaactttaagtccatacagagaagaagacgaggacgaaaagtatcctcaaagcgacactgactcgcttagcaagaaacgaagaagttacgacaagtcctacagaactcacgaacctttgcccaatagaccgaattctgagttcgaggagaagccattggacccttacgatcaaaccttcgaagacgatagggtttctagaacgcctacgggaagtccaactagtcctacttctagtattcaatatacaacgccttatagtcgaccagacattattagagggaacagctcaaagaatttgaactataacgacatgtatgcagaaccaataaagaaacctaaagaaaggattagcgctagtcagagtagtatagtgacagacgtttag

SEQ ID NO: 69 - DNA, 3843 bp, Leptinotarsa decemlineata
atgtacgtcaagtggaaattggttttgactctagtactgtgtgttggtgtcgtgattggggaagatatttcaacagatataatccccttgccacaagacaatacagcagatgtggaaatagtggctacagaaactaggtcaaattcaggatctgaagctgctgtagaagagcccaccaataacactgggccttcagatactacaaacccaaattatgctgtagtgccccctgtagtttctgatagcaaacctgcaataacaaatagtgaaacaaatgacactaacaatgatgttgtaatgttgagtcctacgaaatttctcatgaaaaagggacgatctggacgtttattggaatatccgactgattacgatcctatgacttcccatattgctccaccggatagtgatcagagaggatattcaggagtaccatatgtcttgacggaaaccagactgcaacagatccgccaaaatttcatgtatccctactacaacagaggcggtaatgcagatgacgaaggagactaccagaaagaaattcagtcatctattccgcaagtgtacaagaacctcaacttccaactccctttcttcggatttcgattcaattacacgagggtctccttaaacggctatttggaattcagcgatcctcctccaaattacgactatcctttggtctttccagtcaaggaatggcctaaaaagaacgacccttctttcatcggtatctttttcagtaaatgtagaatcggtaacctgagggacggagatattgatcaaagagaccctggagtgtactttaggatggaaagggatctcagaaataggcaggacaggatgggagtggagatcagagaacgactgaaatgggatataagggaaggggtgatagggtcagaaacattcaatcccaaacatgccattatagtcacatggaaaaatatctctttcaatggaggttttggcaatgctctctaccagactaacactttccaaatgatcctcgccactgatgaagttttcacctacgccatgttcaactacttgaatcttgactggaccacccacactgaagcgggaggcgacacaagaaaaggagaaggaggagttcccgcttttgtgggattcaacgctggaaacggtactagaagttttgaatacaaaccatacagtcaagaatctgttattcgagatctcacacaaactggtttcgctaatggtttcaaaggaaggcacattttccgaatcgacgaaaatatcctaactggaacatgcaataaagatatagatggtgctaatctaccgctaatgatatctccagaaagtggaaatatgctgggtggaacaatagtgaatataacaggaccttgtttcggcctagacgaccaagttaaatgcaaatttgatgtagcaaatgaaataaatggcgtcgttatagataaaaacagggctatatgcatccaacctagactgtatgccgaaggatgggtgaatttacaaatagccataggggctggggtatacaaatggaagggaaaatattatgtcgaatcccccgcagcggcatctcaaaaaatctacttcaaggacatgaaggttcatgaaaaatcgcctagtgaaataagaataacttgggaaaaatacaacttgaccactaacgaaaacgctaacattcgtatctccttatggggttacagagaaacaacaataaggccaacgttcgtttacatcactgatatcgcagacagtctccaaaatactggagagtataccatcgtaccgtcccaatatagaacaaaagttaatgagtttctcacggatatcaaatttggtttcttgcaaattaacttgactgaatctatcaaagtgaacacttatacatctgtacaaagatcggtggaaatagttcctgttgtatggagtcgacccattcccctaggatggtactttcagttccaatgggaaaatatgtatggacgaagctggcccaaagcactctgcgatgactggctaagaacagacagatacctgaaaaactttgctcatgagttacctcaatgcccttgcactgtagaacaggctttggcagacaaagggaggtatatgcccgactttgattgcgacaaggactcaaatcccgtatgctactacaataaccaagctctgcactgtgtgaaaacaggatcaccaacgttggagggatcagaacagcagtgctgctatgacaaaaacgggtatctcatgttatcatacgatcagcagtggggttcaagtccacggcgttgccacaatctgggaaaaatgccctacaacgaagcaacaaaagttccaaccttatcgcaatggttcaacgatatggtaccgaagtatctttgctgtttgtggcaggaagaacaggcggtgggttgcgaaacgctgagattcgaaagaagaccaactcaggactgtgtcgcgtaccaagctccagggattgctgggatttacggagatccccacgtcatcactttcgatgacgtcgagtacaccttcaacgggaaaggagagtttgctcttgtgaaatctgtgacacaaactgacaacttggaggtgcaaggcagatttgagcaaatggaccctaacgcctacggagaagtacgtgcaacacaattgacttcaattgtggcaaggggaaacaacaccatagcagtggaggtcagaaggaggcccttggatgctaggtggaggtataggctggatgtcatagctgataataggaagttgttcttcgacagaccctctttgaaatttcaacatttccaaggagtgactatttatacacctacttatatcctcaatcagtctgaagtcatcattatgtttgataacggagcaggagttcaagtaatggataaccagggattcatgaccgcgagggtgtatcttccttggtcattcatcaacaaaactgttggtctctttggcaactggagtttcaataaggaagatgacttcactcttcctgatgagtcgaaggctgccgtcgtgagtaatatcaatgatatggaaagggtctacaatgactttggttccaaatggatggtggacgacgtactagatccgaaaagaggtagatccttatttttcagagaattcggcagatcatcggcaacgtacaacaacaaaactttcaaaccgcagttccttatgttacctgaggacataatacccgcaaacaggtcgatacagatacagagaacttacgacatttgtagcacaaaaatgtacgaatgcttctacgattatgccatgacgctcaacagagatcttgcccattttactcagaattataaagcaaccatatatcaactcaaagaaacgacgaggcagaaggttgtttcttgcggagttctggaaacaccgcgattcggtaggaagagtacttttctttttataccaggaaccaaagtcacttacgagtgcaatcaagacttcgtattggtgggagatcccagaagagaatgtttggcagatggcacatggaatgctcctgaatatggctacaccgaatgtttacgtcaacaagaatattctcagcgccaatcagccattgcctctggagccgttctcgcaataattattccactagttctattatttgtatatctggcttatatgttcctcaagaagaaacagaaagaacgagacgaagaaaatttacaaacgcaagcgtacgagcaacagaaaagacaagctcaggaagctgctgctagaaaattaactgctgccgaagagtacaactcagatgaagacgataataacagcaacgttacgagcacagcaaaagaaacaacagtgtattag

SEQ ID NO: 70 - DNA, 4455 bp, Helicoverpa zea
atgggtgttaaagttctagctttaatagcactcttagttattagtgttaatggacaagaaatagttgacaacgtcacaagtaatgttgctgaggacaatatagtaagtgatactacgacagtggtggtaccagtaactgaagtaaaggaagatgagattgataatgaaagtccagtggaaattataagtgacaaagcagaactgcaagttaggagtgggaagtaccagacattgactgatggactggggggcgaagagccgctggctttggatgcggttgatttaaacgtcaacgataatttgctcagtgaaaggcagttattgtcaccaagcaccactcaagttacgaacaatgagtatgctcacatcgacggccgtgtgctacccgacacgagctaccagaacaatggtcagccctacgtcatcacagctgccagactggcgcagatcagaggcaacttcatgtactggttctacgatcaaggaggcaacgagaatattggagactaccagagagacatccacacgtctactcctcaaatccataagaacttcaacttccagttgccgttctttggatttaggtttaattataccaggttatctatgaacggttacatctacttcagcgaccctccagaccactacacttaccccctctccttccccgtgagagactggcctgatgtcaacgacccctccttcattggtatattcttcagcaagtgccgtatcggtagtcagcgtcctgaggacccagatcagagaaggcctggaatttactttagaatggacagggatttgcagacacgtaccgaccaactgggtgtggagatgagagagcgtttgacgtgggacattcgtcagggtgtcatcggttccgagactttcttccccaagcacgccatcactatcacatggaaaaacatgtctttcgctggaggcattgacaactcgttgtttgtgacaaacacattccaaatggtactggcaactgacgaagtgttcacatacgcgatcttcaactatttggagatcaactggagttcacacacagaagctggtggtgataccactaccggagaaggtggtgtacctgcttatattggtttcaatgctggtaacggaacacaaagctacgaatacaagccttattcacaagcgtctgtacttagagatttgacgggcagaggatgggcgaacggttttccaggtcgacatatatttaggatagacgaaaaaatacttatggggacttgtaacaaggatatcgacggtgcaaatcttcctctcatgttcgcccctgagagcggtaacatgttgggaggtacaatcgtcaacatcacgggtccttgcttcaaccccaacgatagaatcacctgtcgtttcgatactgagtccgtcattggtgctgtagtggacgtcaatagggctatttgcgtccaacccaggttctggcacaatggatatgctagattcgaaattgcgattaataatgaaccctataagtggaaaggaaagtactttgtcgaaacacccgcaacggccacagagaagattttcttcactgacaactcagtccacgagagatatcctcctgaaatcagaataacttgggaccgcttcaacttgaccacgaatctaaacgtccaactccagatcagtttgtggggctacaaggaggtcactatcagacctcaactcgaatatattgacatgatcgaaatgggcgtggctaacaccggcgaatacgtcatcaatccacaaaactttaggaacagggataacttccttcacaatgacatgcagttcggtttcctacaaatcaacttgaccacaccagaagtgtttaaaaatgttgagatatcacctgtcctgtggagtcgtccaatccccttaggttggtacttcgccccacaatgggaaaggttacacggccaacgttggcccaacgccctttgtaacaactggttacgaacggatcgtttcttgaagaactttgcatctcaagtatgggtttgcccttgtacgctggaacatgcgttactggataagggaaggtttatgccagatttagcttgcgataaagacaccaatcccacctgtagataccattggggcagtgtccattgtgtcaggagtggagcacctagcgcggaaggatcaggtcagcagtgctgctacgacaagaacggcttcctcatgttgtcgtacgatcagatgtggggatccaggccttcgaggtctcacgacttcgggttcactccttacaacgaggccaacaaggtcccatcgctgtcccattggttccacgacatgattccattctaccagtgctgtatgtggcaagaagaacaagctgttggctgtgagacattcaggtttgaacgtcgtccttctcaagactgtgtagcttaccaatcgcctggtgttgctggaatatttggagacccacacattgttacattcgatgacttgcagtataccttcaacggaaaaggtgaatacgtattagtaagagtggatcacccgcaactaaaactggacgtccagggcagatttgaacaagttccacgtaacatctacggacgagtcaacgctacacatctgacttctgtcgtcgctgcttccaataactccgtacctattgaggttcgtctccgtccccaacatgctcaatggcgttatagacttgatgtattcgctgacaacaagagagtgtacttcgacaggcctgcccttagggtgcaatacttcccaggtgtgacagtataccagccaatgtacttactgaaccagtcggagattgtcatcatgttctcatcaggcgctggtattgaagtggtagagaataaaggctttatgtcagctagggtttatctgccttggaattatatgaaccaaacgcgaggtctattcggcaactggtctctggacattaacgacgacttcacgcgaccggacgggacgcgagccacagtcgacctcaacaacttccaaactgcacacagggacttcgctcaatactggcaactaaccgaccgcgaacaacgagacataggagtagcgatgttctaccgcgaatacggcagaacggcagcctactacaacgacaaccaattcataccaaacttcatcagggaaccagctgacttcctaccagcgaacaggtctcaagacgtggcgagagctgtggaactgtgccaggattcctatcaatgtcgatatgattacggcatgaccctcaacagggatatggctgagttcacgaagaattatttgtcttctatcacgaatataaaagagcagaacgctcgaagagtgatcagctgcggtatattggagacaccacgatttggacggaagagtaacttcttcttcacgccaggaacaagagtgaacttcgagtgtaaccaagacttcattctgattggagacaagcgacgagtgtgtgaggacaacggccggtggaacttaccggattatggatatactgagtgcttgcgtaaccaagaatactcgcaacgttctctgtttctgacctgggcgatcatactggcagttattctaccgttaggcctgttgatttgtctgctgtggttctggtgctggtacaaacccaggaccgaaggcaaagaaggattccgtttcgaagatattccgcgatcaaaatcagcctctagacttaatctaagatcagcatcaatgggaaacctcacggatacgatgaaatcatccacattacgaagctccgactctaagcctaagttaccagaaactcctactgaggaagccccaatgactaggtctgcacctctagttaggccagctccacccgtcccacaagacggcgatagttcaggaatagggtacccagattccaataagagtgacaaatctgacaaatcaaatcctaaaaaacgtagggcctatgacaaaacttaccgaaccaatgaacctatcccgaacgctcccaatgaagaattcccagaaaaactgtgggatctttccgaagaggacttgctttctataacttcaccgtcagacactgaatctaatagagattcaacgttgacgcgacctgctaaagacattgaatatcaaagcagacctcgccagacaggtcgtagagcactgccaagtgactcgggttattccacaaaagattccgaggacccatactctccgaagtatgaaggtcagtacagtcctattccttcccagtactctccaacgtactccgaaatatattctccaccgatcagccctacttcggacaatagtccaagaaacacgttcaataaccccggtttgcctgaccctccgaaaagcgctcctgcagattctattcagacgtttaccatgcctccccaaaaggggaaatctttggagactctgattgacccaccgaacattgaagtgcccacaatgagccctcgctcgaccatggtctga

SEQ ID NO: 71 - DNA, 4473 bp, Spodoptera frugiperda
atgggtgttaaggttttagctttaatagcgctcttagttgttagtgtactcggacaagacgttactgtcagtgacaacgatgtaacaaaggaagttgtggtggacaatttacctagtgagaacccgatagtattagacacagttacagaagccacaaaagatgaagttgctgaagatgcgaacccagtggaaatactaagtccaacggatgatctgcaagtaagaagcgggaaatatcagctcaatgatgggctcgtgggtgaagagccgatgccactagatgctgttaatttcgactcgaataatgatgccggagagagtgagaagcagttgctctctcctggcacaactcaagtcacgaacaacgagtatgctcatatcgatggccgagttttaccagacaccagctatcagaacaatggccaaccctacgtcatcacggctgctcgactggcccagatcagaggaaacttcatgtactggttctatgatatgggaggtaacgagaacaatggagattaccagagggacatccacacttccactccacagatccataagaacttcaatttccagttgccgttcttcggatttaggttcaattacaccaggttgacgatgaacggctacatctacttcagtgaccctccagaccactacacataccctctctctttccccatgagagactggcccaacgtgaacgatccgtccttcatcggtatcttcttcagcaagtgtcgtatcggtagcctgaaccaggacgaccctgatcaacgaagaccaggagtttactttagaatggacagagatctgcagactcgtacagaccaactgggagtggagatgagagagcgtctgacatgggacatccgcgagggtgtgatcggttccgagaccttcttgcccaaacacgccatcaccatcacctggaagaacatgtccttctctggaggaatcgacaactctctgtttaggacaaacacattccaaatggtgctagcgactgatgaggtgttcacgtacgcgattttcaactatttggagatcaactggagttcgcacacggaggctggaggtgataccactacaggagaaggtggaacgcctgcttatatcggattcaacgctggtaacggaacccaaagctatgaatacaaaccctactcacaagcatctgtgcttcgagatctgacgggcagaggttgggccaacggtttccctggacgacacatattcaggatagacgagaaaatacttatgggaacttgtaacaaggatatcgatggggcaaatcttccgttgatgttcgctcctgagagcggtaacatgttgggaggtactatcgtcaatatcacgggtccctgcttcaaccctacggacagaatcacttgccgtttcgacacggagtccgttataggtgccgtagtggacgccaacagggctatatgcgtccaacccagattctggcacaacggatacgccagatttgagatcgccatcaataacgaaccttataaatggaagggaaaatacttcgtcgaaaccccagcaacggctgcagaaaagatattctttactgacaactcagtccatgagaggtatcctccagagataagaataacttgggaccgcttcaacttgacttcgaacctcaacgtgcaacttcagatcagtttatggggctacaaagagattacaatcagaccccagcttgaatacatagatattatcgaaatgggtgttgctaacacgggagaatatgtgatcaatccccaaaactttaggaacagggacaacttcatgcacaatgacatgcagttcggtttcctccagattaatttaactacgcctgaagtcttcaaaggagtctcaatctcgcctgttctttggagtcgaccaattccattgggttggtacttcgccccacaatgggaaagactgcacggctcacgttggccgaatgccctttgcaacaactggctacgaacggatcgtttcttgaagaacttcgcttctcaagtctgggtctgtccatgcacattggaacacgccttattggacaaaggaaggtttatgcctgatctggattgtgacaaagacacgaaccccacttgcagataccactggggaggtatacactgcgtcaggagtggagcacccagtgcggaaggttctggccagcagtgctgctacgacaagaacggtttcctcatgttgtcctacgatcagatgtggggatccagaccctcaagggctcacgacttcggattcactccttacaacgaggccaacaaggtcccatcgttatcccattggttccacgatatgataccattctaccaatgttgtttgtggcaagaagaacaggcagttggctgtgaaacattcagattcgaacgtcgaccttcgcaagactgcgtagcataccaatctccaggagtagcaggaatattcggagacccacacatcgtcacattcgatgatttgcaatacactttcaatggaaaaggtgaatacgtcctggtacgagtggaccacccacaactgaagctggacgtccagggtaggttcgaacaggttcctcgcaacatccacggcccggtcaacgccacacatctcacatctgttgtagctgcttccaacaactccgtacctatcgaggtacgtctacgcccccaacacgctcaatggagataccgactcgatgtgttcgcagacaacaagagggtctacttcgacaggcctgctctccgagttcaatacttcccaggtgtgacagtgtaccagccgatgtacttgctgaaccaatcagagattgtgatcatgttctcgtcgggcgccggtgtcgaggtgatcgagaacaaagggttcatgtctgctagagtctacctgccttggacttatatgaaccaaactcgtggtctattcggcaactggtctctagacgtaaacgatgacttcacgcgacctgatggcacgcgagtcgccgtggacctcaacaacttccagactgcacacagggacttcgctcagcactggcaactaaccgacagagaacaacgagacataggagtggcgatgttctaccgagaatatggtagaaccgcagcatactataacgacaaccaattcgttccgaacttcataagggagccggcagacttcctccctgctaatagatcgcaggacgtggccagggcgatagaaatctgccaagactcctaccaatgccgatacgactacggtatgacactcaacagagacatggctgagttcaccaagaattatttgtcttctattacgaacattaaagagcagaacgcccgtcgagtcatcagctgcggtatattagaaactcctcgattcggaagaaagagtaacttcttcttcacaccaggcacaagggtgaacttcgaatgtaaccaagacttcattctgattggagacaagcgtcgagtgtgtgaagacaacggacggtggaaccttccagactatggatacaccgagtgtctacgtaaccaagagtactctcagcgcgcactgttcttgacctggggcgtcatattggcagttattctaccgttaggcctgttgatttgcctactgtggttctggtgctggtacaagccccgctccgaaggcaaagacggattccgattcgaagatattccgcgatcaaaatctgcttccagactaaacctaagaacagcctcgatgggaaatcttacagacacaatgaaatcttccactttacgtagcacagattctgataaaaaggctaaattgcccgaaactcccactgaggagaccccaatgactagatctgctccattaactagggccgccccacctcccccacaagatggtgacagttcaggcatagggtacccagattccaataagagtgactccaataaatctgacaagtcgtttaaaaagcgtagggcctatgacaaatcctatcgaaccaacgaacctatcccgaacgctcctaacgaggaattccctgaaaaactttgggatctatctgaagaagacttactttccataacatcgccttcagataccgaatcaaacagagattcaactttaactcgtccggcaaaagatatagaatacgttggcagaccacgtcagttaggtcgcagggcgttgcctagtgactcaggttattctacaaaggattctgaagatccttactctccgaaatacgagggtcaatacagccccataccttctgcgtattctccaacttattccgagatatattccccaccgatcagtcctacctcagacagtccgaggagtacattcaataaccctggcttgccagaccctccaaagagtgctcctgcagattctatacagacattcaccatgcccgcacaaaagggcaagtccttagagactctgatagacccacctatttccgaaatgcccacaatgagccctcgctctaccatggtctga

SEQ ID NO: 72 - DNA, 4494 bp, Ostrinia nubilalis
atgggtgttaaagttttagttttagccgcgtttctagttataagtgcaagtgtatacgctcaggacgttgtagacaatgtgacagacagaactgttgatgaattattattagatgtggacccaaaattagaagttacaccgccgacggaagaattgccgcagccaaatgatgaagtgcctgaagaatcggcagttgaagttatagcagatgttccggcagctgatttggagatccgaagtgggaagtaccagctgaacgatggcttggtgggtgaggagccagtggaactggaagcggtggccaatgtcgaaccggacccaaatgctgagcagagtgagaggcaactcctatactcacctgactctacaatgaacaccaattataataacgaatacgcacgcatcgacggaagaatactccctgatataacctaccagaacaatgggcaggcttacatcatcaccgcccagcgcctccagcagatcagggctaacttcctctactggttctacgaccagggcggcagcgagaacatcggagactaccagagggatatccacacatccaccccgcagatccataagaacttcaactttcagctgccgttctttgggttcagattcaattacacgaggctctccatgaatgggtacatctacttcagcgatcccccagaccactacacctaccctctctccttccccatgcgggactggccgaaagtcaacgatccttcttttatcggtattttcttcagcaagtgccgtatcggcagcatcagacccaccgacattgaccagagaagagctggcgtttacttcagattggacagggacttacagactcgtagagaccagcttggcgtagaaatgcgcgaacgcatcacctgggacatccgtgaaggtgtcattggcgctgagaacttcttcccgaaacatgctatcacgataacttggaagaacatgtctttcgccggaggaattgacaactctctctttgtgactaacacgttccaaatggtactggcgacagatgaagtgttcacatacgcgatctttaactatcttgaaatcaactggagttctcacacagaagctggtggtgataccaccacaggagaaggaggagtgccggcttatattggtttcaacgccggtaacgccacccgaagctatgaatacaaaccatactctcaagagtctgtactccgtgatttgactggaagaggatgggctaatggtttccctggtcgacatatatttagaatagacgaaaatatactcatgggaacatgtaacaaagacattgacggtgcaaatcttcctctgatgttcgcccccgaaagtggtaacatgttgggaggtacagtggtgaacatcactggcccttgcttcaaccccaacgaccgaatcacttgccgtttcgatactgaagccgtggtgggtgtcgtcgtggacgtcaatcgagccatctgtgtacaacctcggttttaccacaatggttatgccagatttgaagtcgccatcaacaacgagcctttcaaatggaagggacgcttctttgtagaaacccctgcaacagcaaccgaacgaatctacttccctgacaacgctatccaccagaggaatccaccggaaatcaaaataacatggaaccgtttcaatttgacgaccaatttgaacgtccagcttcaaatcagcttgtggggctacaaagaagtcaccatcagaccccaaatggaattcatcgatatcatagaaatgggtgtggcaaatactggggagtacatcatcaacccacagaacttcaggaatagggataatctgatgcacaacgacatgcaattcggttttctgcaaatcaatttgacaactcctgagatatacaaaggcgttcaagtttcaccaattctctggagtcgaccaatccctctagcctggtacttcggccctcaatgggagcgtctccacggaacccgctggcctcaaaccatgtgcaacaactggctccgaaacgatcgtttcctcaagaactttgctgctcaaatctgggtgtgcccctgtactctggaacatgctttgctggacaaaggtcgtttcctaccagatctcgattgtgacaaagacaccaacccaacttgtaggtatcactggggaggtgtacattgcgtcaggagtggagcgccaagtgctgaaggatcaggtcaacagtgctgctacgataagaacggctttctcatgctttcatacgatcaaatgtggggatctagacctcacaggtcacacgactttggatttacgccttacaacgaagctaacaaggtaccatcgctgtcccattggttccacgatatgattccattctaccaatgttgttcatggcaagaagaacaagctgttgggtgtgagactttcagattcgaacgtcgtccatcccaagactgtgtcgcgtaccaatctccaggagttgcaggcatattcggagaccctcacatcgtcactttcgatgacctccagtacacatttaacggcaaaggtgaatacgtattggtgagaacagacgaccctcaattgaaactggacgtgcaaggtagattcgagcaggtcgcgagaaacattcatggcgtagtcaatgcaacccatcttacttctatcgtagctgcatctaacaactctgttcctattgaagtcaggttacggcctcagcatgcgcaatggcgataccgactggatgtgttcgctgacaataaacgtgtctactttgacaggtctgctttgaggattcagtacttcccaggtgtcactgtataccagcctatgtacatcctcaaccagtcagaaatcgtgatcatgtttgcatcaggcgctggagttgaagtggtggagaacaagggcttcatgactgccagggtttacctaccctggacgtatatgaacaaaactcgaggtttattcggtaactggtcgctagatgtaaacgatgacttcacgcgccccgacggaatagtgcaacccattgatctcaacaacttccaatcggcacacagagatttcgcgcaacactggcaactaactgatcgggagcaacgaaatataggcgtagcgttattcacaagagaatacggtcgaactgcggcgtactttaacgacaacaccttcataccgaacttcatacgagagccggcggacttcttgcctactaaccggtcgcaggatgtggcaagagcgatagacatctgccaggactcgtaccagtgtcgatacgacttcggcatgaccctgaatagggacatggcggagtttacaaagaattatctgtcttcgattacgaatataaaagaaacgaatgcacgaagagtgatcagttgcggcattttggaaactcctcggtttgggcgaaagagtaacttcttcttcactcctggtactcgggtgaacttcgagtgtaaccaggacttcattctggtcggagacaagcgcagggtgtgcgaggacaacggccggtggaacctccccgactatgggtacactgagtgcttacgtaaccaagaattctctcagcgcgccctatttctgacgtggggtgtgatagtggcaatcattctacctttggccctcttgctctgcctctcgtggttctggtgctggcacaagccgcgctccgagggcaaggagggattcagcttccaggactacccgcgatccaagtccgcttcacggctcaaccttcgatcagcttcaatgggaaatataaccgacactgcgaaatcctccactttacgcagtcaagacacagacaccaaacccaaattacccgaaactcccacggaggaaactcccatgaaaccaagcatgcggtccgctccgctaccccctgaacctcaggatggtgacagctcagggcttgggtatgccgactctagtaaaagcgactccggcaaatcagacaagtcttccattccgaaaaaacgtcgctacgataaaacttaccgaaccaacgaacccttaccaaatttacctgatgtagagttccctgaaaagctctgggatctctctgaagaggacctgctttcgcttacctctccatccgaatctgagtctaatcgtgactctactttgacccgtccggccaaagacattgaatacgtgagcaaacctcgccagactgggcgtcatgctcttgccagtgattcgggctactccacaaaagatggttctgaagatccctatggtaacaaatatgggggcacttacagtcccataccttctccaacttattctgagatttactctcctgccgtgagtccgacttctgacatcagtccgaggagcacattcaataacccgggtctaccagaggcccccaaaagtgcccctgctgatgagataaaaacgttcactcttcctccagtccgtggaaaatcagtagagactttaatagatcctccagttgctgaaatgccgaccttcagcagtcgctctacaatggtttaa

SEQ ID NO: 73 - DNA, 3807 bp, Phyllotreta cruciferae
atgaactttatactagttttaataatcagtgcggtggtcgtgggggctcaagagggcgtcgcgccggacgccgcgggccaggataacacgggagacgtggagagcatcgacgtgggtgcgtcaggaggcgacgaaggggtcgattccaagcccattcccgtcgaagaaaacggcgggagtgttactactaacattaattacgacgctcccgtagaagtattaacaaatatcgatagcgaaccaattgatactaacaattccatagccaactggacttaccagaaaaaaactaaatccgggaaatatgttacttattatgcggccaattatgaccccatgacatcagatacagccccccaggacacggaccaaaggggtggaggcttgccttactccattaccgaaactaggttagccgagattaggaaaaactttatgtatccgttcttcgacaaagggggcaacgtggataacgagggggactatcagaaggaaatccaatcctccacgccccaggtccacaagaatctgaacttccaattgcctttcttcggattcaggtacaattacacgagagtgtccattaatggttacttggaattcagcgatccacctcccaacttggattacccattagtttttcctgtaaaggactggcctaaggagaatgatccttcgtttattggtattttctacagtaaatgcaaaatcggtaatcttcgcgacgaagactcggacaaaaggaaaccaggtgtgtacttcagaatggaaagagacctcagggacaggcaagacaggatgggagtggaaataagggaaaggttgaaatgggacataagagaaggagttatcggttcggagaccttcaatcccaaacacgccataatcgtaacgtggaagaacgtatccttcaatggaggatacgccaacgcagtttatcagaccaacacattccaaatggtcctcgccactgacgaggtattcacctacgccatgttcaactacctaaatttggactggaccagtcacaccgaagccggaggagacactagaaaaggcgaaggcggtacgccggcattcgttggattcaacgccggtaacggcaccagaagcttccagtacgctccctacagtcaagcatcggtcattcgggacttaacatccaccggctggggtaacaacaaacccggcaggcacattttccgaatcgacgagaacatcctgctgggttcttgtaacaaagatatcgatggagctaacttgcctcttcaatttgctccggagagcggcaatatgctgggaggaacggtcgtgaacatcacaggtccttgcttccaacccaacgacaggatcagatgtaaattcgacgtgtacgatgaagttgttggattcgtggtggacaggaatcgtgctatttgcatccaacctcgcctttactccgagggttgggtgaggctgcaaatcgccatcaattcggacgccttcaaatggaagggaaaatactacgtcgaatcgccggccagtgcctcgcagaagatcttcttcaaagacatgaagtaccacgagaagtcgcccagcgagattcgcatcacgtggcagaagcagaacttgaccaccaacgaaaatgccaacatcagaatttccttgtggggttacagggaaaccaccatgaggcccacgttcgtttacatcacagaaatagccgataatgtccaaaacaccggggagtacacgatcgtaccgtcgcagtacagaggcaaaatcaaccagttcctcaccgacattaagtttggattcctccaaataaatttgaccgaatcgataaaggtcaacatttactctaccaacaaccaaaacatcgacatcacaccgataatatggagcaggccgattcctctaggttggtatttccaattccaatgggagaaccaatacggcaaaaattggcccaaacaactctgcgacgattggctcagaaccgatagatatctgaaaaacttcgctcacgaattaccccaatgtccttgcacggtccaacaggctctggctgataaggggaaattcatgccagactttgattgcgacaaagattcgaatcccgtttgttattacaacaaccaagctttgcattgcgtcaaaaccggtgctcccacaatggaaggctccgagcaacaatgttgctacgacaaaaacggctacctaatgctgtcctacgatcaacaatggggttccagcccccgtcgatgccacaaccttggaaaaatgccttacaacgaagccaccaaggtccccacactatcccaatggttcaacgacatcgtacccaagtacctctgctgtctttggcaggaagagcaagccgtgggctgcgaaacgctgcgattcgaaagaaggccttcgcaggactgcgtcgcctatcaagcgccggctgtggcaggagtgtacggtgatccgcacattgtcaccttcgatgacgtagagtacacgttcaacggcaaaggagagtacgccttggtgaaatccttgaaggtggacgataatttggaaatacaggctagattcgagcaaatggacatcaatgcttatggagaggtcagagccacgcaacttacggcaatcgtagccaaaggaaacaagaccattcctattgaagtgagaagaagaccgttggaagccaggtggaggtatagattagatgttatagccgagaacaggagattgttctttgataggccttcgttgaaattccaacatttccaaggggtcactgtttatacaccaacctacattctgaatcaatctgaagtactaataatgttcgacaatggagccggcgtcgaagtgctggacaatcaagggtacatgagcgccagggtgtacttaccttggtcgtttattaacaaaacggtagggctgttcggcaattggagcttcaacaaagaagacgacttcacgctgccggacggatccaaggcggccgtggtgagcaacatcaacgacatggagagagtgtacaacgatttcggttcaaaatggatgctggacgacgtaggcgacgcgcacaaaggccagtcgctgttctacagggaattcggccgcacgtcggccacctacaacaacaagaccttcaagccgcagttcctgatgctgcccgaggacatcctgccgcccaacaggtccgagcaaatacagcgcacgtacgacgtgtgctcgaccaagatgtacgagtgctactacgactacgccatgacgctgaacagggacctggcgcacttcacgcagaactacaaggctaccatttacgagctgaagcaaaccaccagggagaagatcacgtcctgcggcgtgctggaaacgccgcggttcggcaggaagagtacgttcctgttcgtgcccggcacgaaggtgacctacgaatgcatacaagatttcgtgctcgtcggtgatcctagaagagagtgcgggggtgatggaaaatggaacataccggattatggatacacatattgtttgcgtcagcaggaatattcccaacgacaagcaggaatcacgtccgggataataatgttagtgctcataccactcatcctgctattcgtgtatttggcgtacaggttcttaaagaagaaacagaaagaacgggaggagaacaggctggagcaagaacaatacagagaacaatctaggaaagcgcaggaggcggcgacgatgaaattgaccgaacacgaggaagagttcaattctgaggaggaagataatcatagtaacgtgaccagcacgggagctaaagaaacaaatatttattag

SEQ ID NO: 74 - DNA, 3837 bp, Phyllotreta striolata
atgcatgtgaaaatgaactttctgctggtgttaataatcagtgcggtggccgtcggggctcaagaggatgccgtagcgcccgatgcagcgagccaggataatacggaagacgtggaaagcatcgacgtgggtgcagcaagaagcgacgtcggcgacgccgatcccaagcccgttcccgtcgaggaaaacaacggcggggcggttacgaataacctcaattacgacgctcccgtagaacaacgctcccgtaaattaacaaatttcgatagcgaaataattgatactaacagttccaaacccaattggactttccagaaaaaaactaaatctggaaaatacgttacttattatggggccaattatgaccccatgacatcagccaccgcccctccggacaccgatcaaagaggtggaggcatcccttacctcattaccgaaaccaggttagcggacattaggaaacactttatgtatccgttcttcgataaagggggcaacggggacaacgagggggattatcagaaggaaatccaatcctctacgccccaagtccacaagaatatgaacttccaattgcctttcttcggattcaggtataattacacgagagtctccatcaacggttatttggaattcagcgatccacctccgaattacgattacccattagtttttccggttaaagactggcctaaacgaaacgatccagcttttattggtattttcttcagtaaatgcaaaatcggtaaccttcgcgacgaagacgccgacaaacgtaaaccaggtgtatacttccgaatggaaagagacctcagggacagacaagacagaatgggagtggaaataagggaaagattgaaatgggacatcagagagggagtaatcggttcggaaaccttcaatcccaaacacgccataatcgtcacatggaaaaacgtatcgttcaatggaggattcggccccgcagtttttcaaaccaatacattccaaatggtcctcgctactgacgaagtattcacgtacgcaatattcaactacctaaacttggaatggaccagtcacacggaagccggtggagacaccagaaacggggaaggcggtgtaccggcattcgttggattcaacgccggtaacggtacaagaagctaccagtacatgccctacagtcaagcatccgtcattcgagatttgacagcgaccggttggggtaacggcaaaccgggaagacacattttccgaatcgacgagaacatcctgctaggttcttgtaacaaagatatcgatggggctaatttaccccttcaatttgcgccggagagcggtaatatgttgggaggaaccgtcgtcaacatcacaggtccctgtttcaatcccaacgataggatcaggtgtaaattcgacgtgttcgatgaagttgtcggatacgtcgtcgacaggaacagagccatttgcattcaacctcctctttacacagaaggttgggtcagactgcaaatcgccgtcaattcggaagcgttcaaatggaagggaaaatactacgtcgaatcgcccgccaccgcctcccagaagatcttcttcaaagacatgaagtaccacgagaagtcgcccagcgagtttcgcatcacgtggcagaagcagaatttgaccaccaacgaaaatgctaacatcagaatctccttgtggggctacagggaaactaccatgagacctacgttcgtttacataactgaaatagccagtacccagaacgccggggagttcctgatctcgccgtcgcagtacaggggcaaagtcaaccaattcttaagtgacatcaagtttggattcttgcaaataaatttgactgaatcggtaaaggtcaacatttactcgacgaccaatcaaaacgtcgacattacaccggttatttggagccggccgattccgcttgcttggtatttccaattccaatgggagagtcaatacgggaagaactggcccaaacacctctgcgacgattggctcagaacggacagatatctgaagaatttcgcacacgaattaccccaatgcccttgcacagtccaacaggctttggcggacaagggaaaattcatgcccgactttgattgcgacaaagattcgaatcctgtttgttattacaataaccaagctttgcattgcgtcaaaactggtgcacccacaatggaaggttccgagcaacaatgttgctacgacaaaaacggttacctgatgctatcctacgatcaacaatggggttcgagtcctcgtcgatgccacaacttgggaaaaatgccttacaatgaagccaccaaagtccccacgctgtctcaatggttcaacgacatagtgcccaaatacctctgttgtctctggcaggaagagcaagccgtgggctgcgaaaccctgcgattcgaacgaaggccttcgcaggattgcgtcgcctatcaagcgccaggtgtagcaggagtgtacggtgacccgcacatagtcaccttcgacgatgtcgaatacacattcaacggtaaaggggagtactccttagtgaaatcggtcaaggtggacgataatttggaaatacaggcgagattcgagcaaatgagccccaatgcttatggagaggtgagagcgacgcaactaacggcgatcgtggctaaaggaaacaagactattcccattgaagttagaagtagaccgatggaatccagatggaggtatagattagatgttatagccgagaacaggaggttgtactttgatagaccttcgttgaaattccaacatttccaaggcgtgactgtttatacaccaacctacattctaaatcaatctgaagtactgataatgttcgacaacggcgccggtgttgaagttctggacaatcaagggtacatgagcgccagagtatacttaccttggtcgtttataaataaaacggttggattgttcggaaattggagtttcaacaaagaagacgacttcaccctgccggacggttccaaggcggccgtcgtgtcaaacatcaacgacatggagagagtgtacaacgacttcggttcgaaatggatgctcgacgacgtcggcgacgagcgcaaaggccagtccttgttctacagggaattcggccgcacctcggccacctacaacaacaagaccttcaggccgcagttcctgatgctgcccgaagacatcctaccgccgaacagatcggagcaaatacagcgcacctacgaggtgtgctccacgaagatgtacgaatgttactacgactacgccatgacgctcaacagagatttagcgcactacacgcaaaactacaaggcgaccatttacgaactgaagcaaaccaccagggagaagatcacgtcctgcggcgtgctggaaacgcccaggttcggcaggaagagcactttcttattcgtacccggcacgaaggtgacttacgaatgtatacaagatttcgtgctagtcggtgatcctagaagagagtgcggggctgatggaaaatggaatattccagaatatggatacacgtattgtttacgtcagcaggaatattcccaacgacaagcaggaatcacgtccgggataataatgttagtgctcataccgctcatcctgctgttcgtgtatttggcgtacaggttcttgaagaagaaacagaaagaacgggatgaaagcaggatgcagcaggaccaatacagagagcaatctaggaaagctcaagaggcagcgacgaggaaattaaccgagcacgaggaggaatacaattctgaagaggaagataataacagtaacgtgaccagtacgggagctaaagaaacaaatatttactag

SEQ ID NO: 75 - DNA, 3429 bp, Megacopta cribraria
atgcaggtggctgtgctgcttttgtgcttttgtttcctctcgagcgccaaagaaccgctcagagtcagtgagaacttgttaaatgcttacagatacaggaactatggcagagtcttgggatatggcaacaatactcgtttttatgacgacgaggacgaaaggatggttccttcagacgaccgtaataacggaaattatgtgttgaccgaagacagattgaggaaaataaggtcaaaatttttgtattggtttttcgataaagatcctaaaggaggaactggagatttacaaagagacattactccttcgatcacacaaatccataaaaatcttaatttccaactacctttttttggatatcgctttaattatactagagtgtctttgaatggctatttggaatttagcgacccaccagagcaatattcttatccgctaagttttccagttgtggattggccacataaaaacgacccttcgttcattggaatattctacagtaagtgtagagtgggacaaatgggggaaaatgatgttgatcagagagagcctggcgtttattttagaatggagagagatttaatgtcaagaacagacaaatttggcgtggaggtgagagaaaggttaatgtgggacattagagaaagcgtcgtcggttcggacaccttcattccaaaacatgctgtaattgtcacgtggaagaatgtttcatttgctggtggtattgagaactcagtgagaacgacaaataccttccaattagtcctagcaacggatgaagtatttacctatgcaatgttcaactacgcacaaatcaaatggacaacgcacactgaagccagaggagacacagttggaggagaaggtggtgtaccagcttacattggattcaatgctgggaatggtactcgaagctacgaatacagaccttactcacagcaatctaccatcagagatttagttggccgaggctgggctaatggttttcctggtagacatatttttagaattgatgaaaatattcttcctgggacttgtaacaaggatattgctggggctagtttaccacttacatttgcaccagaaagtggaaacatgctgggtgggacaatcgtcaacataaccggtccctgctttaccccacaaatgaagattatttgcaggttcgatatggaagcagtttatggtacagtcattgatactaatagagcaatatgtgtgcagccttacgttatggcggaaggatacatactttttgatattgccgttgatgatggaaaatatgtgtggaaaggacaatattttattgaaacgccggctacagctgcagaaaaaatatcctttgcagatgacaatgttcatcaaaaatacccaccagaaatcagaataacttggaatcagtacaatttaacaacaaatttagcagctccagtccaaatatctatctggggatacagagaaaaatcaatacgtccacaattaacttacatagatttaatagacgctggagttccaaataatggaatgtatacaataattccatcatcttttaagctacgtgataaccgtatgttcacagacataaattttggatttatacaaattaatttaacaaatcccactaattataatgggctcaaagtaaccccggttatctggagcaaacccatacctttaggttggtattttgcaccacaatgggaaagacaatatggcgaaaattggcctgagcaaaaatgtaacatttggacaatgaatgatcgatatttgaagaattttcctgcagaattgagtatgtgcccgtgcaagttggaacatgctttggcagacaaaggaaggtttatgccggattttgattgtgacatggatgccaatcctcactgcttctatcataaaggggcaaggcattgtgttaaaactggatctcctaggcaagaaggctctgaacaacaatgctgctacgataagaataattatttaatgctttcctatgatcagcagtggggatcgtcaccaaaaaggtctagtaacttgggaattctaccatggaatgaaccaaacaaggtcccaaccctgtctcaatggtaccacgatatagcacctttctatcactgttgtctatggcagtatgaacaagcagtaggatgtgaaacgttcagatttgaaaggagaccctctcaagattgtgtgggttaccaatcaccctacgttgcgaccgttttcggagacccacactttattacttttgatggtttagagtatacattcaatggcaaaggtgagtttgtattactgcacaccagtactgacaagttcaaattagatgtacaagcgcgtttcgagcaagtggggaaaaatatttatggtgatgttgcagccacccaactgacatccctagcagctagagacaatacatcaagtatcatagaagtaagaatgaggccaaaagaggcccaatggagatacagacttgatgtctttgcaaacaagagaagaatatattttgatagaccttcactccgtgtacaacattttacaggtgtcactgtttatcagccatcatatatactcaatcaatcggaaatagtcatcatgttccaatctggagtaggtgttgaggtagttgaaaataaagggtacatggctgcaagagtttatttgccttgggaatacattaatagaactcggggactcttcggtaattggagtttcgatccaagtaacgatttcactcttccagatggaactgtaatgcccgtagaaaaccttaatgattttgaaagaatacataaggacttcgctattcattggaggctggaagataacgttgaagaaagtaaaggtgagcctctttttgttagagagtatgggagaacatcaagctattatttgaatagaacttttactccagtttggaaacgtacgcctcaagaaataattcctccaaataggactaaagatatacaaactgctttgagtttgtgtggagaatcttaccagtgtcgatatgattatgctgtcacgttaaacagggagatggcattttacacattaaactacttcagtgaatttactcatattagacaaacaaataaagaaagagttctgtcatgtggagtgttagaaacaccaagatttggtcacaaatcaaatttcttctttgtacctgattcaaaggtaatatttgagtgcgatcaaggatttatcctagtcggagatcaacgacgaacatgttcacctcaaggaagatggtcatatgaatatggttacactgaatgtcttcggtatattgaatatgatgctcagcagcttggaagaacgatcctaataattggaggagtattggtgcctttgataatgattataatttgcgcagtcgtataccaaaaggggacagttcacaagtttcctggaagttttcaaaagtag

SEQ ID NO: 76 - DNA, 4287 bp, Tribolium castaneum
atgttgacaaagtgtgtgttggtgattcttctactaagtgcatacaaccaagctcaagaaataaacaccagcgatgtagaaattttatcaaaaaatgatccacaaccaccgctggttaatcaagtggacgaaaatccgaatcagggtgggaacgacgacagaccggatcaaccggaaaacgaaagagaaaaatcagtccctaataacaccaccaaattagatacttctgacgatgcggttgaagtttttgaatcgccagatgtagttttgcgcaaagggcctaacggaaaatatatgtttgattatggcgacaattacgacccaatgacctcgcgctacgcccctcaggaagacagtcagagaggtcgcgccgggcccccttacgtcatcacggaagcccgccttaaagaaatccgggaacacttcatgtacccttactatgaccgcggtggcaatgctgataacgaaggagattatcaaaaagccatccagacatcaaccccccaagtacacaaaaatctcaacttccagttgcccttcttcggctttagatacaactacactcgagtctctctaaacggttatctggaattcagtgacccgccccaaaattacgaaacctatcctttggtattcccggtgaaagactggccgaaaaagaacgatccggctttcattgggattttttacagcaaatgccggattggcaatttgcgagttgaggatatcgaccagagaatgcccggcgtttacttccggcttgaacgggatttgagggaacggaacgaccagatgggcgttgagattagggaacgcttgaagtgggatatccgagagggtgttattggggctgataacttcaaccctaaacacgccattattgccacttggaagaacgtttcttttgctggaggattcgccaacgcgctgtacaaaaccaacaccttccagctggttttggccactgatgaagtctacacctatgccatgtttaattacttgaaccttgactggaccagtcatactgaagccggtggtgacaccatcaatggtgagggcggtattcccgcatatgtgggctttaacgcaggaaacggtactcgatcgtaccaatacgtgccatacagtcaaaattcggtcattcgagacctgactgggaccggtttcggtaatgaaaaaaaagggcgtcactatttccgaatcgacgaaaacatttacctcggaagttgcaataaggacatcgacggtgctaacttagacctgtttttcgcaccagaaagcgggaacatgctcggtggcaccattgttaacatcacaggcccgtgttttaaaccaaccgaccagattatttgcaaatttgacactgctgacgaagtttacggcgttgtggtcagcagcaatcgggcgatttgcatccaaccgccacttttggtcgaaggatatgtcaagcttgagatcgcgattgggcccggaacatacaaatggaaggggaaatattatgttgaaaccccggcaactgccactcaaaaaatcttcttcgacgacatgagctacaacgagaaatcgccggccgagattgggttcacttgggaaaagcagaatttaacaacgaacgaaaacgccaacatccgaatttcgctttgggggtaccgggaaacgagcgtccgtcctgacttcctctacattaccgatttggccgagaatgtgcaaaacacggggtcgtacaagattatcccgtcgacgtacaagaaccgcaacaatgagaaattgattgatatacaatttggtttcattcaaatcaacttgactgagtccattccggtgaccaacaagaacggacaaggcgaggtcaagataacccctctgatatggagccgcccgatacctttgggctggtacttcgggccgcagtgggagcgcaaatacggcacaaactggcccaagtatctctgcgataattggctcaaagacgaccgatatttgaagaattttgccagcgaactgccgcagtgtccgtgcactttgcaacaggccttagcggacaaggggcgctttatgccagattttgactgcgacaaagacgccaatcccaagtgctacttccaccgacaagccgtgcactgcgtcaaaacgggctctccctcgttgcaaggggcggaacagcaatgctgctacgacaaaaaccactacctgatgttatcctatgaccagaaatggggctcctacccgcgccgtagccacaaccttggctacctcccttggtacgaatcgaccaaagtcccgactttatcccaatggtacaacgacatgatccctcgcttcgtctgctgcttgtggcaagaggaaaccgccgttggttgtgaaaccctccgatttgaaagacgtccaacccaagattgcgtagcttaccaagccccggctgttgccggtgtttacggcgacccccacatcctcacattcgacgatctcccttacaccttcaacggaaaaggcgaatttgttttggtcaagtcggaaagtgtgcgaaatcgcttggaaatccaagccaggttcgagcaaatgcccatgaatgcctatggcgaagtaagggccacgcagttgacttcggtggtggctagaggcaatagtaccacgattattgagatcaggagacggccacaagaggctaggtggaggtatcggcttgatgtgatcgccgataggaggaggatttacttcgataggccttcactcaaattccaacacttccccggagttacagtttacaccccgacttatattttcaaccagtcggaagttatcgtgatgtttgattcaggggcgggtcttgaagttgtcgaaaataacgggtatttgacggcgcgggtttacctcccgtggtcatttatcaaccaaacgcgcggtcttttcggcaattggagcttcgatatcatggacgattttacgctccctaacggtgacaagaaaggggttgtgactaatttgaacgacatgtcgagcgtttacaccgactttggtatgaaatggatgttggatgatgtgggagatgaccagaaaggcgccgctcttttcttccgagagcacggaaaaactgcctcatcttacaacaacaaaacgttcgtccccgagttcagaatgtccatagaagaattaatccccgaaaatcgctccgagtaccgtagcagggcttacgatttatgcactgcaacaaactacgagtgcttgtacgactatgccatgacttacaacagggatgttgcccacttttcggccaactacaaagcttccattaaggaacttaaagagacgaatcgcgacaatattgtgtcttgtggcgtcctggaaacgcctcgttttggacgcaaggacacgttcctgtttgtgccaggcaccaaagtcacttttgagtgcagccaggactttattttggtcggggatcagagacgggagtgtttgtcaaccggcgagtggaacataccggaatatggctacaccgagtgtttacgccatcaggagtattcgtcgagaaaggcggccataaccgggggcatagtactagccgtcattataccaatattactaataatcgcgttttgcgcgttcagtttgttcaaacgacttgcgaacgacaaaagttcgtggcaatacaatgctgttaaaagctcacgaccgttgagtccgatgtcggatgatgggtcttcgctccgtagtcccgttccgagtgaccgctcgagtactagcagtttaggcaaaaagaggcgaagttatgataaggtttaccgtactcatgagcccttggaagggttgccggagacagagtttgaggaaaagccgtgggaccccaacgaatttgaacccgacagtaaaccaacaagcccgacttcggtcatttacacccagccctttagtaaacctgacttggtcaagtttcagtcaaatccgaacttggatcggttcacttacaactccgatgattacacactgccggtgaaaaagaaccgaatgagcagacaacaagagtattcgcaacgccaggctggtatcgcctccggtatcgtcctagctgttattatcccactcgtccttttattcgtttatatcgcatatagatttttcgagaaacgacaacaggaaaaggacgaagaggaacaagatgcgttgaattacgaggccaagaaaagggaagcccaagaaattgccgctagaaaattaaccgcaggggagcatttcgacgatgatacggccagtagcatcgtcagcgggggcaaagaaaccacaatcgattga

SEQ ID NO: 77 - DNA, 4364 bp, Diabrotica virgifera virgifera
atctcgttctagcgattcttgcaaacactcgagtacgaactttactaaccgtagcaactttttttgatgtgatgtgcgccaggttgtagtgccaaataaccaaaatgtggttcaagtactttgttatttttgttttgtttagtgttagcgttaaaggactagatggtgatcttccaccagatgtcgaaactatcgatgtggagactacgaatgtaagtaacgcggatccagacgcagggaaacctattccggtggaagatacgcctaatggagtccctccagtgaatacaaactatgatcctatgacctcagacacagctccaccagacactgaccaacgaggaggaggaggaactccatacactatatcagaaccgagactggcagaaataaggaaacattttatgtacccatttttcgataaaggaggcagtgataataacttgggagatttccaaaaggacattcaatcttccattcctcaggtgcacaagaatttaaacttccagttgcctttctttggattcaggtataattacacaagagtttcagtgaatgggtatctggaattcagcgatccacctccaaattatgactatccattagtatttccaattaaagaatggcccaaaaagaatgatcctgcctttattggcattttcttcagcagatgcagaatcggtaatctacgtgatggagacattgatcaaagaacacccggcgtctattttagaatggaaagagatttaagaacgaggcaagacagaatgggtgtagaaattagagaaagacttaagtgggacatcagaacaggagtcattggatcagaaacattcgatcctaagcacgccgttattgtcacttggaaaaatgttactttcactggaggattcgctaatgctaaatacaagacaaacaccttccaaatggttcttgcaactgacgaagtcttcacttacgcgatgttcaactacctcaacttggactggacctcccacactgaagcgggtggtgattcccaaaacggagaaggtggtgtcagtgcctatgttggttttaacgctggaaacggtaccagaagctacgaatacaatccttacagtcaagcttccgtcattcgagatctcacatcagttggatttggaaatggttttaagggaagacatattttccgtatagacgaagacattttgcttggatcgtgcaacaaagatatagatggtgccaaccttcccctgaaattcgctcctgaaagtggtaacatgttgggtggaacagtggtaaatataactggtccatgcttcaatttgaatgataggattcgatgcaagtttgacgtatcaaatgaagtatttgggtatgttgttgataaaaacagagctatctgtgtacaacctcaactttatgccgaaggatgggtgaatcttcaaattgctgttaattccgaagcattcaagtggaaggggaagtattatgtagaatcacctgctagtgcgactcaaaaaatcttctttaaggacatgaaattacacgaaaaatctccgagcgaattaaaaatcacttgggagaagcagaatttgaccacaaacgacaacgctaacatccgaatttctctctggggttacagagaaacgactatgaaaccagtgtttgtttatatcaccgatatcgctgataatgttcaaaacattggagaacacacgattgttccttctcagtttagaactagagtcaatcaatatctgactgatattaagtttggattcctccaaattaatttaactgagtctatcacagttaatacatatactacaagccagagcagcataaatttaacaccggtaatatggagccgacccattccactcggttggtacttccagttccaatgggaaaaccagtacggtaaaaattggcccaaatatctatgtgacgattggctgagaacggacagatatctaaaaaacttcgcccacgaacttgctcaatgtccctgcaccgtcgaacaggctttagctgacaagggcaggtttatgccagacttcgattgcgacaaagattcgaacccgatttgttactataacaaccaagcacttcattgtgtgaaaactggatctcccactttagaaggctccgaacaacaatgttgctatgataaaaacggctttttgatgctctcttacgatcagcaatggggttctagtccgagacgttgccacaacttgggtaaaatgccttacaatgaagcgacaaaagtgcctactctctcacaatggtttaatgacatggtacccaaatatttgtgttgtctttggcaagacgagcaagctgtgggatgtgaaacactgagatttgaacggagacctacacaggattgtgtagcttaccaagcaccaggtgtagcaggagtctacggtgatccacactttgttactttcgacgatgtagaatatacgtttaatggaaagggagaattcgcattggtgaaatcagttacgcaaacggacaatctggaaatccaaggaagatttgagcaaatggaccctaacacttatggtgaggttagagctactcagctgacttctgtagtagctaaaggtaataacacaatagcaattgaagtcagaaggagacctctggattctagatggagatataggttggatgttatagcagataatagaagactttattttgatagaccctcactaaaattccagcacttccaaggtgttactatttacactccaagctacatccttaatcaatcagaagtcatcattatgtttgacaatggagctggaatggaggttgtcgacaatcaaggattcatgagtgcaagagtatttttgccttggtcgtttataaataaaaccataggcctttttggaaattggagttttaacaaagaagatgacttcacacttccagatggttctagagcagcaatagtgaataacataaatgacatggaaagggtttacaatgattttggttccaaatggatgttggaggatgtgttagatcctcaaaagggtagagcattgttccacagggaattcggaagaacttcagcgacttacaacaacaaaacgttcaagcctcaattcctaatgaatccggaagatttcctaccaaccaacagatctacagatcttaagcgaatatccgaaatatgtcctctaaagttgtacgaatgttactacgattacgctatgacggttgacagggatttggcgcattacaccaagaattataaagccacgatctatcaatataaagaaacgacaaggaggaaagttgtgtcttgcggagtactagaaacaccgagattcggacgaaagagtactttcctgttcgtgcccggtacgaaggttacctatgaatgcatccaagagttcgtgttggtaggtgatcagcggagagagtgtcaagcggatggaacatggaatattcctgaatacggatacacatattgcttacgtcaagaagaatattcttcgcgtcaagctgccatcacttcaggtataatcctcgcaattatcataccattggttcttctattaggttacgttggctacaaaatatacgaaagactaacaagtaacaatcaaaactatgactacgacaccgtacaaaagactcaaaatctccaacaattcagtcgaactttaagtccatacagagaagaagacgaggacgaaaagtatcctcaaagcgacactgattcactaagcaagaaacgaagaagttacgacaaatcctacagaactcacgaacctttgcccaatagaccaaattctgagtttgaggaaaaaccattggacccttacgatcaaaccttcgaagacgacagggtttctagaacgcctacgggaagtccaaccagccctacttctagtatacaatatacaacgccttatagtcggccagacattattagagggaacagctcaaagaatttgaactataacgacatgtatgcagagccaataaagaaacctaaagaaaggattagcgctagtcagagtagtatagtgacagacgtttagaatataattagagtagaaatggacttcagtagtttatagagtatcatttaatttataataaattttcattattttactatccgaagcctttaggtattttaagtatttgtgccgctgtacctagaaacattcttcagtcaaaaatttatgttttgttcaattattttttacttttgtaagcatatattacatttatatctatactggctatctctagcatgtggagtgattgttttgttaatttgtggaattataagcaaaattaaaatgtttaagtattgttaaataaaaataaagtatttacaaataaaa

SEQ ID NO: 78 - DNA, 5238 bp, Diabrotica undecimpunctata howardi
cgaactttactaaccgtagctactttttttgatgtgatctttatgttgcgctcatcagtagtgcgccagtttgtagtgccaattaaccaaaatgtggttaaagtactttgttatttttgttttgtttagtgttagcattaaaggacaagacgatggacttccaccagatgtagaaactatcgacaccaatgtaagtaacgcggagccagatgtagggaaacctattccggtggaagatacgcctaatggagtgtctccagtgaatccaaactatgaagtagataaatccctagttaatgtaagcaatagtttggtaaatgtgactaaaaatcccagacggacttatcagacagttaacaaactgggcaaatattcgacattttatgctgcagattacgatcctatgacctcagacaccgctccaccagacactgaccaacgaggaggagggactccatacactataaccgaaacgagattggcagaaataaggaaacattttatgtacccatttttcgataaaggaggtagtgataataacttgggagatttccaaaaggacattcaatcttccattcctcaggtgcacaagaatttaaacttccaattgcctttctttggattcaggtataactacacaagagtgtcagtgaatggatatctggaatttagcgacccacctccaaattatgactatcctttagtatttcctattaaagaatggcccaaaaagaatgatcctgcctttattggtatattcttcagcagatgcagaatcggtaatcttcgtgatggagacgtcgatcaaagaacaccaggggtctattttagaatggaaagagatttaagaaccaggcaagacagaatgggtgtagaaattagagaaagacttaaatgggacatcagagaaggcgtcattggatctgacaccttcgatcctaagcacgccgtgattgtcacttggaaaaatgtttccttcactggaggattcgctaatgctaagtacaagacaaacaccttccaaatggttcttgcaactgacgaagtattcacttacgcgatgttcaactacctaaacttggactggacctcccacactgaagccggtggtgattcgcaaaacggagaaggtggtgtcagtgcctacgttggttttaacgcgggaaatggtactagaagctacgaatacaatccttacagtcaagcttccgtcattcgagatcttacttcagttggatttggaaatggttttaagggaagacatattttccgtattgatgaggaaattttgcttggatcgtgcaacaaagatatagatggtgccaaccttcccctgaaattcgctcctgaaagtggtaatatgttgggaggaaccgtggtaaatataacaggtccatgcttcaatttgaatgataggattcgatgcaagtttgacgtttcaaatgaagtattcggttatgttgttgataagaacagagctatatgcgtacagccccaactgtttgccgaaggatgggttaatcttcaaattgctataaattctgaatcattcaagtggaagggaaagtattatgtagaatccccttctagtgcaacccaaaaaatcttcttcaaggacatgaaataccacgaaaaatctccgagtgaattaaaaataacttgggagaagcaaaatttgaccacaaatgaaaacgctaacatccgaatttccctctggggttacagagaaacaactatgaaaccagtgtttgtttatatcaccgatatcgcagatagtgttcaaaacattggagaatacacgattgttccttctcagtatagaactagagtaaaccaatatttgactgatattaagtttggattcctccaaattaatttaactgagtctatcaaagttaatacatacactacaacccaaacaaccatagaattaacaccggtagtatggagccggcccattccactcggttggtattttcaattccaatgggagaaccagtacggcaaaaattggcccaaaactctatgtgacgattggctgagaacggacagatatctaaagaacttcgctcacgaacttgctcagtgtccctgcaccgtcgaacaggctttagctgacaagggcaggtttatgccagatttcgactgtgacaaagattcgaatccgatttgttactataacaaccaagcacttcattgtgtgaaaactggatctcccactttagaaggttctgaacaacaatgttgctatgataaaaacggctttttgatgctctcttacgatcaacaatggggttctagtccgagacgttgccacaacttaggtaaaatgccttacaatgaagcgacaaaagttcctactctctcacaatggtttaatgatatggtacccaaatatttatgttgcctttggcaagacgagcaagctgttggatgtgaaacactgagatttgaacgtagaccaacacaagattgtgtagcttaccaagcaccaggcgtcgcaggagtctatggtgatccacactttgttactttcgacgatgtagaatatacgttcaatggaaaaggggaattcgcattggtgaaatcggttacacaaacggacaatctggaaattcaaggaagattcgagcaaatggaccctaacgcttatggtgaggttagagctactcagctgacatctgtagtagctaaaggtaataacacaatagcaattgaagtcagaaggcgaccactagattctagatggagatataggttggatgttatagcagataatagaagactgtatttcgatagaccctcactaaaattccagcacttccaaggagttactatttatactccaacctacatccttaatcaatcagaagtcatcattatgtttgacaatggagctggaatggaggttgtcgataatcaaggatacatgagtgcaagagtatttttgccttggtcgtttataaataaaaccataggcctttttggaaattggagtttcaacaaagaagacgactttacacttccagatggttccagagcagcaatagtctctaacataaatgacatggaaagggtttacactgattttggttccaaatggatgttagatgatgtgttagatcctcaaaagggtagagcgttattccatagggaatttggaagaacttcagccacttacaacaacaagacgttcaagcctcaattcctaatgaccccggaagatttcataccagccaacagaactgcggatattcagcgaacatacgaaatatgtcctctaaggatgtacgaatgttactacgattacggtatgacgcttgatagggatttggcacattataccaagaattataaagccacaatatatcaatataaagaaactacaaggaccaaagttgtgtcttgtggtgtactggaaacaccgaggtttggacgaaagagtactttcctgttcgtgcctggaactaaggttacctacgaatgcattcaggagttcgtgctggtaggtgatcagcgcagggagtgtcaagcggatggaacgtggaatattcctgaatacggatacacttattgcttacgtcagcaagaatactcccaacgacaggctggtctggcttccgggataattatggcaattcttataccgctcatcttgcttttcgtctatctagcgtatcgtttcttacaaaaaaagcagaaagagaaagaagctgaaaaattagaagagcagcaatttatggatcaacaaagaagggcacacgaagcagctactaagaagttaacagaagccgaatatgcttcagaagatgaagacagtaatgttacaagcacgggcgcaaaagaaacaactgtatattaactcctataaaatacaatgattcaacctatgagttgcaacattttaaagtttaatatttctaaaatctttatatagaataagtactagatgtagttaatgtataaataggacttttttattgtaggattaaaaatccactgatgactaaattcaaacactatgttttattcaataaagtgtagacttttttatgtttatgttccatgtagagtgtgatgtgtcaaatatggcgtttttaaccaccacgtaatgtcttcttcctatagtaaatttaatattcttcaaaggtacttgtccattgaattgtatgcctatctaatcctgtaatgcccatctttttttttcgaattaaatgaataatacaaaatttgaaaatcaaaataaattaatcaacttcatataacccggtagaaagcgatgtcctgcaactattatctaaattaatatattattcagtaaccatatcaagctctattagtggtgttatactcctgtattttataaacaacggtttgttaatccaccaacttgcttctatttgagaagatgaatcaatacgctcagctgctcacataaaatttcacatagaatcgtttcaagcaagccagagtcatactcacaatttgtcaaaatttttgccgacaaactgtatttagcatagaactatcttatactaggaagatcagaaaatatctatgccatgaacttaagcacaaaactagccaaatgaaacggcttattggaagggatatgaagtggaaaatggatgtacctaattatgctaaaagtttttcacataccaaagcatttcacaaatttcggccgaatattgttatcacagtaattgtatcttgcatcgttagtctgaaatatcatacatcgacaaaattattagatctaaaatcatcctaagctaaacatacagttcaaaaccattgttgcgtggttacaattctagttttgtgtataaacttcgtccacataagtcgaatccggtttgaatctactcacgagtttgcgataatcggacttttagatttatatatgtatctaatccaggattccaaatataataatgtctaaaatgtaattgttcaaaccgaagtaggaatctctaagaccacttaattataaaagtagttatttgaaagagaaggtaatagttacctgcgttcacatatacaaattaattagaacttgaaagaaattcgttgttaaaacacctatgaaaagaaaaattgatttagtcaacgaaggattccttctagaatgttctatgttatttattaagtacaacatattttcctaccttagtaatactttactttattagaaaatctagtacagttcgtgttaaatctcaaatttactattat

SEQ ID NO: 79 - DNA, 4313 bp, Diabrotica barberi
aaaacataactttttgactgaagaatgtttctaggtacagcggcacaaatactttggtcttaagatagtaaaataatgaaaatttataaattaaatgatactctataaactattgaagttcacttctactctaattatattctaaacgtctgtcactatactactctgactagcgctaatcctttctttaggtttctttattggttctgcatacatgtcgttatagttcaaattctttgagctgttccctctaataatgtctggtcgactataaggcgttgtatattgaatactagaagtaggactagttggacttcccgtaggcgttctagaaaccctatcgtcttcgaaggtttgatcgtaagggtccaatggcttctcctcgaactcagaattcggtctattgggcaaaggttcgtgagttctgtaggacttgtcgtaacttcttcgtttcttgctaagcgagtcagtgtcgctttgaggatacttttcgtcctcgtcttcttctctgtatggacttaaagttcgactgaattgttggagattttgagtcttttgtacggtgtcgtagtcatagttttgattgttacttgttagtctttgatatactttgtagccaccgtaacctattagaagaaccaatggtatgataattgcgaggattatacctgaagtgatggcagcttgacgcgaagaatattcttcttgacgtaagcaatatgtgtatccgtattcaggaatattccatgttccatccgcttgacactctctccgctgatcacctaccaacacgaactcttggatgcattcataggtaaccttcgtaccgggcacgaacaggaaagtactctttcgtccgaatctcggtgtttctagtactccgcaagacacaactttcctccttgtcgtttctttatattgatagatcgtggctttataattcttggtgtaatgcgccaaatccctgtcaaccgtcatagcgtaatcgtagtaacattcgtacaactttagaggacatatttcggatattcgcttaagatctgtagatctgttggttggtaggaaatcttccggattcattaggaattgaggcttgaacgttttgttgttgtaagtcgctgaagttcttccgaattccctgtgaaacagcgctctacccttttgaggatctaacacatcctccaacatccatttggaaccaaaatcattgtaaaccctttccatgtcatttatgttattgactattgctgctctagaaccatctggaagtgtgaagtcatcttctttgttgaaactccaatttccaaaaaggcctatggttttatttataaacgaccaaggcaaaaatactcttgcactcatgaatccttgattgtcgacaacctccattccagctccattgtcaaacataatgatgacttctgattgattaagaatgtaggttggagtgtaaatagtaacaccttggaagtgctggaattttagtgagggtctatcaaaataaagtcttctattatctgctataacatccaacctatatctccatctagaatctagaggtctccttctgacttcaattgctattgtgttattacctttagctactacagaagtcagctgagtagctctaacctcaccataagtgttagggtccatttgctcaaatcttccttggatttccagattgtccgtttgcgtaactgatttcaccaatgcgaattctccttttccattaaacgtatattctacatcgtcgaaagtaacaaagtgtggatcaccgtagactcctgctacacctggtgcttggtaagctacacaatcctgtgtaggtctacgttcaaatctcagtgtttcacatcccacagcttgctcgtcttgccaaagacaacacaaatatttgggtaccatgtcattaaaccattgtgagagagtaggcacttttgtcgcttcgttgtaaggcattttacccaagttgtggcaacgtctgggactagaaccccattgttgatcgtaagagagcatcaaaaagccgtttttatcatagcaacattgttgttcggagccttctaaagtgggagatccagttttcacacaatgaagtgcttggttgttatagtaacaaatcgggttcgaatctttgtcgcaatcgaagtctggcataaacctgcccttgtcagctagagcttgttcgacggtgcagggacattgagcaagttcgtgagcgaagttctttagatatctgtccgttctcagccaatcgtcacataaatatttgggccaatttttaccgtactggttttcccattggaactggaagtaccaaccgagtggaatgggtcggctccatactaccggtgttaattctatgctgctctggcttgtagtatatgtattaactttgatagactcagttaaattaatttggaggaatccaaacttaatatcagtcaaatattggttcactttagttctaaactgagaaggaacaatcgtgtattctccaatgttttgaacattatctgcgatatcggtgatataaacaaacactggtttcatagttgtttctctgtaaccccagagagaaattcgaatattagcgttgtcgtttgtggtcaaattctgcttctcccaagtgatttttaattcgctcggagatttttcgtgtaatttcatgtccttgaagaagattttttgagtcgcactagcaggagattctacataatacttccccttccacttgaatgcttcggaattaacagcaatttgaagattcacccatccttcggcataaagttgaggttgtacacagatcgctctgtttttatcaacaacatacccaaatacttcatttgatacgtcaaacttgcatcgaatcctatcattcaaattgaagcatggaccagttatatttaccactgttccacccaacatgttaccactttcaggagcgaatttcaggggaaggttggcaccatctatatctttgttgcatgatccaagcaaaatgtcttcgtctatacggaaaatatgtcttcccttaaaaccatttccaaatccaactgcagtgagatctcgaatgacggaagcttgactgtaaggattgtattcgtagcttctggtaccgtttccagcgttaaaaccaacataggcactgacaccaccttctccgttttgggaatcaccacccgcttcagtgtgggaggtccagtccaagttgaggtagttgaacatcgcgtaagtgaagacttcgtcagtggcaagaaccatttggaaggtgttggtcttgtatttagcattagcgaatcctccagtgaaagaaacgtttttccaagtgacaataacggcgtgcttaggatcgaatgtttctgatccaatgactcctgttctgatgtcccacttaagtctttctctaatttctacacccattctgtcttgcctcgttcttaaatctctttccattctaaaatagaccccgggtgttctttggtcaatgtctccatcacgcagattaccgattctgcatctgctgaagaaaataccaataaaggcaggatcattctttttgggccattctttaattggaaatactaatggatagtcataatttggaggtggatcgctgaattccagatacccattcactgaaactctcgtgtaattatatctgaatccaaagaaaggcaactggaagtttaaattcttgtgcacctgaggaatggaagattgaatgtccttttggaaatctcccaagttattatcactgcctcctttatcgaaaaatgggtacataaaatgtttccttatttctgccagtcttgtttctgttatagtgtatggagttcctcctcctcgttggtcagtgtctggtggagctgtgtctgaggtcataggatcgtaatctgcagcataaaatgtcgaatatttgcccagtttgttaactgtctgataagtccgtctgggatttttagtcacatttaccaaactattggttacattaactaaggatttatctacttcatagtttggattaactggagcgactccattaggtgtatcttccaccgggataggtttccctgcatctggattcgcgttacttacattggtagtgtccacatcgatagtttcaacatctggtggaagaccaccatctagtcctttaacgctaacactaaacaaaacaaaaataacaaagtactttaaccacattttggttatttggcactacaatctggcgcacatcacatcaaaaaaagttgctacggttagtaaagttcgtactcgagtgtttgcaagaatcgctagaacgagat

SEQ ID NO: 80 - DNA, 5020 bp, Leptinotarsa decemlineata
ataacaaattttatcttgttacagagcacacttgtgagaaggcaatcgagtaacgaacctcatacataaaagtacacgtatggtgatagaaaacaataaattatcaaactgaggctctgtgccaagttttggaaaatgtacgtcaagtggaaattggttttgactctagtactgtgtgttggtgtcgtgattggggaagatatttcaacagatataatccccttgccacaagacaatacagcagatgtggaaatagtggctacagaaactaggtcaaattcaggatctgaagctgctgtagaagagcccaccaataacactgggccttcagatactacaaacccaaattatgctgtagtgccccctgtagtttctgatagcaaacctgcaataacaaatagtgaaacaaatgacactaacaatgatgttgtaatgttgagtcctacgaaatttctcatgaaaaagggacgatctggacgtttattggaatatccgactgattacgatcctatgacttcccatattgctccaccggatagtgatcagagaggatattcaggagtaccatatgtcttgacggaaaccagactgcaacagatccgccaaaatttcatgtatccctactacaacagaggcggtaatgcagatgacgaaggagactaccagaaagaaattcagtcatctattccgcaagtgtacaagaacctcaacttccaactccctttcttcggatttcgattcaattacacgagggtctccttaaacggctatttggaattcagcgatcctcctccaaattacgactatcctttggtctttccagtcaaggaatggcctaaaaagaacgacccttctttcatcggtatctttttcagtaaatgtagaatcggtaacctgagggacggagatattgatcaaagagaccctggagtgtactttaggatggaaagggatctcagaaataggcaggacaggatgggagtggagatcagagaacgactgaaatgggatataagggaaggggtgatagggtcagaaacattcaatcccaaacatgccattatagtcacatggaaaaatatctctttcaatggaggttttggcaatgctctctaccagactaacactttccaaatgatcctcgccactgatgaagttttcacctacgccatgttcaactacttgaatcttgactggaccacccacactgaagcgggaggcgacacaagaaaaggagaaggaggagttcccgcttttgtgggattcaacgctggaaacggtactagaagttttgaatacaaaccatacagtcaagaatctgttattcgagatctcacacaaactggtttcgctaatggtttcaaaggaaggcacattttccgaatcgacgaaaatatcctaactggaacatgcaataaagatatagatggtgctaatctaccgctaatgatatctccagaaagtggaaatatgctgggtggaacaatagtgaatataacaggaccttgtttcggcctagacgaccaagttaaatgcaaatttgatgtagcaaatgaaataaatggcgtcgttatagataaaaacagggctatatgcatccaacctagactgtatgccgaaggatgggtgaatttacaaatagccataggggctggggtatacaaatggaagggaaaatattatgtcgaatcccccgcagcggcatctcaaaaaatctacttcaaggacatgaaggttcatgaaaaatcgcctagtgaaataagaataacttgggaaaaatacaacttgaccactaacgaaaacgctaacattcgtatctccttatggggttacagagaaacaacaataaggccaacgttcgtttacatcactgatatcgcagacagtctccaaaatactggagagtataccatcgtaccgtcccaatatagaacaaaagttaatgagtttctcacggatatcaaatttggtttcttgcaaattaacttgactgaatctatcaaagtgaacacttatacatctgtacaaagatcggtggaaatagttcctgttgtatggagtcgacccattcccctaggatggtactttcagttccaatgggaaaatatgtatggacgaagctggcccaaagcactctgcgatgactggctaagaacagacagatacctgaaaaactttgctcatgagttacctcaatgcccttgcactgtagaacaggctttggcagacaaagggaggtatatgcccgactttgattgcgacaaggactcaaatcccgtatgctactacaataaccaagctctgcactgtgtgaaaacaggatcaccaacgttggagggatcagaacagcagtgctgctatgacaaaaacgggtatctcatgttatcatacgatcagcagtggggttcaagtccacggcgttgccacaatctgggaaaaatgccctacaacgaagcaacaaaagttccaaccttatcgcaatggttcaacgatatggtaccgaagtatctttgctgtttgtggcaggaagaacaggcggtgggttgcgaaacgctgagattcgaaagaagaccaactcaggactgtgtcgcgtaccaagctccagggattgctgggatttacggagatccccacgtcatcactttcgatgacgtcgagtacaccttcaacgggaaaggagagtttgctcttgtgaaatctgtgacacaaactgacaacttggaggtgcaaggcagatttgagcaaatggaccctaacgcctacggagaagtacgtgcaacacaattgacttcaattgtggcaaggggaaacaacaccatagcagtggaggtcagaaggaggcccttggatgctaggtggaggtataggctggatgtcatagctgataataggaagttgttcttcgacagaccctctttgaaatttcaacatttccaaggagtgactatttatacacctacttatatcctcaatcagtctgaagtcatcattatgtttgataacggagcaggagttcaagtaatggataaccagggattcatgaccgcgagggtgtatcttccttggtcattcatcaacaaaactgttggtctctttggcaactggagtttcaataaggaagatgacttcactcttcctgatgagtcgaaggctgccgtcgtgagtaatatcaatgatatggaaagggtctacaatgactttggttccaaatggatggtggacgacgtactagatccgaaaagaggtagatccttatttttcagagaattcggcagatcatcggcaacgtacaacaacaaaactttcaaaccgcagttccttatgttacctgaggacataatacccgcaaacaggtcgatacagatacagagaacttacgacatttgtagcacaaaaatgtacgaatgcttctacgattatgccatgacgctcaacagagatcttgcccattttactcagaattataaagcaaccatatatcaactcaaagaaacgacgaggcagaaggttgtttcttgcggagttctggaaacaccgcgattcggtaggaagagtacttttctttttataccaggaaccaaagtcacttacgagtgcaatcaagacttcgtattggtgggagatcccagaagagaatgtttggcagatggcacatggaatgctcctgaatatggctacaccgaatgtttacgtcaacaagaatattctcagcgccaatcagccattgcctctggagccgttctcgcaataattattccactagttctattatttgtatatctggcttatatgttcctcaagaagaaacagaaagaacgagacgaagaaaatttacaaacgcaagcgtacgagcaacagaaaagacaagctcaggaagctgctgctagaaaattaactgctgccgaagagtacaactcagatgaagacgataataacagcaacgttacgagcacagcaaaagaaacaacagtgtattagtattagatatggtaacacttttacgatattattattttggttggcatacttctcagacaaacagtccaaattattatttgcaacaagaactatttgaatttcgaagtttcctttgaagatcccaagttgcttattatcctaaatttttgcaaaatgtcaaaatcgtactattccatcagttgtatattttatttttctgagatagttaggccaacaggtatctaaaggagtgaataacagatttttcatattttccagttttatctgattcaataatgcactatgggttttttctgttaatggtttttataacacctctgttgcaaaatacatattaggtaatataaatatacgtaattaagtcgtaattaagtattataacaaaatggcgttctctattatttaaattgtaattagatatatattttttatattcataatcacttttgcatgtaaagtaggagtccaattttctgtacttcaaccatcactatcgctacttaccccgatcactttttggatgcacatttcatgcacatttttgcacattgtttgcacagtaagttcactttgcaaacaatgtgcgaagactatgcgggaggacatgtttatgcagtaattttgtatatatgtgagtaggtatatacctactattttcatcatactctggtcctacttttcttatttacagtgataagatgatgtttgaaatcttgaattaggtacaatgaattcatttttgtcagttatttgaaacattttcggatattttaataagactgtgttttatcaaagttcagtttgattgtccaaatactagtagatcattagagtcctactgtagttttagcattttaataaagtaattcaatattaatatacaagtaatcttgcacttctgctaactcaaaattttgcaaaattattcactcgaatcagtattcacacgtttctctttcagaaataaccaaaatatttcaccccatacacaaatttcattaggtttagtgaaatttgtgtatggggtgaaatattttggtta

SEQ ID NO: 81 - DNA, 4851 bp, Helicoverpa zea
caacgctctagtcgaccagtacaatagcgagccacctgttgtcgtcaaaactgataaaaaataatgtgatgaacattttgaaagtgtgaaaatgggtgttaaagttctagctttaatagcactcttagttattagtgttaatggacaagaaatagttgacaacgtcacaagtaatgttgctgaggacaatatagtaagtgatactacgacagtggtggtaccagtaactgaagtaaaggaagatgagattgataatgaaagtccagtggaaattataagtgacaaagcagaactgcaagttaggagtgggaagtaccagacattgactgatggactggggggcgaagagccgctggctttggatgcggttgatttaaacgtcaacgataatttgctcagtgaaaggcagttattgtcaccaagcaccactcaagttacgaacaatgagtatgctcacatcgacggccgtgtgctacccgacacgagctaccagaacaatggtcagccctacgtcatcacagctgccagactggcgcagatcagaggcaacttcatgtactggttctacgatcaaggaggcaacgagaatattggagactaccagagagacatccacacgtctactcctcaaatccataagaacttcaacttccagttgccgttctttggatttaggtttaattataccaggttatctatgaacggttacatctacttcagcgaccctccagaccactacacttaccccctctccttccccgtgagagactggcctgatgtcaacgacccctccttcattggtatattcttcagcaagtgccgtatcggtagtcagcgtcctgaggacccagatcagagaaggcctggaatttactttagaatggacagggatttgcagacacgtaccgaccaactgggtgtggagatgagagagcgtttgacgtgggacattcgtcagggtgtcatcggttccgagactttcttccccaagcacgccatcactatcacatggaaaaacatgtctttcgctggaggcattgacaactcgttgtttgtgacaaacacattccaaatggtactggcaactgacgaagtgttcacatacgcgatcttcaactatttggagatcaactggagttcacacacagaagctggtggtgataccactaccggagaaggtggtgtacctgcttatattggtttcaatgctggtaacggaacacaaagctacgaatacaagccttattcacaagcgtctgtacttagagatttgacgggcagaggatgggcgaacggttttccaggtcgacatatatttaggatagacgaaaaaatacttatggggacttgtaacaaggatatcgacggtgcaaatcttcctctcatgttcgcccctgagagcggtaacatgttgggaggtacaatcgtcaacatcacgggtccttgcttcaaccccaacgatagaatcacctgtcgtttcgatactgagtccgtcattggtgctgtagtggacgtcaatagggctatttgcgtccaacccaggttctggcacaatggatatgctagattcgaaattgcgattaataatgaaccctataagtggaaaggaaagtactttgtcgaaacacccgcaacggccacagagaagattttcttcactgacaactcagtccacgagagatatcctcctgaaatcagaataacttgggaccgcttcaacttgaccacgaatctaaacgtccaactccagatcagtttgtggggctacaaggaggtcactatcagacctcaactcgaatatattgacatgatcgaaatgggcgtggctaacaccggcgaatacgtcatcaatccacaaaactttaggaacagggataacttccttcacaatgacatgcagttcggtttcctacaaatcaacttgaccacaccagaagtgtttaaaaatgttgagatatcacctgtcctgtggagtcgtccaatccccttaggttggtacttcgccccacaatgggaaaggttacacggccaacgttggcccaacgccctttgtaacaactggttacgaacggatcgtttcttgaagaactttgcatctcaagtatgggtttgcccttgtacgctggaacatgcgttactggataagggaaggtttatgccagatttagcttgcgataaagacaccaatcccacctgtagataccattggggcagtgtccattgtgtcaggagtggagcacctagcgcggaaggatcaggtcagcagtgctgctacgacaagaacggcttcctcatgttgtcgtacgatcagatgtggggatccaggccttcgaggtctcacgacttcgggttcactccttacaacgaggccaacaaggtcccatcgctgtcccattggttccacgacatgattccattctaccagtgctgtatgtggcaagaagaacaagctgttggctgtgagacattcaggtttgaacgtcgtccttctcaagactgtgtagcttaccaatcgcctggtgttgctggaatatttggagacccacacattgttacattcgatgacttgcagtataccttcaacggaaaaggtgaatacgtattagtaagagtggatcacccgcaactaaaactggacgtccagggcagatttgaacaagttccacgtaacatctacggacgagtcaacgctacacatctgacttctgtcgtcgctgcttccaataactccgtacctattgaggttcgtctccgtccccaacatgctcaatggcgttatagacttgatgtattcgctgacaacaagagagtgtacttcgacaggcctgcccttagggtgcaatacttcccaggtgtgacagtataccagccaatgtacttactgaaccagtcggagattgtcatcatgttctcatcaggcgctggtattgaagtggtagagaataaaggctttatgtcagctagggtttatctgccttggaattatatgaaccaaacgcgaggtctattcggcaactggtctctggacattaacgacgacttcacgcgaccggacgggacgcgagccacagtcgacctcaacaacttccaaactgcacacagggacttcgctcaatactggcaactaaccgaccgcgaacaacgagacataggagtagcgatgttctaccgcgaatacggcagaacggcagcctactacaacgacaaccaattcataccaaacttcatcagggaaccagctgacttcctaccagcgaacaggtctcaagacgtggcgagagctgtggaactgtgccaggattcctatcaatgtcgatatgattacggcatgaccctcaacagggatatggctgagttcacgaagaattatttgtcttctatcacgaatataaaagagcagaacgctcgaagagtgatcagctgcggtatattggagacaccacgatttggacggaagagtaacttcttcttcacgccaggaacaagagtgaacttcgagtgtaaccaagacttcattctgattggagacaagcgacgagtgtgtgaggacaacggccggtggaacttaccggattatggatatactgagtgcttgcgtaaccaagaatactcgcaacgttctctgtttctgacctgggcgatcatactggcagttattctaccgttaggcctgttgatttgtctgctgtggttctggtgctggtacaaacccaggaccgaaggcaaagaaggattccgtttcgaagatattccgcgatcaaaatcagcctctagacttaatctaagatcagcatcaatgggaaacctcacggatacgatgaaatcatccacattacgaagctccgactctaagcctaagttaccagaaactcctactgaggaagccccaatgactaggtctgcacctctagttaggccagctccacccgtcccacaagacggcgatagttcaggaatagggtacccagattccaataagagtgacaaatctgacaaatcaaatcctaaaaaacgtagggcctatgacaaaacttaccgaaccaatgaacctatcccgaacgctcccaatgaagaattcccagaaaaactgtgggatctttccgaagaggacttgctttctataacttcaccgtcagacactgaatctaatagagattcaacgttgacgcgacctgctaaagacattgaatatcaaagcagacctcgccagacaggtcgtagagcactgccaagtgactcgggttattccacaaaagattccgaggacccatactctccgaagtatgaaggtcagtacagtcctattccttcccagtactctccaacgtactccgaaatatattctccaccgatcagccctacttcggacaatagtccaagaaacacgttcaataaccccggtttgcctgaccctccgaaaagcgctcctgcagattctattcagacgtttaccatgcctccccaaaaggggaaatctttggagactctgattgacccaccgaacattgaagtgcccacaatgagccctcgctcgaccatggtctgaaacacttagaaataaaaaactagtaaacaggaatttagtatattcactgcccaaatacgacattagacctgtcgtcctgtatggatcggagtgttgggcattgaaaaagagggacgagaagagagtgcatgtaacagaaatgagaatgctgagatggatgtgtggtgttacaagaatggataaagtgaggaatgattacattagaggaagtctgaaagtagcgccagttacagaaaagatgagaagtagaagattgtcgtggtatggacatgtaatgaggagggatgatacgtatgcaacaaagt

SEQ ID NO: 82 - DNA, 7269 bp, Spodoptera frugiperda
tctgccacctgttgtcgtgaaaattgataaaaaataatgtgatggacattttaatataagtgtgaaaatgggtgttaaggttttagctttaatagcgctcttagttgttagtgtactcggacaagacgttactgtcagtgacaacgatgtaacaaaggaagttgtggtggacaatttacctagtgagaacccgatagtattagacacagttacagaagccacaaaagatgaagttgctgaagatgcgaacccagtggaaatactaagtccaacggatgatctgcaagtaagaagcgggaaatatcagctcaatgatgggctcgtgggtgaagagccgatgccactagatgctgttaatttcgactcgaataatgatgccggagagagtgagaagcagttgctctctcctggcacaactcaagtcacgaacaacgagtatgctcatatcgatggccgagttttaccagacaccagctatcagaacaatggccaaccctacgtcatcacggctgctcgactggcccagatcagaggaaacttcatgtactggttctatgatatgggaggtaacgagaacaatggagattaccagagggacatccacacttccactccacagatccataagaacttcaatttccagttgccgttcttcggatttaggttcaattacaccaggttgacgatgaacggctacatctacttcagtgaccctccagaccactacacataccctctctctttccccatgagagactggcccaacgtgaacgatccgtccttcatcggtatcttcttcagcaagtgtcgtatcggtagcctgaaccaggacgaccctgatcaacgaagaccaggagtttactttagaatggacagagatctgcagactcgtacagaccaactgggagtggagatgagagagcgtctgacatgggacatccgcgagggtgtgatcggttccgagaccttcttgcccaaacacgccatcaccatcacctggaagaacatgtccttctctggaggaatcgacaactctctgtttaggacaaacacattccaaatggtgctagcgactgatgaggtgttcacgtacgcgattttcaactatttggagatcaactggagttcgcacacggaggctggaggtgataccactacaggagaaggtggaacgcctgcttatatcggattcaacgctggtaacggaacccaaagctatgaatacaaaccctactcacaagcatctgtgcttcgagatctgacgggcagaggttgggccaacggtttccctggacgacacatattcaggatagacgagaaaatacttatgggaacttgtaacaaggatatcgatggggcaaatcttccgttgatgttcgctcctgagagcggtaacatgttgggaggtactatcgtcaatatcacgggtccctgcttcaaccctacggacagaatcacttgccgtttcgacacggagtccgttataggtgccgtagtggacgccaacagggctatatgcgtccaacccagattctggcacaacggatacgccagatttgagatcgccatcaataacgaaccttataaatggaagggaaaatacttcgtcgaaaccccagcaacggctgcagaaaagatattctttactgacaactcagtccatgagaggtatcctccagagataagaataacttgggaccgcttcaacttgacttcgaacctcaacgtgcaacttcagatcagtttatggggctacaaagagattacaatcagaccccagcttgaatacatagatattatcgaaatgggtgttgctaacacgggagaatatgtgatcaatccccaaaactttaggaacagggacaacttcatgcacaatgacatgcagttcggtttcctccagattaatttaactacgcctgaagtcttcaaaggagtctcaatctcgcctgttctttggagtcgaccaattccattgggttggtacttcgccccacaatgggaaagactgcacggctcacgttggccgaatgccctttgcaacaactggctacgaacggatcgtttcttgaagaacttcgcttctcaagtctgggtctgtccatgcacattggaacacgccttattggacaaaggaaggtttatgcctgatctggattgtgacaaagacacgaaccccacttgcagataccactggggaggtatacactgcgtcaggagtggagcacccagtgcggaaggttctggccagcagtgctgctacgacaagaacggtttcctcatgttgtcctacgatcagatgtggggatccagaccctcaagggctcacgacttcggattcactccttacaacgaggccaacaaggtcccatcgttatcccattggttccacgatatgataccattctaccaatgttgtttgtggcaagaagaacaggcagttggctgtgaaacattcagattcgaacgtcgaccttcgcaagactgcgtagcataccaatctccaggagtagcaggaatattcggagacccacacatcgtcacattcgatgatttgcaatacactttcaatggaaaaggtgaatacgtcctggtacgagtggaccacccacaactgaagctggacgtccagggtaggttcgaacaggttcctcgcaacatccacggcccggtcaacgccacacatctcacatctgttgtagctgcttccaacaactccgtacctatcgaggtacgtctacgcccccaacacgctcaatggagataccgactcgatgtgttcgcagacaacaagagggtctacttcgacaggcctgctctccgagttcaatacttcccaggtgtgacagtgtaccagccgatgtacttgctgaaccaatcagagattgtgatcatgttctcgtcgggcgccggtgtcgaggtgatcgagaacaaagggttcatgtctgctagagtctacctgccttggacttatatgaaccaaactcgtggtctattcggcaactggtctctagacgtaaacgatgacttcacgcgacctgatggcacgcgagtcgccgtggacctcaacaacttccagactgcacacagggacttcgctcagcactggcaactaaccgacagagaacaacgagacataggagtggcgatgttctaccgagaatatggtagaaccgcagcatactataacgacaaccaattcgttccgaacttcataagggagccggcagacttcctccctgctaatagatcgcaggacgtggccagggcgatagaaatctgccaagactcctaccaatgccgatacgactacggtatgacactcaacagagacatggctgagttcaccaagaattatttgtcttctattacgaacattaaagagcagaacgcccgtcgagtcatcagctgcggtatattagaaactcctcgattcggaagaaagagtaacttcttcttcacaccaggcacaagggtgaacttcgaatgtaaccaagacttcattctgattggagacaagcgtcgagtgtgtgaagacaacggacggtggaaccttccagactatggatacaccgagtgtctacgtaaccaagagtactctcagcgcgcactgttcttgacctggggcgtcatattggcagttattctaccgttaggcctgttgatttgcctactgtggttctggtgctggtacaagccccgctccgaaggcaaagacggattccgattcgaagatattccgcgatcaaaatctgcttccagactaaacctaagaacagcctcgatgggaaatcttacagacacaatgaaatcttccactttacgtagcacagattctgataaaaaggctaaattgcccgaaactcccactgaggagaccccaatgactagatctgctccattaactagggccgccccacctcccccacaagatggtgacagttcaggcatagggtacccagattccaataagagtgactccaataaatctgacaagtcgtttaaaaagcgtagggcctatgacaaatcctatcgaaccaacgaacctatcccgaacgctcctaacgaggaattccctgaaaaactttgggatctatctgaagaagacttactttccataacatcgccttcagataccgaatcaaacagagattcaactttaactcgtccggcaaaagatatagaatacgttggcagaccacgtcagttaggtcgcagggcgttgcctagtgactcaggttattctacaaaggattctgaagatccttactctccgaaatacgagggtcaatacagccccataccttctgcgtattctccaacttattccgagatatattccccaccgatcagtcctacctcagacagtccgaggagtacattcaataaccctggcttgccagaccctccaaagagtgctcctgcagattctatacagacattcaccatgcccgcacaaaagggcaagtccttagagactctgatagacccacctatttccgaaatgcccacaatgagccctcgctctaccatggtctgaataaccttcaaagtattctatacattattcactgcccatccaatgacattgccaggagtattataagcttaggaataatttattacttaagaagcacattattttagtattttgcacttaatgttttacataattaggttttatttaggttctaatggatttccgtgagtttcccatcgttgttgttgttgttgaatgtttgtaaattctattaaatgattaatttaataaaacttattttaaatatgtctacgcagtcacacgaagaggaaatttctgttcccatttcggtagaataacttgatcaattgattttgttttaggtaagaaagcaaggaataatgtcacgggccacgatgtgtggaatagaaattttctctgacttgtgatgagttattattttattcttcctaatctgtaaggttattgcttgctttcttatctagtgtacataaaaaaaccaggtcatatgaacaggtacgtgtattaatttttttatttgcattacggttatcttttagggctttgcgcagccgatagcgtatctttgttttttgataaattgcgttataagggctgtgtattattttttcgtcgttgggaaatggtattggctgataagatttttatgtaacgaggttattcaaatacagacagtgttaacttttatataaatcgatgggatttgttaatatgcaaaggggaggaagtggggcattcgattagttaagtaggtaccttaaatgtttagttaatcaattagaaagccagaaagcatcaattaaaatcataaacattaaagacaaaaggaaatctcatagttagttcaatttctgcacggaaactgcacggttggcgcggtgaccgggcaaccggctgccgcgaaacgtgtagcgggttcgattcccgcacggagcaattcttcgtcgccaaacttcaccgctgtccaataaatttacttttgactgcgtagttggggcggtagctgggcaactggttgccgtacaacgtgtgtagcaggttcgattcccgcacgcagacactctttatgtgatccacaaattgttgtttcgggttctgggtgtcatgtgtatgtgaacttgtaaatatgtttgtgaatgcaccgacaacaggggagaactagggtaggacaatgtttaaaaaaaaacaaatttcagactgaacatccatatgaatcctcctttcttcaatgtaccctatacaatacgctaagcataccagaacattcttaacagcaccattattttgaaaaataaaatcaacatttactttacttttctaggcccctggaccctgaggaatcccttcgaatcaacggtgtcatgtatatgtgaacttgtatgtttgtaaacgcaccgacgacacaggagaaaatgctagtgtggggtaaagtttttcaaataggctaaagaaacacaggtaattaacacattctaaataatacaggtaagcaagagtactcgtcccgtacactgggagcgacatggggcatcatcagtgctatcatgatacccatcgtcattgtgctgatatgcatcggatggaggctgctactgcgcaagaaggctgaggaaaaggaggacagggagttcatgaatatcaagcccctggaccctgaggaatcccttcgaatcaactctgatgatgagagcataccgtacaagaaggatacatcagacgacactccggagccaacggaaccagtgaaggtggataatcaagaaccttcgccggagcctattccgccggcgcccatgccagcacctttgccagtagtgggcataccatacccagcatatggcatgccttatgaccagagcagacaagctcggccgtacggtggcgaaacggaaattaactaggttaagactaccaaatgtagcgtagccaccaaaaatgtatttagtaattgggccaaaagaccagtatctaaaccataaagtgttgccacgctatgcttgtttatggtggaaattaaatactgaaataagttttcaatatgtttgaaaaattattcagtcttattttttcagactaaataataagatacttataggtttacttggataaaaaaataaggttgcatttttaataataaaagttatatttcgtggttctattttgtacaaaaacgtcctagaatatatttgtttttttatatacttacctaagcttatataataactactttgtctctaatcagatgacagtacgttctttgcctaccgtaggaattaagtatattaaaaaaatccataaaaaaactaataagctcaaactagatttataaactaataagctcaaactagattcatattgaaaaaacgtgttataatttggttttactgcaaacaaaaacctagctagctaggtaaaactagcctagctaaaacttgtgcagaatatatttttatacattttaagcaatttataagaattttaaatattatgtaatactaaacaatatgaataaatagttcccgttgccataaatcaacattaactttggaatttattcactcaaaattcggaatcaatgaataatcccaccattgaaataattctaggtttaagtaaataggtatgttgttaaataagtacaatacctgttttgttttacatgacaacgattcagataaaataagcattctgacattcatattcatgtacctagttagttgaataaaattttataatattaaaaaaaa

SEQ ID NO: 83 - DNA, 5316 bp, Ostrinia nubilalis
cgcgatacagagcaattgtagcaacaatgtttgaagttgaaagatactgttgtgtcctaatttagcactaaaattcataaagcgttttaaataaatatgactcgcaagtggcagttttgtcaaaattacatttaataaataaatccgccgcaaaatattactttttaaaattacctacttttggataaccattgataaccttttggacaaaaacatacttacaagaatactaggtatttaagtgataacaattaagacgaaacaaacagctttaatcgacattagtaataaaccaactgtcgttcaaattgtacactctccgactcgttgaagtacgaagtagcacctcaaaaacagagcaagtaaattcatatagggttacgtggattaggacagagaatttggcgcacctaacgttcctatttattatcttatcatatctctagctgataacaccattatcaatgagcacacaaagcacctcactccagtcgaccactacaattgcgaggcacctgttgttgcagaaaaaactgataacggaactaaagaaaatgatgtgatgaaagataaaaaaatcgattgaacaatgggtgttaaagttttagttttagccgcgtttctagttataagtgcaagtgtatacgctcaggacgttgtagacaatgtgacagacagaactgttgatgaattattattagatgtggacccaaaattagaagttacaccgccgacggaagaattgccgcagccaaatgatgaagtgcctgaagaatcggcagttgaagttatagcagatgttccggcagctgatttggagatccgaagtgggaagtaccagctgaacgatggcttggtgggtgaggagccagtggaactggaagcggtggccaatgtcgaaccggacccaaatgctgagcagagtgagaggcaactcctatactcacctgactctacaatgaacaccaattataataacgaatacgcacgcatcgacggaagaatactccctgatataacctaccagaacaatgggcaggcttacatcatcaccgcccagcgcctccagcagatcagggctaacttcctctactggttctacgaccagggcggcagcgagaacatcggagactaccagagggatatccacacatccaccccgcagatccataagaacttcaactttcagctgccgttctttgggttcagattcaattacacgaggctctccatgaatgggtacatctacttcagcgatcccccagaccactacacctaccctctctccttccccatgcgggactggccgaaagtcaacgatccttcttttatcggtattttcttcagcaagtgccgtatcggcagcatcagacccaccgacattgaccagagaagagctggcgtttacttcagattggacagggacttacagactcgtagagaccagcttggcgtagaaatgcgcgaacgcatcacctgggacatccgtgaaggtgtcattggcgctgagaacttcttcccgaaacatgctatcacgataacttggaagaacatgtctttcgccggaggaattgacaactctctctttgtgactaacacgttccaaatggtactggcgacagatgaagtgttcacatacgcgatctttaactatcttgaaatcaactggagttctcacacagaagctggtggtgataccaccacaggagaaggaggagtgccggcttatattggtttcaacgccggtaacgccacccgaagctatgaatacaaaccatactctcaagagtctgtactccgtgatttgactggaagaggatgggctaatggtttccctggtcgacatatatttagaatagacgaaaatatactcatgggaacatgtaacaaagacattgacggtgcaaatcttcctctgatgttcgcccccgaaagtggtaacatgttgggaggtacagtggtgaacatcactggcccttgcttcaaccccaacgaccgaatcacttgccgtttcgatactgaagccgtggtgggtgtcgtcgtggacgtcaatcgagccatctgtgtacaacctcggttttaccacaatggttatgccagatttgaagtcgccatcaacaacgagcctttcaaatggaagggacgcttctttgtagaaacccctgcaacagcaaccgaacgaatctacttccctgacaacgctatccaccagaggaatccaccggaaatcaaaataacatggaaccgtttcaatttgacgaccaatttgaacgtccagcttcaaatcagcttgtggggctacaaagaagtcaccatcagaccccaaatggaattcatcgatatcatagaaatgggtgtggcaaatactggggagtacatcatcaacccacagaacttcaggaatagggataatctgatgcacaacgacatgcaattcggttttctgcaaatcaatttgacaactcctgagatatacaaaggcgttcaagtttcaccaattctctggagtcgaccaatccctctagcctggtacttcggccctcaatgggagcgtctccacggaacccgctggcctcaaaccatgtgcaacaactggctccgaaacgatcgtttcctcaagaactttgctgctcaaatctgggtgtgcccctgtactctggaacatgctttgctggacaaaggtcgtttcctaccagatctcgattgtgacaaagacaccaacccaacttgtaggtatcactggggaggtgtacattgcgtcaggagtggagcgccaagtgctgaaggatcaggtcaacagtgctgctacgataagaacggctttctcatgctttcatacgatcaaatgtggggatctagacctcacaggtcacacgactttggatttacgccttacaacgaagctaacaaggtaccatcgctgtcccattggttccacgatatgattccattctaccaatgttgttcatggcaagaagaacaagctgttgggtgtgagactttcagattcgaacgtcgtccatcccaagactgtgtcgcgtaccaatctccaggagttgcaggcatattcggagaccctcacatcgtcactttcgatgacctccagtacacatttaacggcaaaggtgaatacgtattggtgagaacagacgaccctcaattgaaactggacgtgcaaggtagattcgagcaggtcgcgagaaacattcatggcgtagtcaatgcaacccatcttacttctatcgtagctgcatctaacaactctgttcctattgaagtcaggttacggcctcagcatgcgcaatggcgataccgactggatgtgttcgctgacaataaacgtgtctactttgacaggtctgctttgaggattcagtacttcccaggtgtcactgtataccagcctatgtacatcctcaaccagtcagaaatcgtgatcatgtttgcatcaggcgctggagttgaagtggtggagaacaagggcttcatgactgccagggtttacctaccctggacgtatatgaacaaaactcgaggtttattcggtaactggtcgctagatgtaaacgatgacttcacgcgccccgacggaatagtgcaacccattgatctcaacaacttccaatcggcacacagagatttcgcgcaacactggcaactaactgatcgggagcaacgaaatataggcgtagcgttattcacaagagaatacggtcgaactgcggcgtactttaacgacaacaccttcataccgaacttcatacgagagccggcggacttcttgcctactaaccggtcgcaggatgtggcaagagcgatagacatctgccaggactcgtaccagtgtcgatacgacttcggcatgaccctgaatagggacatggcggagtttacaaagaattatctgtcttcgattacgaatataaaagaaacgaatgcacgaagagtgatcagttgcggcattttggaaactcctcggtttgggcgaaagagtaacttcttcttcactcctggtactcgggtgaacttcgagtgtaaccaggacttcattctggtcggagacaagcgcagggtgtgcgaggacaacggccggtggaacctccccgactatgggtacactgagtgcttacgtaaccaagaattctctcagcgcgccctatttctgacgtggggtgtgatagtggcaatcattctacctttggccctcttgctctgcctctcgtggttctggtgctggcacaagccgcgctccgagggcaaggagggattcagcttccaggactacccgcgatccaagtccgcttcacggctcaaccttcgatcagcttcaatgggaaatataaccgacactgcgaaatcctccactttacgcagtcaagacacagacaccaaacccaaattacccgaaactcccacggaggaaactcccatgaaaccaagcatgcggtccgctccgctaccccctgaacctcaggatggtgacagctcagggcttgggtatgccgactctagtaaaagcgactccggcaaatcagacaagtcttccattccgaaaaaacgtcgctacgataaaacttaccgaaccaacgaacccttaccaaatttacctgatgtagagttccctgaaaagctctgggatctctctgaagaggacctgctttcgcttacctctccatccgaatctgagtctaatcgtgactctactttgacccgtccggccaaagacattgaatacgtgagcaaacctcgccagactgggcgtcatgctcttgccagtgattcgggctactccacaaaagatggttctgaagatccctatggtaacaaatatgggggcacttacagtcccataccttctccaacttattctgagatttactctcctgccgtgagtccgacttctgacatcagtccgaggagcacattcaataacccgggtctaccagaggcccccaaaagtgcccctgctgatgagataaaaacgttcactcttcctccagtccgtggaaaatcagtagagactttaatagatcctccagttgctgaaatgccgaccttcagcagtcgctctacaatggtttaatcctagcctttgaagctagatagtttctattacttaggactttgtagttctgatcagttaccaccgtaaataaaacgtagactgtacttaatataatcactgcccttgaaatgacaagccgtgggagttttgcggggctcaatctatagtattacttaagtattgattttagatgcacaattatattttttgcacatatttagaagttagattatagattttttgtatttccc

SEQ ID NO: 84 - DNA, 4330 bp, Phyllotreta cruciferae
gtttgaaatcgctcgatgtaaattaaaaaataatattttacacatttatttataaaggatatctaagatctgcaactacatgttcgattacaagtataaaatcttattcctaactaattctaattaaaaaataatttttaacacatttatttataaaccaggatatctacgatctgcaactacatgttcgattacacagtataaaatcttattcctaactaattctacgatactaactaacatttttgtctgcgaattaaatggtttttgtctgtcggtttgcctgaattacgcgaatgaaaactatttctaaatatttcgacacttattgaattattttaatttggctagcgctgcaattccattattctcatcgaataatgtctgcttgctttggaatgtagtaatttagggcctaataaatatttgtttctttagctcccgtgctggtcacgttactatgattatcttcctcctcagaattgaactcttcctcgtgttcggtcaatttcatcgtcgccgcctcctgcgctttcctagattgttctctgtattgttcttgctccagcctgttctcctcccgttctttctgtttcttctttaagaacctgtacgccaaatacacgaatagcaggatgagtggtatgagcactaacattattatcccggacgtgattcctgcttgtcgttgggaatattcctgctgacgcaaacaatatgtgtatccataatccggtatgttccattttccatcacccccgcactctcttctaggatcaccgacgagcacgaaatcttgtatgcattcgtaggtcaccttcgtgccgggcacgaacaggaacgtactcttcctgccgaaccgcggcgtttccagcacgccgcaggacgtgatcttctccctggtggtttgcttcagctcgtaaatggtagccttgtagttctgcgtgaagtgcgccaggtccctgttcagcgtcatggcgtagtcgtagtagcactcgtacatcttggtcgagcacacgtcgtacgtgcgctgtatttgctcggacctgttgggcggcaggatgtcctcgggcagcatcaggaactgcggcttgaaggtcttgttgttgtaggtggccgacgtgcggccgaattccctgtagaacagcgactggcctttgtgcgcgtcgcctacgtcgtccagcatccattttgaaccgaaatcgttgtacactctctccatgtcgttgatgttgctcaccacggccgccttggatccgtccggcagcgtgaagtcgtcttctttgttgaagctccaattgccgaacagccctaccgttttgttaataaacgaccaaggtaagtacaccctggcgctcatgtacccttgattgtccagcacttcgacgccggctccattgtcgaacattattagtacttcagattgattcagaatgtaggttggtgtataaacagtgaccccttggaaatgttggaatttcaacgaaggcctatcaaagaacaatctcctgttctcggctataacatctaatctatacctccacctggcttccaacggtcttcttctcacttcaataggaatggtcttgtttcctttggctacgattgccgtaagttgcgtggctctgacctctccataagcattgatgtccatttgctcgaatctagcctgtatttccaaattatcgtccaccttcaaggatttcaccaaggcgtactctcctttgccgttgaacgtgtactctacgtcatcgaaggtgacaatgtgcggatcaccgtacactcctgccacagccggcgcttgataggcgacgcagtcctgcgaaggccttctttcgaatcgcagcgtttcgcagcccacggcttgctcttcctgccaaagacagcagaggtacttgggtacgatgtcgttgaaccattgggatagtgtggggaccttggtggcttcgttgtaaggcatttttccaaggttgtggcatcgacgggggctggaaccccattgttgatcgtaggacagcattaggtagccgtttttgtcgtagcaacattgttgctcggagccttccattgtgggagcaccggttttgacgcaatgcaaagcttggttgttgtaataacaaacgggattcgaatctttgtcgcaatcaaagtctggcatgaatttccccttatcagccagagcctgttggaccgtgcaaggacattggggtaattcgtgagcgaagtttttcagatatctatcggttctgagccaatcgtcgcagagttgtttgggccaatttttgccgtattggttctcccattggaattggaaataccaacctagaggaatcggcctgctccatattatcggtgtgatgtcgatgttttggttgttggtagagtaaatgttgacctttatcgattcggtcaaatttatttggaggaatccaaacttaatgtcggtgaggaactggttgattttgcctctgtactgcgacggtacgatcgtgtactccccggtgttttggacattatcggctatttctgtgatgtaaacgaacgtgggcctcatggtggtttccctgtaaccccacaaggaaattctgatgttggcattttcgttggtggtcaagttctgcttctgccacgtgatgcgaatctcgctgggcgacttctcgtggtacttcatgtctttgaagaagatcttctgcgaggcactggccggcgattcgacgtagtattttcccttccatttgaaggcgtccgaattgatggcgatttgcagcctcacccaaccctcggagtaaaggcgaggttggatgcaaatagcacgattcctgtccaccacgaatccaacaacttcatcgtacacgtcgaatttacatctgatcctgtcgttgggttggaagcaaggacctgtgatgttcacgaccgttcctcccagcatattgccgctctccggagcaaattgaagaggcaagttagctccatcgatatctttgttacaagaacccagcaggatgttctcgtcgattcggaaaatgtgcctgccgggtttgttgttaccccagccggtggatgttaagtcccgaatgaccgatgcttgactgtagggagcgtactggaagcttctggtgccgttaccggcgttgaatccaacgaatgccggcgtaccgccttcgccttttctagtgtctcctccggcttcggtgtgactggtccagtccaaatttaggtagttgaacatggcgtaggtgaatacctcgtcagtggcgaggaccatttggaatgtgttggtctgataaactgcgttggcgtatcctccattgaaggatacgttcttccacgttacgattatggcgtgtttgggattgaaggtctccgaaccgataactccttctcttatgtcccatttcaacctttcccttatttccactcccatcctgtcttgcctgtccctgaggtctctttccattctgaagtacacacctggtttccttttgtccgagtcttcgtcgcgaagattaccgattttgcatttactgtagaaaataccaataaacgaaggatcattctccttaggccagtcctttacaggaaaaactaatgggtaatccaagttgggaggtggatcgctgaattccaagtaaccattaatggacactctcgtgtaattgtacctgaatccgaagaaaggcaattggaagttcagattcttgtggacctggggcgtggaggattggatttccttctgatagtccccctcgttatccacgttgccccctttgtcgaagaacggatacataaagtttttcctaatctcggctaacctagtttcggtaatggagtaaggcaagcctccacccctttggtccgtgtcctggggggctgtatctgatgtcatggggtcataattggccgcataataagtaacatatttcccggatttagtttttttctggtaagtccagttggctatggaattgttagtatcaattggttcgctatcgatatttgttaatacttctacgggagcgtcgtaattaatgttagtagtaacactcccgccgttttcttcgacgggaatgggcttggaatcgaccccttcgtcgcctcctgacgcacccacgtcgatgctctccacgtctcccgtgttatcctggcccgcggcgtccggcgcgacgccctcttgagcccccacgaccaccgcactgattattaaaactagtataaagttcattttcgctgaaatttcgccctaactccgggcgttgttcgattcaatcgcattaaacgctacttttgcactaggtggcaaaggacctgctgggataagagtatcgtcgcg

SEQ ID NO: 85 - DNA, 4302 bp, Phyllotreta striolata
gtcgtaatcacgacgatactcttatcctagcaggtcctttacaacccagtgcaaaagtagcgtttaatgcgattgaatcgaccaacgcccggcgatagggcggaattttagcgaaaatgcatgtgaaaatgaactttctgctggtgttaataatcagtgcggtggccgtcggggctcaagaggatgccgtagcgcccgatgcagcgagccaggataatacggaagacgtggaaagcatcgacgtgggtgcagcaagaagcgacgtcggcgacgccgatcccaagcccgttcccgtcgaggaaaacaacggcggggcggttacgaataacctcaattacgacgctcccgtagaacaacgctcccgtaaattaacaaatttcgatagcgaaataattgatactaacagttccaaacccaattggactttccagaaaaaaactaaatctggaaaatacgttacttattatggggccaattatgaccccatgacatcagccaccgcccctccggacaccgatcaaagaggtggaggcatcccttacctcattaccgaaaccaggttagcggacattaggaaacactttatgtatccgttcttcgataaagggggcaacggggacaacgagggggattatcagaaggaaatccaatcctctacgccccaagtccacaagaatatgaacttccaattgcctttcttcggattcaggtataattacacgagagtctccatcaacggttatttggaattcagcgatccacctccgaattacgattacccattagtttttccggttaaagactggcctaaacgaaacgatccagcttttattggtattttcttcagtaaatgcaaaatcggtaaccttcgcgacgaagacgccgacaaacgtaaaccaggtgtatacttccgaatggaaagagacctcagggacagacaagacagaatgggagtggaaataagggaaagattgaaatgggacatcagagagggagtaatcggttcggaaaccttcaatcccaaacacgccataatcgtcacatggaaaaacgtatcgttcaatggaggattcggccccgcagtttttcaaaccaatacattccaaatggtcctcgctactgacgaagtattcacgtacgcaatattcaactacctaaacttggaatggaccagtcacacggaagccggtggagacaccagaaacggggaaggcggtgtaccggcattcgttggattcaacgccggtaacggtacaagaagctaccagtacatgccctacagtcaagcatccgtcattcgagatttgacagcgaccggttggggtaacggcaaaccgggaagacacattttccgaatcgacgagaacatcctgctaggttcttgtaacaaagatatcgatggggctaatttaccccttcaatttgcgccggagagcggtaatatgttgggaggaaccgtcgtcaacatcacaggtccctgtttcaatcccaacgataggatcaggtgtaaattcgacgtgttcgatgaagttgtcggatacgtcgtcgacaggaacagagccatttgcattcaacctcctctttacacagaaggttgggtcagactgcaaatcgccgtcaattcggaagcgttcaaatggaagggaaaatactacgtcgaatcgcccgccaccgcctcccagaagatcttcttcaaagacatgaagtaccacgagaagtcgcccagcgagtttcgcatcacgtggcagaagcagaatttgaccaccaacgaaaatgctaacatcagaatctccttgtggggctacagggaaactaccatgagacctacgttcgtttacataactgaaatagccagtacccagaacgccggggagttcctgatctcgccgtcgcagtacaggggcaaagtcaaccaattcttaagtgacatcaagtttggattcttgcaaataaatttgactgaatcggtaaaggtcaacatttactcgacgaccaatcaaaacgtcgacattacaccggttatttggagccggccgattccgcttgcttggtatttccaattccaatgggagagtcaatacgggaagaactggcccaaacacctctgcgacgattggctcagaacggacagatatctgaagaatttcgcacacgaattaccccaatgcccttgcacagtccaacaggctttggcggacaagggaaaattcatgcccgactttgattgcgacaaagattcgaatcctgtttgttattacaataaccaagctttgcattgcgtcaaaactggtgcacccacaatggaaggttccgagcaacaatgttgctacgacaaaaacggttacctgatgctatcctacgatcaacaatggggttcgagtcctcgtcgatgccacaacttgggaaaaatgccttacaatgaagccaccaaagtccccacgctgtctcaatggttcaacgacatagtgcccaaatacctctgttgtctctggcaggaagagcaagccgtgggctgcgaaaccctgcgattcgaacgaaggccttcgcaggattgcgtcgcctatcaagcgccaggtgtagcaggagtgtacggtgacccgcacatagtcaccttcgacgatgtcgaatacacattcaacggtaaaggggagtactccttagtgaaatcggtcaaggtggacgataatttggaaatacaggcgagattcgagcaaatgagccccaatgcttatggagaggtgagagcgacgcaactaacggcgatcgtggctaaaggaaacaagactattcccattgaagttagaagtagaccgatggaatccagatggaggtatagattagatgttatagccgagaacaggaggttgtactttgatagaccttcgttgaaattccaacatttccaaggcgtgactgtttatacaccaacctacattctaaatcaatctgaagtactgataatgttcgacaacggcgccggtgttgaagttctggacaatcaagggtacatgagcgccagagtatacttaccttggtcgtttataaataaaacggttggattgttcggaaattggagtttcaacaaagaagacgacttcaccctgccggacggttccaaggcggccgtcgtgtcaaacatcaacgacatggagagagtgtacaacgacttcggttcgaaatggatgctcgacgacgtcggcgacgagcgcaaaggccagtccttgttctacagggaattcggccgcacctcggccacctacaacaacaagaccttcaggccgcagttcctgatgctgcccgaagacatcctaccgccgaacagatcggagcaaatacagcgcacctacgaggtgtgctccacgaagatgtacgaatgttactacgactacgccatgacgctcaacagagatttagcgcactacacgcaaaactacaaggcgaccatttacgaactgaagcaaaccaccagggagaagatcacgtcctgcggcgtgctggaaacgcccaggttcggcaggaagagcactttcttattcgtacccggcacgaaggtgacttacgaatgtatacaagatttcgtgctagtcggtgatcctagaagagagtgcggggctgatggaaaatggaatattccagaatatggatacacgtattgtttacgtcagcaggaatattcccaacgacaagcaggaatcacgtccgggataataatgttagtgctcataccgctcatcctgctgttcgtgtatttggcgtacaggttcttgaagaagaaacagaaagaacgggatgaaagcaggatgcagcaggaccaatacagagagcaatctaggaaagctcaagaggcagcgacgaggaaattaaccgagcacgaggaggaatacaattctgaagaggaagataataacagtaacgtgaccagtacgggagctaaagaaacaaatatttactaggcttttaaataatacgttcagagccggattttcttaagtcttaggatcaagatgtagataggtaaaagagccttttcatctaaatccagcactggctacatttcaaaccaaacagggagcagtaatcatcttgtagaaatttcaacaagtataatggaagtttcgaaatatttaggaatagttttcattcgcgtaattcacgtaaaaacccgacaaaaatcattcaattcccagacaagtgttagttattattgtagaattagttagaaataagattttatacttgtaatcaataatcaaacatgtagttttagctcatatacatccttgtttaaaataaatacaataa

SEQ ID NO: 86 - DNA, 3847 bp, Megacopta cribraria
cgaccagtcgataacgatgcaggtggctgtgctgcttttgtgcttttgtttcctctcgagcgccaaagaaccgctcagagtcagtgagaacttgttaaatgcttacagatacaggaactatggcagagtcttgggatatggcaacaatactcgtttttatgacgacgaggacgaaaggatggttccttcagacgaccgtaataacggaaattatgtgttgaccgaagacagattgaggaaaataaggtcaaaatttttgtattggtttttcgataaagatcctaaaggaggaactggagatttacaaagagacattactccttcgatcacacaaatccataaaaatcttaatttccaactacctttttttggatatcgctttaattatactagagtgtctttgaatggctatttggaatttagcgacccaccagagcaatattcttatccgctaagttttccagttgtggattggccacataaaaacgacccttcgttcattggaatattctacagtaagtgtagagtgggacaaatgggggaaaatgatgttgatcagagagagcctggcgtttattttagaatggagagagatttaatgtcaagaacagacaaatttggcgtggaggtgagagaaaggttaatgtgggacattagagaaagcgtcgtcggttcggacaccttcattccaaaacatgctgtaattgtcacgtggaagaatgtttcatttgctggtggtattgagaactcagtgagaacgacaaataccttccaattagtcctagcaacggatgaagtatttacctatgcaatgttcaactacgcacaaatcaaatggacaacgcacactgaagccagaggagacacagttggaggagaaggtggtgtaccagcttacattggattcaatgctgggaatggtactcgaagctacgaatacagaccttactcacagcaatctaccatcagagatttagttggccgaggctgggctaatggttttcctggtagacatatttttagaattgatgaaaatattcttcctgggacttgtaacaaggatattgctggggctagtttaccacttacatttgcaccagaaagtggaaacatgctgggtgggacaatcgtcaacataaccggtccctgctttaccccacaaatgaagattatttgcaggttcgatatggaagcagtttatggtacagtcattgatactaatagagcaatatgtgtgcagccttacgttatggcggaaggatacatactttttgatattgccgttgatgatggaaaatatgtgtggaaaggacaatattttattgaaacgccggctacagctgcagaaaaaatatcctttgcagatgacaatgttcatcaaaaatacccaccagaaatcagaataacttggaatcagtacaatttaacaacaaatttagcagctccagtccaaatatctatctggggatacagagaaaaatcaatacgtccacaattaacttacatagatttaatagacgctggagttccaaataatggaatgtatacaataattccatcatcttttaagctacgtgataaccgtatgttcacagacataaattttggatttatacaaattaatttaacaaatcccactaattataatgggctcaaagtaaccccggttatctggagcaaacccatacctttaggttggtattttgcaccacaatgggaaagacaatatggcgaaaattggcctgagcaaaaatgtaacatttggacaatgaatgatcgatatttgaagaattttcctgcagaattgagtatgtgcccgtgcaagttggaacatgctttggcagacaaaggaaggtttatgccggattttgattgtgacatggatgccaatcctcactgcttctatcataaaggggcaaggcattgtgttaaaactggatctcctaggcaagaaggctctgaacaacaatgctgctacgataagaataattatttaatgctttcctatgatcagcagtggggatcgtcaccaaaaaggtctagtaacttgggaattctaccatggaatgaaccaaacaaggtcccaaccctgtctcaatggtaccacgatatagcacctttctatcactgttgtctatggcagtatgaacaagcagtaggatgtgaaacgttcagatttgaaaggagaccctctcaagattgtgtgggttaccaatcaccctacgttgcgaccgttttcggagacccacactttattacttttgatggtttagagtatacattcaatggcaaaggtgagtttgtattactgcacaccagtactgacaagttcaaattagatgtacaagcgcgtttcgagcaagtggggaaaaatatttatggtgatgttgcagccacccaactgacatccctagcagctagagacaatacatcaagtatcatagaagtaagaatgaggccaaaagaggcccaatggagatacagacttgatgtctttgcaaacaagagaagaatatattttgatagaccttcactccgtgtacaacattttacaggtgtcactgtttatcagccatcatatatactcaatcaatcggaaatagtcatcatgttccaatctggagtaggtgttgaggtagttgaaaataaagggtacatggctgcaagagtttatttgccttgggaatacattaatagaactcggggactcttcggtaattggagtttcgatccaagtaacgatttcactcttccagatggaactgtaatgcccgtagaaaaccttaatgattttgaaagaatacataaggacttcgctattcattggaggctggaagataacgttgaagaaagtaaaggtgagcctctttttgttagagagtatgggagaacatcaagctattatttgaatagaacttttactccagtttggaaacgtacgcctcaagaaataattcctccaaataggactaaagatatacaaactgctttgagtttgtgtggagaatcttaccagtgtcgatatgattatgctgtcacgttaaacagggagatggcattttacacattaaactacttcagtgaatttactcatattagacaaacaaataaagaaagagttctgtcatgtggagtgttagaaacaccaagatttggtcacaaatcaaatttcttctttgtacctgattcaaaggtaatatttgagtgcgatcaaggatttatcctagtcggagatcaacgacgaacatgttcacctcaaggaagatggtcatatgaatatggttacactgaatgtcttcggtatattgaatatgatgctcagcagcttggaagaacgatcctaataattggaggagtattggtgcctttgataatgattataatttgcgcagtcgtataccaaaaggggacagttcacaagtttcctggaagttttcaaaagtagacactgttccttccagtgcttacctactcggcagaccacacgattctcctcaacctaatgctccattcttaaagaatgaaagtagcccagattcaaatagaagtattggattccctggccattctaacatttctcttgataaaggacatggaataaaacctgtgatacatttgccatatgataaaagttattatacccatgaaccattaccaaatagacctcacgtagaatttgaagattatgaggacgatgggcttcaaaaaacaggaaattatacaaattatgaagcccccactcttaaagtgaatgacaacttaaaaagacacaatagcattgaaacagatattttttaatctaatttttaaaaatttctaatttttctattttcttattcatcagtca

(19) United States
(12) Patent Application Publication
(10) Pub. No.: US 2017/0253887 A1
(43) Pub. Date: Sep. 7, 2017

(54) COMPOSITIONS AND METHODS TO CONTROL INSECT PESTS

(71) Applicants: PIONEER HI-BRED INTERNATIONAL, INC., Johnston, IA (US); E I DU PONT DE NEMOURS AND COMPANY, WILMINGTON, DE (US)

(72) Inventors: XU HU, JOHNSTON, IA (US); XIPING NIU, JOHNSTON, IA (US); MEGHAN ONEAL, ANKENY, IA (US); JAMES K. PRESNAIL, ST LOUIS, MO (US); NINA RICHTMAN, JOHNSTON, IA (US); JOE ZHAO, JOHNSTON, IA (US)

(73) Assignees: PIONEER HI-BRED INTERNATIONAL, INC., JOHNSTON, IA (US); E I DU PONT DE NEMOURS AND COMPANY, WILMINGTON, DE (US)

(21) Appl. No.: 15/511,196

(22) PCT Filed: Aug. 31, 2015
(86) PCT No.: PCT/US15/47697

§ 371 Date: Mar. 14, 2017

Related U.S. Application Data
(60) Provisional application No. 62/051,894, filed on Sep. 17, 2014.

(51) Int. Cl. C12N 15/82; C12N 15/113

(52) U.S. Cl. CPC C12N 15/8286; C12N 15/8218; C12N 15/113; C12N 2310/14; C12N 2310/531

(57) ABSTRACT

Methods and compositions are provided which employ a silencing element that, when ingested by a pest, such as a Coleopteran plant pest or a Diabrotica plant pest, decrease the expression of a target sequence in the pest. Disclosed are various target polynucleotides set forth in any one of SEQ ID NOS: disclosed herein, (but not including the forward and reverse primers.) or variants or fragments thereof, or complements thereof, wherein a decrease in expression of one or more of the sequences in the target pest controls the pest (i.e., has insecticidal activity). Plants, plant parts, bacteria and other host cells comprising the silencing elements, variants or fragments thereof, or complements thereof, are also provided.

### CROSS-REFERENCE TO RELATED APPLICATIONS

This application claims priority to U.S. Provisional Application No. 62/051,894, filed Sep. 17, 2014, which is hereby incorporated herein in its entirety by reference.

### FIELD OF THE INVENTION

The Disclosed herein are methods and compositions generally relating to molecular biology and gene silencing to control pests.

### REFERENCE TO A SEQUENCE LISTING SUBMITTED AS A TEXT FILE VIA EFS-WEB

The Sequence Listing submitted Sep. 17, 2014 as a text file named “6496WOPCT_SeqListing.txt,” created on Sep. 17, 2014, and having a size of 193,297 bytes is hereby incorporated by reference pursuant to 37 C.F.R. §1.52(e)(5).

### BACKGROUND OF THE INVENTION

Plant insect pests are a serious problem in agriculture. They destroy millions of acres of staple crops such as corn, soybeans, peas, and cotton. Yearly, these pests cause over $100 billion dollars in crop damage in the U.S. alone. In an ongoing seasonal battle, farmers must apply billions of gallons of synthetic pesticides to combat these pests. Other methods employed in the past delivered insecticidal activity by microorganisms or genes derived from microorganisms expressed in transgenic plants. For example, certain species of microorganisms of the genus Bacillus are known to possess pesticidal activity against a broad range of insect pests including Lepidoptera, Diptera, Coleoptera, Hemiptera, and others. In fact, microbial pesticides, particularly those obtained from Bacillus strains, have played an important role in agriculture as alternatives to chemical pest control. Agricultural scientists have developed crop plants with enhanced insect resistance by genetically engineering crop plants to produce insecticidal proteins from Bacillus. For example, corn and cotton plants genetically engineered to produce Cry toxins (see, e.g., Aronson (2002) Cell Mol. Life Sci. 59(3):417-425; Schnepf et al. (1998) Microbiol. Mol. Biol. Rev. 62(3):775-806) are now widely used in American agriculture and have provided the farmer with an alternative to traditional insect-control methods. However, these Bt insecticidal proteins only protect plants from a relatively narrow range of pests. Thus, novel insect control compositions remain desirable.

### BRIEF SUMMARY OF THE INVENTION

Methods and compositions are provided which employ a silencing element that, when ingested by a plant insect pest, such as Coleopteran plant pest including a Diabrotica plant pest, is capable of decreasing the expression of a target sequence in the pest. In specific embodiments, the decrease in expression of the target sequence controls the pest and thereby the methods and compositions are capable of limiting damage to a plant. Described herein are various target polynucleotides as set forth in SEQ ID NOs.: 1-86 or variants or fragments thereof, or complements thereof, wherein a decrease in expression of one or more of the sequences in the target pest controls the pest (i.e., has insecticidal activity). Further provided are silencing elements, which when ingested by the pest, decrease the level of expression of one or more of the target polynucleotides. Plants, plant parts, plant cells, bacteria and other host cells comprising the silencing elements or an active variant or fragment thereof are also provided. Also provided are formulations of sprayable silencing agents for topical applications to pest insects or substrates where pest insects may be found.

In an embodiment, a method for controlling a plant insect pest, such as a Coleopteran plant pest or a Diabrotica plant pest, is provided. The method comprises feeding to an insect pest a composition comprising a silencing element, wherein the silencing element, when ingested by the pest, reduces the level of a target sequence in the pest and thereby controls the pest. Further provided are methods to protect a plant from a pest. Such methods comprise introducing into the plant or plant part a silencing element of the invention. When the plant expressing the silencing element is ingested by the pest, the level of the target sequence is decreased and the pest is controlled.

### BRIEF DESCRIPTION OF THE DRAWINGS

FIG. 1 is a representative survival plot for one day old Western Corn Root Worm larvae (“WCRW”) fed a diet comprising RYANR-Frag 1 (SEQ ID NO.: 1; described in Table 1) at 5 ppm in two tests (as indicated in the figure) per assay methods as described herein.

FIG. 2 is a graph showing representative data for the survival of adult WCRW beetles exposed to different dsRNAi treatments. Both RYANR (DV-RYANR-FRAG1, SEQ ID NO.: 1) and GUS dsRNA were tested at 100 ppm/day/insect in 25 μl:75 μl (sample:diet plug ratio).

### DETAILED DESCRIPTION OF THE INVENTION

The disclosures herein will be described more fully hereinafter with reference to the accompanying drawings, in which some, but not all embodiments of the inventions are shown. Indeed, disclosures may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments disclosed herein will come to mind to one skilled in the art to which these inventions pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which this invention belongs. In this specification and in the claims which follow, reference will be made to a number of terms which shall be defined herein.

### I. Overview

Frequently, RNAi discovery methods rely on evaluation of known classes of sensitive genes (transcription factors, housekeeping genes etc.). In contrast, target polynucleotides set forth herein were identified based solely on high throughput screens of all singletons and representatives of all gene clusters from a cDNA library of neonate and/or 3^{rd} instar midgut western corn rootworms. This method provided the advantage of having no built-in bias to genes that are frequently highly conserved across taxa. As a result, many novel targets for RNAi as well as known genes not previously shown to be sensitive to RNAi have been identified.

As such, methods and compositions are provided which employ one or more silencing elements that, when ingested by a plant insect pest, such as a Coleopteran plant pest or a Diabrotica plant pest, is capable of decreasing the expression of a target sequence in the pest. In specific embodiments, the decrease in expression of the target sequence controls the pest and thereby the methods and compositions are capable of limiting damage to a plant or plant part. Described herein are target polynucleotides as set forth in SEQ ID NOS.: 1-86 or variants or fragments thereof, or complements thereof. Silencing elements comprising sequences, complementary sequences, active fragments or variants of these target polynucleotides are provided which, when ingested by or when contacting the pest, decrease the expression of one or more of the target sequences and thereby controls the pest (i.e., has insecticidal activity).

As used herein, by “controlling a pest” or “controls a pest” is intended any affect on a pest that results in limiting the damage that the pest causes. Controlling a pest includes, but is not limited to, killing the pest, inhibiting development of the pest, altering fertility or growth of the pest in such a manner that the pest provides less damage to the plant, or in a manner for decreasing the number of offspring produced, producing less fit pests, producing pests more susceptible to predator attack, or deterring the pests from eating the plant.

Reducing the level of expression of the target polynucleotide or the polypeptide encoded thereby, in the pest results in the suppression, control, and/or killing the invading pest. Reducing the level of expression of the target sequence of the pest will reduce the pest damage by at least about 2% to at least about 6%, at least about 5% to about 50%, at least about 10% to about 60%, at least about 30% to about 70%, at least about 40% to about 80%, or at least about 50% to about 90% or greater. Hence, methods disclosed herein can be utilized to control insect pests, including but not limited to, Coleopteran plant pests or a Diabrotica plant pest.

Assays measuring the control of a pest are commonly known in the art, as are methods to record nodal injury score. See, for example, Oleson et al. (2005) J. Econ. Entomol. 98:1-8. See, for example, the examples below.

Disclosed herein are compositions and methods for protecting plants from a plant insect pest, such as Coleopteran plant pests or Diabrotica plant pests or other plant pest insects, or inducing resistance in a plant to a plant insect pest, such as Coleopteran plant pests or Diabrotica plant pests or other plant pest insects. Plant insect pests include insects selected from the orders Coleoptera, Diptera, Hymenoptera, Lepidoptera, Mallophaga, Homoptera, Hemiptera Orthroptera, Thysanoptera, Dermaptera, Isoptera, Anoplura, Siphonaptera, Trichoptera, etc., particularly Lepidoptera and Coleoptera.

Those skilled in the art will recognize that not all compositions are equally effective against all pests. Disclosed compositions, including the silencing elements disclosed herein, display activity against plant insect pests, which may include economically important agronomic, forest, greenhouse, nursery ornamentals, food and fiber, public and animal health, domestic and commercial structure, household and stored product pests.

As used herein “Coleopteran plant pest” is used to refer to any member of the Coleoptera order. Other plant pests that may be targeted by the methods and compositions of disclosed herein include, but are not limited to, Mexican Bean Beetle (Epilachna varivestis), and Colorado potato beetle (Leptinotarsa decemlineata).

As used herein, the term “Diabrotica plant pest” is used to refer to any member of the Diabrotica genus. Accordingly, the compositions and methods are also useful in protecting plants against any Diabrotica plant pest including, for example, Diabrotica adelpha; Diabrotica amecameca; Diabrotica balteata; Diabrotica barberi; Diabrotica biannularis; Diabrotica cristata; Diabrotica decempunctata; Diabrotica dissimilis; Diabrotica lemniscata; Diabrotica limitata (including, for example, Diabrotica limitata quindecimpuncata); Diabrotica longicornis; Diabrotica nummularis; Diabrotica porracea; Diabrotica scutellata; Diabrotica sexmaculata; Diabrotica speciosa (including, for example, Diabrotica speciosa speciosa); Diabrotica tibialis; Diabrotica undecimpunctata (including, for example, Southern corn rootworm (Diabrotica undecimpunctata), Diabrotica undecimpunctata duodecimnotata; Diabrotica undecimpunctata howardi (spotted cucumber beetle); Diabrotica undecimpunctata undecimpunctata (western spotted cucumber beetle)); Diabrotica virgifera (including, for example, Diabrotica virgifera virgifera (western corn rootworm) and Diabrotica virgifera zeae (Mexican corn rootworm)); Diabrotica viridula; Diabrotica wartensis; Diabrotica sp. JJG335; Diabrotica sp. JJG336; Diabrotica sp. JJG341; Diabrotica sp. JJG356; Diabrotica sp. JJG362; and, Diabrotica sp. JJG365.

In specific embodiments, the Diabrotica plant pest comprises D. virgifera virgifera, D. barberi, D. virgifera zeae, D. speciosa, or D. undecimpunctata howardi.

Larvae of the order Lepidoptera include, but are not limited to, armyworms, cutworms, loopers and heliothines in the family Noctuidae Spodoptera frugiperda JE Smith (fall armyworm); S. exigua Hübner (beet armyworm); S. litura Fabricius (tobacco cutworm, cluster caterpillar); Mamestra configurata Walker (bertha armyworm); M. brassicae Linnaeus (cabbage moth); Agrotis ipsilon Hufnagel (black cutworm); A. orthogonia Morrison (western cutworm); A. subterranea Fabricius (granulate cutworm); Alabama argillacea Hübner (cotton leaf worm); Trichoplusia ni Hübner (cabbage looper); Pseudoplusia includens Walker (soybean looper); Anticarsia gemmatalis Hübner (velvetbean caterpillar); Hypena scabra Fabricius (green cloverworm); Heliothis virescens Fabricius (tobacco budworm); Pseudaletia unipuncta Haworth (armyworm); Athetis mindara Barnes and Mcdunnough (rough skinned cutworm); Euxoa messoria Harris (darksided cutworm); Earias insulana Boisduval (spiny bollworm); E. vittella Fabricius (spotted bollworm); Helicoverpa armigera Hübner (American bollworm); H. zea Boddie (corn earworm or cotton bollworm); Melanchra picta Harris (zebra caterpillar); Egira (Xylomyges) curialis Grote (citrus cutworm); borers, casebearers, webworms, coneworms, and skeletonizers from the family Pyralidae Ostrinia nubilalis Hübner (European corn borer); Amyelois transitella Walker (naval orangeworm); Anagasta kuehniella Zeller (Mediterranean flour moth); Cadra cautella Walker (almond moth); Chilo suppressalis Walker (rice stem borer); C. partellus, (sorghum borer); Corcyra cephalonica Stainton (rice moth); Crambus caliginosellus Clemens (corn root webworm); C. teterrellus Zincken (bluegrass webworm); Cnaphalocrocis medinalis Guenée (rice leaf roller); Desmia funeralis Hübner (grape leaffolder); Diaphania hyalinata Linnaeus (melon worm); D. nitidalis Stoll (pickleworm); Diatraea grandiosella Dyar (southwestern corn borer), D. saccharalis Fabricius (sugarcane borer); Eoreuma loftini Dyar (Mexican rice borer); Ephestia elutella Hübner (tobacco (cacao) moth); Galleria mellonella Linnaeus (greater wax moth); Herpetogramma licarsisalis Walker (sod webworm); Homoeosoma electellum Hulst (sunflower moth); Elasmopalpus lignosellus Zeller (lesser cornstalk borer); Achroia grisella Fabricius (lesser wax moth); Loxostege sticticalis Linnaeus (beet webworm); Orthaga thyrisalis Walker (tea tree web moth); Maruca testulalis Geyer (bean pod borer); Plodia interpunctella Hübner (Indian meal moth); Scirpophaga incertulas Walker (yellow stem borer); Udea rubigalis Guenée (celery leaftier); and leafrollers, budworms, seed worms and fruit worms in the family Tortricidae Acleris gloverana Walsingham (Western blackheaded budworm); A. variana Fernald (Eastern blackheaded budworm); Archips argyrospila Walker (fruit tree leaf roller); A. rosana Linnaeus (European leaf roller); and other Archips species, Adoxophyes orana Fischer von Rosslerstamm (summer fruit tortrix moth); Cochylis hospes Walsingham (banded sunflower moth); Cydia latiferreana Walsingham (filbertworm); C. pomonella Linnaeus (coding moth); Platynota flavedana Clemens (variegated leafroller); P. stultana Walsingham (omnivorous leafroller); Lobesia botrana Denis & Schiffermüller (European grape vine moth); Spilonota ocellana Denis & Schiffermüller (eyespotted bud moth); Endopiza viteana Clemens (grape berry moth); Eupoecilia ambiguella Hübner (vine moth); Bonagota salubricola Meyrick (Brazilian apple leafroller); Grapholita molesta Busck (oriental fruit moth); Suleima helianthana Riley (sunflower bud moth); Argyrotaenia spp.; Choristoneura spp.

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guérin-Méneville (Chinese Oak Tussah Moth); Bombyx mori Linnaeus (Silkworm); Bucculatrix thurberiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hübner (elm spanworm); Erannis tiliaria Harris (linden looper); Euproctis chrysorrhoea Linnaeus (browntail moth); Harrisina americana Guérin-Méneville (grapeleaf skeletonizer); Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Manduca quinquemaculata Haworth (five spotted hawk moth, tomato hornworm); M. sexta Haworth (tomato hornworm, tobacco hornworm); Operophtera brumata Linnaeus (winter moth); Paleacrita vernata Peck (spring cankerworm); Papilio cresphontes Cramer (giant swallowtail orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval and Leconte (Southern cabbageworm); Sabulodes aegrotata Guenée (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tuta absoluta Meyrick (tomato leafminer); Yponomeuta padella Linnaeus (ermine moth); Heliothis subflexa Guenée; Malacosoma spp. and Orgyia spp.

Of interest are larvae and adults of the order Coleoptera including weevils from the families Anthribidae, Bruchidae and Curculionidae (including, but not limited to: Anthonomus grandis Boheman (boll weevil); Lissorhoptrus oryzophilus Kuschel (rice water weevil); Sitophilus granarius Linnaeus (granary weevil); S. oryzae Linnaeus (rice weevil); Hypera punctata Fabricius (clover leaf weevil); Cylindrocopturus adspersus LeConte (sunflower stem weevil); Smicronyx fulvus LeConte (red sunflower seed weevil); S. sordidus LeConte (gray sunflower seed weevil); Sphenophorus maidis Chittenden (maize billbug)); flea beetles, cucumber beetles, rootworms, leaf beetles, potato beetles and leafminers in the family Chrysomelidae (including, but not limited to: Leptinotarsa decemlineata Say (Colorado potato beetle); Diabrotica virgifera virgifera LeConte (western corn rootworm); D. barberi Smith and Lawrence (northern corn rootworm); D. undecimpunctata howardi Barber (southern corn rootworm); Chaetocnema pulicaria Melsheimer (corn flea beetle); Phyllotreta cruciferae Goeze (Crucifer flea beetle); Phyllotreta striolata (stripped flea beetle); Colaspis brunnea Fabricius (grape colaspis); Oulema melanopus Linnaeus (cereal leaf beetle); Zygogramma exclamationis Fabricius (sunflower beetle)); beetles from the family Coccinellidae (including, but not limited to: Epilachna varivestis Mulsant (Mexican bean beetle)); chafers and other beetles from the family Scarabaeidae (including, but not limited to: Popillia japonica Newman (Japanese beetle); Cyclocephala borealis Arrow (northern masked chafer, white grub); C. immaculata Olivier (southern masked chafer, white grub); Rhizotrogus majalis Razoumowsky (European chafer); Phyllophaga crinita Burmeister (white grub); Ligyrus gibbosus De Geer (carrot beetle)); carpet beetles from the family Dermestidae; wireworms from the family Elateridae, Eleodes spp., Melanotus spp.; Conoderus spp.; Limonius spp.; Agriotes spp.; Ctenicera spp.; Aeolus spp.; bark beetles from the family Scolytidae and beetles from the family Tenebrionidae.

Adults and immatures of the order Diptera are of interest, including leafminers Agromyza parvicornis Loew (corn blotch leafminer); midges (including, but not limited to: Contarinia sorghicola Coquillett (sorghum midge); Mayetiola destructor Say (Hessian fly); Sitodiplosis mosellana Géhin (wheat midge); Neolasioptera murtfeldtiana Felt, (sunflower seed midge)); fruit flies (Tephritidae), Oscinella frit Linnaeus (fruit flies); maggots (including, but not limited to: Delia platura Meigen (seedcorn maggot); D. coarctata Fallen (wheat bulb fly) and other Delia spp., Meromyza americana Fitch (wheat stem maggot); Musca domestica Linnaeus (house flies); Fannia canicularis Linnaeus, F. femoralis Stein (lesser house flies); Stomoxys calcitrans Linnaeus (stable flies)); face flies, horn flies, blow flies, Chrysomya spp.; Phormia spp. and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds) and other Brachycera, mosquitoes Aedes spp.; Anopheles spp.; Culex spp.; black flies Prosimulium spp.; Simulium spp.; biting midges, sand flies, sciarids, and other Nematocera.

Included as insects of interest are adults and nymphs of the orders Hemiptera and Homoptera such as, but not limited to, adelgids from the family Adelgidae, plant bugs from the family Miridae, cicadas from the family Cicadidae, leafhoppers, Empoasca spp.; from the family Cicadellidae, planthoppers from the families Cixiidae, Flatidae, Fulgoroidea, Issidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Asterolecanidae, Coccidae, Dactylopiidae, Diaspididae, Eriococcidae Ortheziidae, Phoenicococcidae and Margarodidae, lace bugs from the family Tingidae, stink bugs from the family Pentatomidae, cinch bugs, Blissus spp.; and other seed bugs from the family Lygaeidae, spittlebugs from the family Cercopidae squash bugs from the family Coreidae and red bugs and cotton stainers from the family Pyrrhocoridae.

Agronomically important members from the order Homoptera further include, but are not limited to: Acyrthisiphon pisum Harris (pea aphid); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacorthum solani Kaltenbach (foxglove aphid); Chaetosiphon fragaefolii Cockerell (strawberry aphid); Diuraphis noxia Kurdjumov/Mordvilko (Russian wheat aphid); Dysaphis plantaginea Paaserini (rosy apple aphid); Eriosoma lanigerum Hausmann (woolly apple aphid); Brevicoryne brassicae Linnaeus (cabbage aphid); Hyalopterus pruni Geoffroy (mealy plum aphid); Lipaphis erysimi Kaltenbach (turnip aphid); Metopolophium dirrhodum Walker (cereal aphid); Macrosiphum euphorbiae Thomas (potato aphid); Myzus persicae Sulzer (peach-potato aphid, green peach aphid); Nasonovia ribisnigri Mosley (lettuce aphid); Pemphigus spp. (root aphids and gall aphids); Rhopalosiphum maidis Fitch (corn leaf aphid); R. padi Linnaeus (bird cherry-oat aphid); Schizaphis graminum Rondani (greenbug); Sipha flava Forbes (yellow sugarcane aphid); Sitobion avenae Fabricius (English grain aphid); Therioaphis maculata Buckton (spotted alfalfa aphid); Toxoptera aurantii Boyer de Fonscolombe (black citrus aphid) and T. citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly); B. argentifolii Bellows & Perring (silverleaf whitefly); Dialeurodes citri Ashmead (citrus whitefly); Trialeurodes abutiloneus (bandedwinged whitefly) and T. vaporariorum Westwood (greenhouse whitefly); Empoasca fabae Harris (potato leafhopper); Laodelphax striatellus Fallen (smaller brown planthopper); Macrolestes quadrilineatus Forbes (aster leafhopper); Nephotettix cinticeps Uhler (green leafhopper); N. nigropictus Stil (rice leafhopper); Nilaparvata lugens Stil (brown planthopper); Peregrinus maidis Ashmead (corn planthopper); Sogatella furcifera Horvath (white-backed planthopper); Sogatodes orizicola Muir (rice delphacid); Typhlocyba pomaria McAtee (white apple leafhopper); Erythroneoura spp. (grape leafhoppers); Magicicada septendecim Linnaeus (periodical cicada); Icerya purchasi Maskell (cottony cushion scale); Quadraspidiotus perniciosus Comstock (San Jose scale); Planococcus citri Risso (citrus mealybug); Pseudococcus spp. (other mealybug complex); Cacopsylla pyricola Foerster (pear psylla); Trioza diospyri Ashmead (persimmon psylla).

Agronomically important species of interest from the order Hemiptera include, but are not limited to: Acrosternum hilare Say (green stink bug); Anasa tristis De Geer (squash bug); Blissus leucopterus leucopterus Say (chinch bug); Corythuca gossypii Fabricius (cotton lace bug); Cyrtopeltis modesta Distant (tomato bug); Dysdercus suturellus Herrich-Schiffer (cotton stainer); Euschistus servus Say (brown stink bug); E. variolarius Palisot de Beauvois (one-spotted stink bug); Graptostethus spp. (complex of seed bugs); Leptoglossus corculus Say (leaf-footed pine seed bug); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Lygocoris pabulinus Linnaeus (common green capsid); Nezara viridula Linnaeus (southern green stink bug); Oebalus pugnax Fabricius (rice stink bug); Oncopeltus fasciatus Dallas (large milkweed bug); Pseudatomoscelis seriatus Reuter (cotton fleahopper).

Furthermore, embodiments may be effective against Hemiptera such, Calocoris norvegicus Gmelin (strawberry bug); Orthops campestris Linnaeus; Plesiocoris rugicollis Fallen (apple capsid); Cyrtopeltis modestus Distant (tomato bug); Cyrtopeltis notatus Distant (suckfly); Spanagonicus albofasciatus Reuter (whitemarked fleahopper); Diaphnocoris chlorionis Say (honeylocust plant bug); Labopidicola allii Knight (onion plant bug); Pseudatomoscelis seriatus Reuter (cotton fleahopper); Adelphocoris rapidus Say (rapid plant bug); Poecilocapsus lineatus Fabricius (four-lined plant bug); Nysius ericae Schilling (false chinch bug); Nysius raphanus Howard (false chinch bug); Nezara viridula Linnaeus (Southern green stink bug); Eurygaster spp.; Coreidae spp.; Pyrrhocoridae spp.; Tinidae spp.; Blostomatidae spp.; Reduviidae spp. and Cimicidae spp.

Also included are adults and larvae of the order Acari (mites) such as Aceria tosichella Keifer (wheat curl mite); Petrobia latens Muller (brown wheat mite); spider mites and red mites in the family Tetranychidae, Panonychus ulmi Koch (European red mite); Tetranychus urticae Koch (two spotted spider mite); (T. mcdanieli McGregor (McDaniel mite); T. cinnabarinus Boisduval (carmine spider mite); T. turkestani Ugarov & Nikolski (strawberry spider mite); flat mites in the family Tenuipalpidae, Brevipalpus lewisi McGregor (citrus flat mite); rust and bud mites in the family Eriophyidae and other foliar feeding mites and mites important in human and animal health, i.e., dust mites in the family Epidermoptidae, follicle mites in the family Demodicidae, grain mites in the family Glycyphagidae, ticks in the order Ixodidae. Ixodes scapularis Say (deer tick); I. holocyclus Neumann (Australian paralysis tick); Dermacentor variabilis Say (American dog tick); Amblyomma americanum Linnaeus (lone star tick) and scab and itch mites in the families Psoroptidae, Pyemotidae and Sarcoptidae.

Insect pests of the order Thysanura are of interest, such as Lepisma saccharina Linnaeus (silverfish); Thermobia domestica Packard (firebrat).

Insect pest of interest include the superfamily of stink bugs and other related insects including but not limited to species belonging to the family Pentatomidae (Nezara viridula, Halyomorpha halys, Piezodorus guildini, Euschistus servus, Acrostemum hilare, Euschistus heros, Euschistus tristigmus, Acrosternum hilare, Dichelops furcatus, Dichelops melacanthus, and Bagrada hilaris (Bagrada Bug)), the family Plataspidae (Megacopta cribraria—Bean plataspid) and the family Cydnidae (Scaptocoris castanea—Root stink bug) and Lepidoptera species including but not limited to: diamond-back moth, e.g., Helicoverpa zea Boddie; soybean looper, e.g., Pseudoplusia includens Walker and velvet bean caterpillar e.g., Anticarsia gemmatalis Hübner.

### II. Target Sequences

As used herein, a “target sequence” or “target polynucleotide” comprises any sequence in the pest that one desires to reduce the level of expression thereof. In specific embodiments, decreasing the level of the target sequence in the pest controls the pest. For instance, the target sequence may be essential for growth and development. While the target sequence can be expressed in any tissue of the pest, in specific embodiments, the sequences targeted for suppression in the pest are expressed in cells of the gut tissue of the pest, cells in the midgut of the pest, and cells lining the gut lumen or the midgut. Such target sequences can be involved in, for example, gut cell metabolism, growth or differentiation. Non-limiting examples of target sequences of the invention include a polynucleotide set forth in SEQ ID NOS.: 1-86 or variants or fragments thereof, or complements thereof. As exemplified elsewhere herein, decreasing the level of expression of one or more of these target sequences in a Coleopteran plant pest or a Diabrotica plant pest controls the pest.

### III. Silencing Elements

By “silencing element” is intended a polynucleotide which when contacted by or ingested by a pest, is capable of reducing or eliminating the level or expression of a target polynucleotide or the polypeptide encoded thereby. In one embodiment, the silencing element employed can reduce or eliminate the expression level of the target sequence by influencing the level of the target RNA transcript or, alternatively, by influencing translation and thereby affecting the level of the encoded polypeptide. Methods to assay for functional silencing elements that are capable of reducing or eliminating the level of a sequence of interest are disclosed elsewhere herein. A single polynucleotide employed in the methods of the invention can comprise one or more silencing elements to the same or different target polynucleotides. The silencing element can be produced in vivo (i.e., in a host cell such as a plant or microorganism) or in vitro. It is to be understood that “silencing element,” as used herein, is intended to comprise polynucleotides such as RNA constructs, DNA constructs that encode the RNA constructs, and/or expression constructs comprising the DNA constructs.

In specific embodiments, a silencing element may comprise a chimeric construction molecule comprising two or more sequences as disclosed herein. For example, the chimeric construction may be a hairpin or dsRNA as disclosed herein. A chimera may comprise two or more sequences as disclosed herein. In one embodiment, a chimera contemplates two complementary sequences set forth herein having some degree of mismatch between the complementary sequences such that the two sequences are not perfect complements of one another. Providing at least two different sequences in a single silencing element may allow for targeting multiple genes using one silencing element and/or for example, one expression cassette. Targeting multiple genes may allow for slowing or reducing the possibility of resistance by the pest, and providing the multiple targeting ability in one expressed molecule may reduce the expression burden of the transformed plant or plant product, or provide topical treatments that are capable of targeting multiple hosts with one application.

In specific embodiments, the target sequence is not endogenous to the plant. In other embodiments, while the silencing element controls pests, preferably the silencing element has no effect on the normal plant or plant part.

As discussed in further detail below, silencing elements can include, but are not limited to, a sense suppression element, an antisense suppression element, a double stranded RNA, a siRNA, an amiRNA, a miRNA, or a hairpin suppression element. Silencing elements disclosed herein may comprise a chimera where two or more sequences disclosed herein or active fragments or variants, or complements thereof, are found in the same RNA molecule. Further, a sequence disclosed herein, or active fragment or variant, or complement thereof, may be present as more than one copy in a DNA construct, silencing element, DNA molecule or RNA molecule. In a hairpin or dsRNA molecule, the location of a sense or antisense sequence in the molecule, for example, in which sequence is transcribed first or is located on a particular terminus of the RNA molecule, is not limiting to the invention, and the invention is not to be limited by disclosures herein of a particular location for such a sequence. Non-limiting examples of silencing elements that can be employed to decrease expression of these target sequences comprise fragments or variants of the sense or antisense sequence or consists of the sense or antisense sequence of a sequence set forth in SEQ ID NOS.: 1-86 or variants or fragments thereof, or complements thereof. The silencing element can further comprise additional sequences that advantageously effect transcription and/or the stability of a resulting transcript. For example, the silencing elements can comprise at least one thymine residue at the 3′ end. This can aid in stabilization. Thus, the silencing elements can have at least 1, 2, 3, 4, 5, 6, 7, 8, 9, 10 or more thymine residues at the 3′ end. As discussed in further detail below, enhancer suppressor elements can also be employed in conjunction with the silencing elements disclosed herein.

By “reduces” or “reducing” the expression level of a polynucleotide or a polypeptide encoded thereby is intended to mean, the polynucleotide or polypeptide level of the target sequence is statistically lower than the polynucleotide level or polypeptide level of the same target sequence in an appropriate control pest which is not exposed to (i.e., has not ingested or come into contact with) the silencing element. In particular embodiments of the invention, reducing the polynucleotide level and/or the polypeptide level of the target sequence in a pest according to the invention results in less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control pest. Methods to assay for the level of the RNA transcript, the level of the encoded polypeptide, or the activity of the polynucleotide or polypeptide are discussed elsewhere herein.

i. Sense Suppression Elements

As used herein, a “sense suppression element” comprises a polynucleotide designed to express an RNA molecule corresponding to at least a part of a target messenger RNA in the “sense” orientation. Expression of the RNA molecule comprising the sense suppression element reduces or eliminates the level of the target polynucleotide or the polypeptide encoded thereby. The polynucleotide comprising the sense suppression element may correspond to all or part of the sequence of the target polynucleotide, all or part of the 5′ and/or 3′ untranslated region of the target polynucleotide, all or part of the coding sequence of the target polynucleotide, or all or part of both the coding sequence and the untranslated regions of the target polynucleotide.

Typically, a sense suppression element has substantial sequence identity to the target polynucleotide, typically greater than about 65% sequence identity, greater than about 85% sequence identity, about 90%, 91%, 92%, 93%, 94%, 95%, 96%, 97%, 98% or 99% sequence identity. See, U.S. Pat. Nos. 5,283,184 and 5,034,323; herein incorporated by reference. The sense suppression element can be any length so long as it allows for the suppression of the targeted sequence. The sense suppression element can be, for example, 15, 16, 17, 18, 19, 20, 22, 25, 30, 50, 100, 150, 200, 250, 300, 350, 400, 450, 500, 600, 700, 900, 1000, 1100, 1200, 1300 nucleotides or longer of the target polynucleotides set forth in any of SEQ ID NOS.: 1-86 and variants and fragments thereof, and complements thereof. In other embodiments, the sense suppression element can be, for example, about 15-25, 19-35, 19-50, 25-100, 100-150, 150-200, 200-250, 250-300, 300-350, 350-400, 450-500, 500-550, 550-600, 600-650, 650-700, 700-750, 750-800, 800-850, 850-900, 900-950, 950-1000, 1000-1050, 1050-1100, 1100-1200, 1200-1300, 1300-1400, 1400-1500, 1500-1600, 1600-1700, 1700-1800 nucleotides or longer of the target polynucleotides set forth in any of SEQ ID NOS.: 1-86 and variants and fragments thereof, and complements thereof.

ii. Antisense Suppression Elements

As used herein, an “antisense suppression element” comprises a polynucleotide which is designed to express an RNA molecule complementary to all or part of a target messenger RNA. Expression of the antisense RNA suppression element reduces or eliminates the level of the target polynucleotide. The polynucleotide for use in antisense suppression may correspond to all or part of the complement of the sequence encoding the target polynucleotide, all or part of the complement of the 5′ and/or 3′ untranslated region of the target polynucleotide, all or part of the complement of the coding sequence of the target polynucleotide, or all or part of the complement of both the coding sequence and the untranslated regions of the target polynucleotide. In addition, the antisense suppression element may be fully complementary (i.e., 100% identical to the complement of the target sequence) or partially complementary (i.e., less than 100% identical to the complement of the target sequence) to the target polynucleotide. In specific embodiments, the antisense suppression element comprises at least 85%, 90%, 91%, 92%, 93%, 94%, 95%, 96%, 97%, 98%, or 99% sequence complementarity to the target polynucleotide. Antisense suppression may be used to inhibit the expression of multiple proteins in the same plant. See, for example, U.S. Pat. No. 5,942,657. Furthermore, the antisense suppression element can be complementary to a portion of the target polynucleotide. Generally, sequences of at least 15, 16, 17, 18, 19, 20, 22, 25, 50, 100, 200, 300, 400, 450 nucleotides or greater of the sequence set forth in any of SEQ ID NOS.: 1-86 and variants and fragments thereof, and complements thereof may be used. Methods for using antisense suppression to inhibit the expression of endogenous genes in plants are described, for example, in Liu et al (2002) Plant Physiol. 129:1732-1743 and U.S. Pat. No. 5,942,657, which is herein incorporated by reference.

iii. Double Stranded RNA Suppression Element

A “double stranded RNA silencing element” or “dsRNA”, which may also be referred to as “dsRNA construct”, comprises at least one transcript that is capable of forming a dsRNA either before or after ingestion by a pest. Thus, a “dsRNA silencing element” includes a dsRNA, a transcript or polyribonucleotide capable of forming a dsRNA or more than one transcript or polyribonucleotide capable of forming a dsRNA. “Double stranded RNA” or “dsRNA” refers to a polyribonucleotide structure formed either by a single self-complementary RNA molecule or a polyribonucleotide structure formed by the expression of at least two distinct RNA strands. The dsRNA molecule(s) employed in the methods and compositions of the invention mediate the reduction of expression of a target sequence, for example, by mediating RNA interference “RNAi” or gene silencing in a sequence-specific manner. As disclosed herein, the dsRNA is capable of reducing or eliminating the level or expression of a target polynucleotide or the polypeptide encoded thereby in a pest.

The dsRNA can reduce or eliminate the expression level of the target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). See, for example, Verdel et al. (2004) Science 303:672-676; Pal-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237. Methods to assay for functional dsRNA that are capable of reducing or eliminating the level of a sequence of interest are disclosed elsewhere herein. Accordingly, as used herein, the term “dsRNA” is meant to encompass other terms used to describe nucleic acid molecules that are capable of mediating RNA interference or gene silencing, including, for example, short-interfering RNA (siRNA), double-stranded RNA (dsRNA), micro-RNA (miRNA), hairpin RNA, short hairpin RNA (shRNA), post-transcriptional gene silencing RNA (ptgsRNA), and others.

In specific embodiments, at least one strand of the duplex or double-stranded region of the dsRNA shares sufficient sequence identity or sequence complementarity to the target polynucleotide to allow for the dsRNA to reduce the level of expression of the target sequence. As used herein, the strand that is complementary to the target polynucleotide is the “antisense strand” and the strand homologous to the target polynucleotide is the “sense strand.”

In another embodiment, the dsRNA comprises a hairpin RNA. A hairpin RNA comprises an RNA molecule that is capable of folding back onto itself to form a double stranded structure. Multiple structures can be employed as hairpin elements. In specific embodiments, the dsRNA suppression element comprises a hairpin element which comprises in the following order, a first segment, a second segment, and a third segment, where the first and the third segment share sufficient complementarity to allow the transcribed RNA to form a double-stranded stem-loop structure.

The “second segment” of the hairpin comprises a “loop” or a “loop region.” These terms are used synonymously herein and are to be construed broadly to comprise any nucleotide sequence that confers enough flexibility to allow self-pairing to occur between complementary regions of a polynucleotide (i.e., segments 1 and 3 which form the stem of the hairpin). For example, in some embodiments, the loop region may be substantially single stranded and act as a spacer between the self-complementary regions of the hairpin stem-loop.

In some embodiments, the loop region can comprise a random or nonsense nucleotide sequence and thus not share sequence identity to a target polynucleotide. In other embodiments, the loop region comprises a sense or an antisense RNA sequence or fragment thereof that shares identity to a target polynucleotide. See, for example, International Patent Publication No. WO 02/00904, herein incorporated by reference. In specific embodiments, the loop region can be optimized to be as short as possible while still providing enough intramolecular flexibility to allow the formation of the base-paired stem region. Accordingly, the loop sequence is generally less than 1000, 900, 800, 700, 600, 500, 400, 300, 200, 100, 50, 25, 20, 19, 18, 17, 16, 15, 10 nucleotides or less.

The “first” and the “third” segment of the hairpin RNA molecule comprise the base-paired stem of the hairpin structure. The first and the third segments are inverted repeats of one another and share sufficient complementarity to allow the formation of the base-paired stem region. In specific embodiments, the first and the third segments are fully complementary to one another. Alternatively, the first and the third segment may be partially complementary to each other so long as they are capable of hybridizing to one another to form a base-paired stem region. The amount of complementarity between the first and the third segment can be calculated as a percentage of the entire segment. Thus, the first and the third segment of the hairpin RNA generally share at least 50%, 60%, 70%, 80%, 85%, 90%, 91%, 92%, 93%, 94%, 95%, 96%, 97%, 98%, 99%, up to and including 100% complementarity.

The first and the third segment are at least about 1000, 500, 475, 450, 425, 400, 375, 350, 325, 300, 250, 225, 200, 175, 150, 125, 100, 75, 60, 50, 40, 30, 25, 22, 20, 19, 18, 17, 16, 15 or 10 nucleotides in length. In specific embodiments, the length of the first and/or the third segment is about 10-100 nucleotides, about 10 to about 75 nucleotides, about 10 to about 50 nucleotides, about 10 to about 40 nucleotides, about 10 to about 35 nucleotides, about 10 to about 30 nucleotides, about 10 to about 25 nucleotides, about 10 to about 19 nucleotides, about 10 to about 20 nucleotides, about 19 to about 50 nucleotides, about 50 nucleotides to about 100 nucleotides, about 100 nucleotides to about 150 nucleotides, about 100 nucleotides to about 300 nucleotides, about 150 nucleotides to about 200 nucleotides, about 200 nucleotides to about 250 nucleotides, about 250 nucleotides to about 300 nucleotides, about 300 nucleotides to about 350 nucleotides, about 350 nucleotides to about 400 nucleotides, about 400 nucleotide to about 500 nucleotides, about 600 nt, about 700 nt, about 800 nt, about 900 nt, about 1000 nt, about 1100 nt, about 1200 nt, 1300 nt, 1400 nt, 1500 nt, 1600 nt, 1700 nt, 1800 nt, 1900 nt, 2000 nt or longer. In other embodiments, the length of the first and/or the third segment comprises at least 10-19 nucleotides, 10-20 nucleotides; 19-35 nucleotides, 20-35 nucleotides; 30-45 nucleotides; 40-50 nucleotides; 50-100 nucleotides; 100-300 nucleotides; about 500-700 nucleotides; about 700-900 nucleotides; about 900-1100 nucleotides; about 1300-1500 nucleotides; about 1500-1700 nucleotides; about 1700-1900 nucleotides; about 1900-2100 nucleotides; about 2100-2300 nucleotides; or about 2300-2500 nucleotides. See, for example, International Publication No. WO 0200904.

Hairpin molecules or double-stranded RNA molecules disclosed herein may have more than one sequence disclosed herein, or active fragments or variants, or complements thereof, found in the same portion of the RNA molecule. For example, in a chimeric hairpin structure, the first segment of a hairpin molecule comprises two polynucleotide sections, each with a different sequence disclosed herein. For example, reading from one terminus of the hairpin, the first segment is composed of sequences from two separate genes (A followed by B). This first segment is followed by the second segment, the loop portion of the hairpin. The loop segment is followed by the third segment, where the complementary strands of the sequences in the first segment are found (B* followed by A*) in forming the stem-loop, hairpin structure, the stem contains SeqA-A* at the distal end of the stem and SeqB-B* proximal to the loop region.

In specific embodiments, the first and the third segment comprise at least 20 nucleotides having at least 85% complementary to the first segment. In still other embodiments, the first and the third segments which form the stem-loop structure of the hairpin comprises 3′ or 5′ overhang regions having unpaired nucleotide residues.

In specific embodiments, the sequences used in the first, the second, and/or the third segments comprise domains that are designed to have sufficient sequence identity to a target polynucleotide of interest and thereby have the ability to decrease the level of expression of the target polynucleotide. The specificity of the inhibitory RNA transcripts is therefore generally conferred by these domains of the silencing element. Thus, in some embodiments of the invention, the first, second and/or third segment of the silencing element comprise a domain having at least 10, at least 15, at least 19, at least 20, at least 21, at least 22, at least 23, at least 24, at least 25, at least 30, at least 40, at least 50, at least 100, at least 200, at least 300, at least 500, at least 1000, or more than 1000 nucleotides that share sufficient sequence identity to the target polynucleotide to allow for a decrease in expression levels of the target polynucleotide when expressed in an appropriate cell. In other embodiments, the domain is between about 15 to 50 nucleotides, about 19-35 nucleotides, about 20-35 nucleotides, about 25-50 nucleotides, about 19 to 75 nucleotides, about 20 to 75 nucleotides, about 40-90 nucleotides about 15-100 nucleotides 10-100 nucleotides, about 10 to about 75 nucleotides, about 10 to about 50 nucleotides, about 10 to about 40 nucleotides, about 10 to about 35 nucleotides, about 10 to about 30 nucleotides, about 10 to about 25 nucleotides, about 10 to about 20 nucleotides, about 10 to about 19 nucleotides, about 50 nucleotides to about 100 nucleotides, about 100 nucleotides to about 150 nucleotides, about 150 nucleotides to about 200 nucleotides, about 200 nucleotides to about 250 nucleotides, about 250 nucleotides to about 300 nucleotides, about 300 nucleotides to about 350 nucleotides, about 350 nucleotides to about 400 nucleotides, about 400 nucleotide to about 500 nucleotides or longer. In other embodiments, the length of the first and/or the third segment comprises at least 10-20 nucleotides, at least 10-19 nucleotides, 20-35 nucleotides, 30-45 nucleotides, 40-50 nucleotides, 50-100 nucleotides, or about 100-300 nucleotides.

In specific embodiments, the domain of the first, the second, and/or the third segment has 100% sequence identity to the target polynucleotide. In other embodiments, the domain of the first, the second and/or the third segment having homology to the target polypeptide have at least 50%, 60%, 70%, 80%, 85%, 90%, 91%, 92%, 93%, 94%, 95%, 96%, 97%, 98%, 99%, or greater sequence identity to a region of the target polynucleotide. The sequence identity of the domains of the first, the second and/or the third segments to the target polynucleotide need only be sufficient to decrease expression of the target polynucleotide of interest. See, for example, Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30:135-140, herein incorporated by reference.

The amount of complementarity shared between the first, second, and/or third segment and the target polynucleotide or the amount of complementarity shared between the first segment and the third segment (i.e., the stem of the hairpin structure) may vary depending on the organism in which gene expression is to be controlled. Some organisms or cell types may require exact pairing or 100% identity, while other organisms or cell types may tolerate some mismatching. In some cells, for example, a single nucleotide mismatch in the targeting sequence abrogates the ability to suppress gene expression. In these cells, the suppression cassettes of the invention can be used to target the suppression of mutant genes, for example, oncogenes whose transcripts comprise point mutations and therefore they can be specifically targeted using the methods and compositions of the invention without altering the expression of the remaining wild-type allele. In other organisms, holistic sequence variability may be tolerated as long as some 22nt region of the sequence is represented in 100% homology between target polynucleotide and the suppression cassette.

Any region of the target polynucleotide can be used to design the domain of the silencing element that shares sufficient sequence identity to allow expression of the hairpin transcript to decrease the level of the target polynucleotide. For instance, the domain can be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof. In specific embodiments, a domain of the silencing element shares sufficient homology to at least about 15, 16, 17, 18, 19, 20, 22, 25 or 30 consecutive nucleotides from about nucleotides 1-50, 25-75, 75-125, 50-100, 125-175, 175-225, 100-150, 150-200, 200-250, 225-275, 275-325, 250-300, 325-375, 375-425, 300-350, 350-400, 425-475, 400-450, 475-525, 450-500, 525-575, 575-625, 550-600, 625-675, 675-725, 600-650, 625-675, 675-725, 650-700, 725-825, 825-875, 750-800, 875-925, 925-975, 850-900, 925-975, 975-1025, 950-1000, 1000-1050, 1025-1075, 1075-1125, 1050-1100, 1125-1175, 1100-1200, 1175-1225, 1225-1275, 1200-1300, 1325-1375, 1375-1425, 1300-1400, 1425-1475, 1475-1525, 1400-1500, 1525-1575, 1575-1625, 1625-1675, 1675-1725, 1725-1775, 1775-1825, 1825-1875, 1875-1925, 1925-1975, 1975-2025, 2025-2075, 2075-2125, 2125-2175, 2175-2225, 1500-1600, 1600-1700, 1700-1800, 1800-1900, 1900-2000 of the target sequence. In some instances to optimize the siRNA sequences employed in the hairpin, the synthetic oligodeoxyribonucleotide/RNAse H method can be used to determine sites on the target mRNA that are in a conformation that is susceptible to RNA silencing. See, for example, Vickers et al. (2003) J. Biol. Chem 278:7108-7118 and Yang et al. (2002) Proc. Natl. Acad. Sci. USA 99:9442-9447, herein incorporated by reference. These studies indicate that there is a significant correlation between the RNase-H-sensitive sites and sites that promote efficient siRNA-directed mRNA degradation.

The hairpin silencing element may also be designed such that the sense sequence or the antisense sequence do not correspond to a target polynucleotide. In this embodiment, the sense and antisense sequence flank a loop sequence that comprises a nucleotide sequence corresponding to all or part of the target polynucleotide. Thus, it is the loop region that determines the specificity of the RNA interference. See, for example, WO 02/00904, herein incorporated by reference.

In addition, transcriptional gene silencing (TGS) may be accomplished through use of a hairpin suppression element where the inverted repeat of the hairpin shares sequence identity with the promoter region of a target polynucleotide to be silenced. See, for example, Aufsatz et al. (2002) PNAS 99 (Suppl. 4):16499-16506 and Mette et al. (2000) EMBO J 19(19):5194-5201.

In other embodiments, the silencing element can comprise a small RNA (sRNA). sRNAs can comprise both micro RNA (miRNA) and short-interfering RNA (siRNA) (Meister and Tuschl (2004) Nature 431:343-349 and Bonetta et al. (2004) Nature Methods 1:79-86). miRNAs are regulatory agents comprising about 19 to about 24 ribonucleotides in length which are highly efficient at inhibiting the expression of target polynucleotides. See, for example Javier et al. (2003) Nature 425: 257-263, herein incorporated by reference. For miRNA interference, the silencing element can be designed to express a dsRNA molecule that forms a hairpin structure or partially base-paired structure containing 19, 20, 21, 22, 23, 24 or 25-nucleotide sequence that is complementary to the target polynucleotide of interest. The miRNA can be synthetically made, or transcribed as a longer RNA which is subsequently cleaved to produce the active miRNA. Specifically, the miRNA can comprise 19 nucleotides of the sequence having homology to a target polynucleotide in sense orientation and 19 nucleotides of a corresponding antisense sequence that is complementary to the sense sequence. The miRNA can be an “artificial miRNA” or “amiRNA” which comprises a miRNA sequence that is synthetically designed to silence a target sequence.

When expressing an miRNA the final (mature) miRNA is present in a duplex in a precursor backbone structure, the two strands being referred to as the miRNA (the strand that will eventually basepair with the target) and miRNA*(star sequence). It has been demonstrated that miRNAs can be transgenically expressed and target genes of interest efficiently silenced (Highly specific gene silencing by artificial microRNAs in Arabidopsis Schwab R, Ossowski S, Riester M, Warthmann N, Weigel D. Plant Cell. 2006 May; 18(5):1121-33. Epub 2006 Mar. 10 & Expression of artificial microRNAs in transgenic Arabidopsis thaliana confers virus resistance. Niu Q W, Lin S S, Reyes J L, Chen K C, Wu H W, Yeh S D, Chua N H. Nat Biotechnol. 2006 November; 24(11):1420-8. Epub 2006 Oct. 22. Erratum in: Nat Biotechnol. 2007 February; 25(2):254.)

The silencing element for miRNA interference comprises a miRNA primary sequence. The miRNA primary sequence comprises a DNA sequence having the miRNA and star sequences separated by a loop as well as additional sequences flanking this region that are important for processing. When expressed as an RNA, the structure of the primary miRNA is such as to allow for the formation of a hairpin RNA structure that can be processed into a mature miRNA. In some embodiments, the miRNA backbone comprises a genomic or cDNA miRNA precursor sequence, wherein said sequence comprises a native primary in which a heterologous (artificial) mature miRNA and star sequence are inserted.

As used herein, a “star sequence” is the sequence within a miRNA precursor backbone that is complementary to the miRNA and forms a duplex with the miRNA to form the stem structure of a hairpin RNA. In some embodiments, the star sequence can comprise less than 100% complementarity to the miRNA sequence. Alternatively, the star sequence can comprise at least 99%, 98%, 97%, 96%, 95%, 90%, 85%, 80% or lower sequence complementarity to the miRNA sequence as long as the star sequence has sufficient complementarity to the miRNA sequence to form a double stranded structure. In still further embodiments, the star sequence comprises a sequence having 1, 2, 3, 4, 5 or more mismatches with the miRNA sequence and still has sufficient complementarity to form a double stranded structure with the miRNA sequence resulting in production of miRNA and suppression of the target sequence.

The miRNA precursor backbones can be from any plant. In some embodiments, the miRNA precursor backbone is from a monocot. In other embodiments, the miRNA precursor backbone is from a dicot. In further embodiments, the backbone is from maize or soybean. MicroRNA precursor backbones have been described previously. For example, US20090155910A1 (WO 2009/079532) discloses the following soybean miRNA precursor backbones: 156c, 159, 166b, 168c, 396b and 398b, and US20090155909A1 (WO 2009/079548) discloses the following maize miRNA precursor backbones: 159c, 164h, 168a, 169r, and 396h. Each of these references is incorporated by reference in their entirety.

Thus, the primary miRNA can be altered to allow for efficient insertion of heterologous miRNA and star sequences within the miRNA precursor backbone. In such instances, the miRNA segment and the star segment of the miRNA precursor backbone are replaced with the heterologous miRNA and the heterologous star sequences, designed to target any sequence of interest, using a PCR technique and cloned into an expression construct. It is recognized that there could be alterations to the position at which the artificial miRNA and star sequences are inserted into the backbone. Detailed methods for inserting the miRNA and star sequence into the miRNA precursor backbone are described in, for example, US Patent Applications 20090155909A1 and US20090155910A1, herein incorporated by reference in their entirety.

When designing a miRNA sequence and star sequence, various design choices can be made. See, for example, Schwab R, et al. (2005) Dev Cell 8: 517-27. In non-limiting embodiments, the miRNA sequences disclosed herein can have a “U” at the 5′-end, a “C” or “G” at the 19th nucleotide position, and an “A” or “U” at the 10th nucleotide position. In other embodiments, the miRNA design is such that the miRNA have a high free delta-G as calculated using the ZipFold algorithm (Markham, N. R. & Zuker, M. (2005) Nucleic Acids Res. 33: W577-W581.) Optionally, a one base pair change can be added within the 5′ portion of the miRNA so that the sequence differs from the target sequence by one nucleotide.

The methods and compositions disclosed herein employ silencing elements that when transcribed “form” a dsRNA molecule. Accordingly, the heterologous polynucleotide being expressed need not form the dsRNA by itself, but can interact with other sequences in the plant cell or in the pest gut after ingestion to allow the formation of the dsRNA. For example, a chimeric polynucleotide that can selectively silence the target polynucleotide can be generated by expressing a chimeric construct comprising the target sequence for a miRNA or siRNA to a sequence corresponding to all or part of the gene or genes to be silenced. In this embodiment, the dsRNA is “formed” when the target for the miRNA or siRNA interacts with the miRNA present in the cell. The resulting dsRNA can then reduce the level of expression of the gene or genes to be silenced. See, for example, US Application Publication 2007-0130653, entitled “Methods and Compositions for Gene Silencing”, herein incorporated by reference. The construct can be designed to have a target for an endogenous miRNA or alternatively, a target for a heterologous and/or synthetic miRNA can be employed in the construct. If a heterologous and/or synthetic miRNA is employed, it can be introduced into the cell on the same nucleotide construct as the chimeric polynucleotide or on a separate construct. As discussed elsewhere herein, any method can be used to introduce the construct comprising the heterologous miRNA.

### IV. Variants and Fragments

By “fragment” is intended a portion of the polynucleotide or a portion of the amino acid sequence and hence protein encoded thereby. Fragments of a polynucleotide may encode protein fragments that retain the biological activity of the native protein. Alternatively, fragments of a polynucleotide that are useful as a silencing element do not need to encode fragment proteins that retain biological activity. Thus, fragments of a nucleotide sequence may range from at least about 10, about 15, about 16, about 17, about 18, about 19, nucleotides, about 20 nucleotides, about 22 nucleotides, about 50 nucleotides, about 75 nucleotides, about 100 nucleotides, 200 nucleotides, 300 nucleotides, 400 nucleotides, 500 nucleotides, 600 nucleotides, 700 nucleotides and up to the full-length polynucleotide employed in the invention. Alternatively, fragments of a nucleotide sequence may range from 1-50, 25-75, 75-125, 50-100, 125-175, 175-225, 100-150, 100-300, 150-200, 200-250, 225-275, 275-325, 250-300, 325-375, 375-425, 300-350, 350-400, 425-475, 400-450, 475-525, 450-500, 525-575, 575-625, 550-600, 625-675, 675-725, 600-650, 625-675, 675-725, 650-700, 725-825, 825-875, 750-800, 875-925, 925-975, 850-900, 925-975, 975-1025, 950-1000, 1000-1050, 1025-1075, 1075-1125, 1050-1100, 1125-1175, 1100-1200, 1175-1225, 1225-1275, 1200-1300, 1325-1375, 1375-1425, 1300-1400, 1425-1475, 1475-1525, 1400-1500, 1525-1575, 1575-1625, 1625-1675, 1675-1725, 1725-1775, 1775-1825, 1825-1875, 1875-1925, 1925-1975, 1975-2025, 2025-2075, 2075-2125, 2125-2175, 2175-2225, 1500-1600, 1600-1700, 1700-1800, 1800-1900, 1900-2000 of any one of SEQ ID NOS.: 1-86 and variants and fragments thereof, and complements thereof. Methods to assay for the activity of a desired silencing element are described elsewhere herein.

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. A variant of a polynucleotide that is useful as a silencing element will retain the ability to reduce expression of the target polynucleotide and, in some embodiments, thereby control a pest of interest. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the polypeptides employed in the invention. Variant polynucleotides also include synthetically derived polynucleotide, such as those generated, for example, by using site-directed mutagenesis, but continue to retain the desired activity. Generally, variants of a particular polynucleotide of the invention (i.e., a silencing element) will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides employed in the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A method is further provided for identifying a silencing element from the target polynucleotides set forth in SEQ ID NOS.: 1-86, variants or fragments thereof, or complements thereof. Such methods comprise obtaining a candidate fragment of any one of SEQ ID NOS.: 1-86,variants or fragments thereof, or complements thereof, which is of sufficient length to act as a silencing element and thereby reduce the expression of the target polynucleotide and/or control a desired pest; expressing said candidate polynucleotide fragment in an appropriate expression cassette to produce a candidate silencing element and determining is said candidate polynucleotide fragment has the activity of a silencing element and thereby reduce the expression of the target polynucleotide and/or controls a desired pest. Methods of identifying such candidate fragments based on the desired pathway for suppression are known. For example, various bioinformatics programs can be employed to identify the region of the target polynucleotides that could be exploited to generate a silencing element. See, for example, Elbahir et al. (2001) Genes and Development 15:188-200, Schwartz et al. (2003) Cell 115:199-208, Khvorova et al. (2003) Cell 115:209-216. See also, siRNA at Whitehead (jura.wi.mit.edu/bioc/siRNAext/) which calculates the binding energies for both sense and antisense siRNAs. See, also genscript.com/ssl-bin/app/mai?op=known; Block-iT™ RNAi designer from Invitrogen and GenScript siRNA Construct Builder. In various aspects, it is to be understand that the term “ . . . SEQ ID NOs: 1-86, or variants or fragments thereof, or complements thereof . . . ” is intended to mean that the disclosed sequences comprise SEQ ID NOs: 1-86, and/or fragments of SEQ ID NOs: 1-86, and/or variants of SEQ ID NOs: 1-86, and/or the complements of SEQ ID NOs: 1-86, the variants of SEQ ID NOs: 1-86, and/or the fragments of SEQ ID NOs: 1-86, individually (or) or inclusive of some or all listed sequences.

### V. DNA Constructs

The use of the term “polynucleotide” is not intended to limit the disclosed polynucleotides to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The polynucleotide encoding the silencing element or in specific embodiments employed in the methods and compositions of the invention can be provided in expression cassettes for expression in a plant or organism of interest. It is recognized that multiple silencing elements including multiple identical silencing elements, multiple silencing elements targeting different regions of the target sequence, or multiple silencing elements from different target sequences can be used. In this embodiment, it is recognized that each silencing element can be contained in a single or separate cassette, DNA construct, or vector. As discussed, any means of providing the silencing element is contemplated. A plant or plant cell can be transformed with a single cassette comprising DNA encoding one or more silencing elements or separate cassettes comprising each silencing element can be used to transform a plant or plant cell or host cell. Likewise, a plant transformed with one component can be subsequently transformed with the second component. One or more silencing elements can also be brought together by sexual crossing. That is, a first plant comprising one component is crossed with a second plant comprising the second component. Progeny plants from the cross will comprise both components.

The expression cassette can include 5′ and 3′ regulatory sequences operably linked to the polynucleotide of the invention. “Operably linked” is intended to mean a functional linkage between two or more elements. For example, an operable linkage between a polynucleotide of the invention and a regulatory sequence (i.e., a promoter) is a functional link that allows for expression of the polynucleotide of the invention. Operably linked elements may be contiguous or non-contiguous. When used to refer to the joining of two protein coding regions, by operably linked is intended that the coding regions are in the same reading frame. The cassette may additionally contain at least one additional polynucleotide to be cotransformed into the organism. Alternatively, the additional polypeptide(s) can be provided on multiple expression cassettes. Expression cassettes can be provided with a plurality of restriction sites and/or recombination sites for insertion of the polynucleotide to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a polynucleotide comprising the silencing element employed in the methods and compositions of the invention, and a transcriptional and translational termination region (i.e., termination region) functional in plants. In other embodiment, the double stranded RNA is expressed from a suppression cassette. Such a cassette can comprise two convergent promoters that drive transcription of an operably linked silencing element. “Convergent promoters” refers to promoters that are oriented on either terminus of the operably linked silencing element such that each promoter drives transcription of the silencing element in opposite directions, yielding two transcripts. In such embodiments, the convergent promoters allow for the transcription of the sense and anti-sense strand and thus allow for the formation of a dsRNA. Such a cassette may also comprise two divergent promoters that drive transcription of one or more operably linked silencing elements. “Divergent promoters” refers to promoters that are oriented in opposite directions of each other, driving transcription of the one or more silencing elements in opposite directions. In such embodiments, the divergent promoters allow for the transcription of the sense and antisense strands and allow for the formation of a dsRNA. In such embodiments, the divergent promoters also allow for the transcription of at least two separate hairpin RNAs. In another embodiment, one cassette comprising two or more silencing elements under the control of two separate promoters in the same orientation is present in a construct. In another embodiment, two or more individual cassettes, each comprising at least one silencing element under the control of a promoter, are present in a construct in the same orientation.

The regulatory regions (i.e., promoters, transcriptional regulatory regions, and translational termination regions) and/or the polynucleotides employed in the invention may be native/analogous to the host cell or to each other. Alternatively, the regulatory regions and/or the polynucleotide employed in the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

The termination region may be native with the transcriptional initiation region, may be native with the operably linked polynucleotide encoding the silencing element, may be native with the plant host, or may be derived from another source (i.e., foreign or heterologous) to the promoter, the polynucleotide comprising silencing element, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The polynucleotide encoding the silencing element can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

An inducible promoter, for instance, a pathogen-inducible promoter could also be employed. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10): 1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179.

In an embodiment, the plant-expressed promoter is a vascular-specific promoter such as a phloem-specific promoter. A “vascular-specific” promoter, as used herein, is a promoter which is at least expressed in vascular cells, or a promoter which is preferentially expressed in vascular cells. Expression of a vascular-specific promoter need not be exclusively in vascular cells, expression in other cell types or tissues is possible. A “phloem-specific promoter” as used herein, is a plant-expressible promoter which is at least expressed in phloem cells, or a promoter which is preferentially expressed in phloem cells.

Expression of a phloem-specific promoter need not be exclusively in phloem cells, expression in other cell types or tissues, e.g., xylem tissue, is possible. In one embodiment of this invention, a phloem-specific promoter is a plant-expressible promoter at least expressed in phloem cells, wherein the expression in non-phloem cells is more limited (or absent) compared to the expression in phloem cells. Examples of suitable vascular-specific or phloem-specific promoters in accordance with this invention include but are not limited to the promoters selected from the group consisting of: the SCSV3, SCSV4, SCSV5, and SCSV7 promoters (Schunmann et al. (2003) Plant Functional Biology 30:453-60; the rolC gene promoter of Agrobacterium rhizogenes (Kiyokawa et al. (1994) Plant Physiology 104:801-02; Pandolfini et al. (2003) BioMedCentral (BMC)Biotechnology 3:7, (www.biomedcentral.com/1472-6750/3/7); Graham et al. (1997) Plant Mol. Biol. 33:729-35; Guivarc'h et al. (1996); Almon et al. (1997) Plant Physiol. 115:1599-607; the rolA gene promoter of Agrobacterium rhizogenes (Dehio et al. (1993) Plant Mol. Biol. 23:1199-210); the promoter of the Agrobacterium tumefaciens T-DNA gene 5 (Korber et al. (1991) EMBO J. 10:3983-91); the rice sucrose synthase RSs1 gene promoter (Shi et al. (1994) J. Exp. Bot. 45:623-31); the CoYMV or Commelina yellow mottle badnavirus promoter (Medberry et al. (1992) Plant Cell 4:185-92; Zhou et al. (1998) Chin. J. Biotechnol. 14:9-16); the CFDV or coconut foliar decay virus promoter (Rohde et al. (1994) Plant Mol. Biol. 27:623-28; Hehn and Rhode (1998) J. Gen. Virol. 79:1495-99); the RTBV or rice tungro bacilliform virus promoter (Yin and Beachy (1995) Plant J. 7:969-80; Yin et al. (1997) Plant J. 12:1179-80); the pea glutamin synthase GS3A gene (Edwards et al. (1990) Proc. Natl. Acad. Sci. USA 87:3459-63; Brears et al. (1991) Plant J. 1:235-44); the inv CD111 and inv CD141 promoters of the potato invertase genes (Hedley et al. (2000) J. Exp. Botany 51:817-21); the promoter isolated from Arabidopsis shown to have phloem-specific expression in tobacco by Kertbundit et al. (1991) Proc. Natl. Acad. Sci. USA 88:5212-16); the VAHOX1 promoter region (Tornero et al. (1996) Plant J. 9:639-48); the pea cell wall invertase gene promoter (Zhang et al. (1996) Plant Physiol. 112:1111-17); the promoter of the endogenous cotton protein related to chitinase of US published patent application 20030106097, an acid invertase gene promoter from carrot (Ramloch-Lorenz et al. (1993) The Plant J. 4:545-54); the promoter of the sulfate transporter geneSultr1; 3 (Yoshimoto et al. (2003) Plant Physiol. 131:1511-17); a promoter of a sucrose synthase gene (Nolte and Koch (1993) Plant Physiol. 101:899-905); and the promoter of a tobacco sucrose transporter gene (Kuhn et al. (1997) Science 275-1298-1300).

Possible promoters also include the Black Cherry promoter for Prunasin Hydrolase (PH DL1.4 PRO) (U.S. Pat. No. 6,797,859), Thioredoxin H promoter from cucumber and rice (Fukuda A et al. (2005). Plant Cell Physiol. 46(11):1779-86), Rice (RSs1) (Shi, T. Wang et al. (1994). J. Exp. Bot. 45(274): 623-631) and maize sucrose synthese-1 promoters (Yang, N-S. et al. (1990) PNAS 87:4144-4148), PP2 promoter from pumpkin Guo, H. et al. (2004) Transgenic Research 13:559-566), At SUC2 promoter (Truernit, E. et al. (1995) Planta 196(3):564-70. At SAM-1 (S-adenosylmethionine synthetase) (Mijnsbrugge K V. et al. (1996) Planr. Cell. Physiol. 37(8): 1108-1115), and the Rice tungro bacilliform virus (RTBV) promoter (Bhattacharyya-Pakrasi et al. (1993) Plant J. 4(1):71-79).

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Bairnm et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not meant to be limiting. Any selectable marker gene can be used with the disclosed polynucleotides, constructs, vectors, methods, and compositions.

### VI. Compositions Comprising Silencing Elements

One or more of the polynucleotides comprising the silencing element can be provided as an external composition such as a spray or powder to the plant, plant part, seed, a pest, or an area of cultivation. In another example, a plant is transformed with a DNA construct or expression cassette for expression of at least one silencing element. In either composition, the silencing element, when ingested by an insect, can reduce the level of a target pest sequence and thereby control the pest (i.e., a Coleopteran plant pest including a Diabrotica plant pest, such as, D. virgifera virgifera, D. barberi, D. virgifera zeae, D. speciosa, or D. undecimpunctata howardi). It is recognized that the composition can comprise a cell (such as plant cell or a bacterial cell), in which a polynucleotide encoding the silencing element is stably incorporated into the genome and operably linked to promoters active in the cell. Compositions comprising a mixture of cells, some cells expressing at least one silencing element are also encompassed. In other embodiments, compositions comprising the silencing elements are not contained in a cell. In such embodiments, the composition can be applied to an area inhabited by a pest. In one embodiment, the composition is applied externally to a plant (i.e., by spraying a field or area of cultivation) to protect the plant from the pest. Methods of applying nucleotides in such a manner are known to those of skill in the art.

The composition of the invention can further be formulated as bait. In this embodiment, the compositions comprise a food substance or an attractant which enhances the attractiveness of the composition to the pest.

The composition comprising the silencing element can be formulated in an agriculturally suitable and/or environmentally acceptable carrier. Such carriers can be any material that the animal, plant or environment to be treated can tolerate. Furthermore, the carrier must be such that the composition remains effective at controlling a pest. Examples of such carriers include water, saline, Ringer's solution, dextrose or other sugar solutions, Hank's solution, and other aqueous physiologically balanced salt solutions, phosphate buffer, bicarbonate buffer and Tris buffer. In addition, the composition may include compounds that increase the half-life of a composition. Various insecticidal formulations can also be found in, for example, US Publications 2008/0275115, 2008/0242174, 2008/0027143, 2005/0042245, and 2004/0127520, each of which is herein incorporated by reference.

It is recognized that the polynucleotides comprising sequences encoding the silencing element can be used to transform organisms to provide for host organism production of these components, and subsequent application of the host organism to the environment of the target pest(s). Such host organisms include baculoviruses, bacteria, and the like. In this manner, the combination of polynucleotides encoding the silencing element may be introduced via a suitable vector into a microbial host, and said host applied to the environment, or to plants or animals.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be stably incorporated into the genome of the cell (e.g., chromosome, plasmid, plastid, or mitochondrial DNA), converted into an autonomous replicon, or transiently expressed (e.g., transfected mRNA).

Microbial hosts that are known to occupy the “phytosphere” (phylloplane, phyllosphere, rhizosphere, and/or rhizoplana) of one or more crops of interest may be selected. These microorganisms are selected so as to be capable of successfully competing in the particular environment with the wild-type microorganisms, provide for stable maintenance and expression of the sequences encoding the silencing element, and desirably, provide for improved protection of the components from environmental degradation and inactivation.

Such microorganisms include bacteria, algae, and fungi. Of particular interest are microorganisms such as bacteria, e.g., Pseudomonas, Erwinia, Serratia, Klebsiella, Xanthomonas, Streptomyces, Rhizobium, Rhodopseudomonas, Methylius, Agrobacterium, Acetobacter, Lactobacillus, Arthrobacter, Azotobacter, Leuconostoc, and Alcaligenes, fungi, particularly yeast, e.g., Saccharomyces, Cryptococcus, Kluyveromyces, Sporobolomyces, Rhodotorula, and Aureobasidium. Of particular interest are such phytosphere bacterial species as Pseudomonas syringae, Pseudomonas fluorescens, Serratia marcescens, Acetobacter xylinum, Agrobacteria, Rhodopseudomonas spheroides, Xanthomonas campestris, Rhizobium melioti, Alcaligenes entrophus, Clavibacter xyli and Azotobacter vinlandir, and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces rosues, S. odorus, Kluyveromyces veronae, and Aureobasidium pollulans. Of particular interest are the pigmented microorganisms.

A number of ways are available for introducing the polynucleotide comprising the silencing element into the microbial host under conditions that allow for stable maintenance and expression of such nucleotide encoding sequences. For example, expression cassettes can be constructed which include the nucleotide constructs of interest operably linked with the transcriptional and translational regulatory signals for expression of the nucleotide constructs, and a nucleotide sequence homologous with a sequence in the host organism, whereby integration will occur, and/or a replication system that is functional in the host, whereby integration or stable maintenance will occur.

Transcriptional and translational regulatory signals include, but are not limited to, promoters, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (2000); Molecular Cloning: A Laboratory Manual (3^{rd} ed.; Cold Spring Harbor Laboratory Press, Plainview, N.Y.); Davis et al. (1980) Advanced Bacterial Genetics (Cold Spring Harbor Laboratory, Cold Spring Harbor, N.Y.); and the references cited therein.

Suitable host cells include the prokaryotes and the lower eukaryotes, such as fungi.

Illustrative prokaryotes, both Gram-negative and Gram-positive, include Enterobacteriaceae, such as Escherichia, Erwinia, Shigella, Salmonella, and Proteus; Bacillaceae; Rhizobiceae, such as Rhizobium; Spirillaceae, such as photobacterium, Zymomonas, Serratia, Aeromonas, Vibrio, Desulfovibrio, Spirillum; Lactobacillaceae; Pseudomonadaceae, such as Pseudomonas and Acetobacter; Azotobacteraceae and Nitrobacteraceae. Among eukaryotes are fungi, such as Phycomycetes and Ascomycetes, which includes yeast, such as Saccharomyces and Schizosaccharomyces; and Basidiomycetes yeast, such as Rhodotorula, Aureobasidium, Sporobolomyces, and the like.

Characteristics of particular interest in selecting a host cell for purposes of the invention include ease of introducing the coding sequence into the host, availability of expression systems, efficiency of expression, stability in the host, and the presence of auxiliary genetic capabilities. Characteristics of interest for use as a pesticide microcapsule include protective qualities, such as thick cell walls, pigmentation, and intracellular packaging or formation of inclusion bodies; leaf affinity; lack of mammalian toxicity; attractiveness to pests for ingestion; and the like. Other considerations include ease of formulation and handling, economics, storage stability, and the like.

Host organisms of particular interest include yeast, such as Rhodotorula spp., Aureobasidium spp., Saccharomyces spp., and Sporobolomyces spp., phylloplane organisms such as Pseudomonas spp., Erwinia spp., and Flavobacterium spp., and other such organisms, including Pseudomonas aeruginosa, Pseudomonas fluorescens, Saccharomyces cerevisiae, Bacillus thuringiensis, Escherichia coli, Bacillus subtilis, and the like.

The sequences encoding the silencing elements encompassed by the invention can be introduced into microorganisms that multiply on plants (epiphytes) to deliver these components to potential target pests. Epiphytes, for example, can be gram-positive or gram-negative bacteria.

The silencing element can be fermented in a bacterial host and the resulting bacteria processed and used as a microbial spray in the same manner that Bacillus thuringiensis strains have been used as insecticidal sprays. Any suitable microorganism can be used for this purpose. By way of example, Pseudomonas has been used to express Bacillus thuringiensis endotoxins as encapsulated proteins and the resulting cells processed and sprayed as an insecticide Gaertner et al. (1993), in Advanced Engineered Pesticides, ed. L. Kim (Marcel Decker, Inc.).

Alternatively, the components of the invention are produced by introducing heterologous genes into a cellular host. Expression of the heterologous sequences results, directly or indirectly, in the intracellular production of the silencing element. These compositions may then be formulated in accordance with conventional techniques for application to the environment hosting a target pest, e.g., soil, water, and foliage of plants. See, for example, EPA 0192319, and the references cited therein.

As disclosed herein, a transformed microorganism can be formulated with an acceptable carrier into separate or combined compositions that are, for example, a suspension, a solution, an emulsion, a dusting powder, a dispersible granule, a wettable powder, and an emulsifiable concentrate, an aerosol, an impregnated granule, an adjuvant, a coatable paste, and also encapsulations in, for example, polymer substances.

Such compositions disclosed above may be obtained by the addition of a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors, or other preparations that influence plant growth. One or more agrochemicals including, but not limited to, herbicides, insecticides, fungicides, bactericides, nematicides, molluscicides, acaracides, plant growth regulators, harvest aids, and fertilizers, can be combined with carriers, surfactants or adjuvants customarily employed in the art of formulation or other components to facilitate product handling and application for particular target pests. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients disclosed herein (i.e., at least one silencing element) are normally applied in the form of compositions and can be applied to the crop area, plant, or seed to be treated. For example, the compositions may be applied to grain in preparation for or during storage in a grain bin or silo, etc. The compositions may be applied simultaneously or in succession with other compounds. Methods of applying an active ingredient or a composition that contains at least one silencing element include, but are not limited to, foliar application, seed coating, and soil application. The number of applications and the rate of application depend on the intensity of infestation by the corresponding pest.

Suitable surface-active agents include, but are not limited to, anionic compounds such as a carboxylate of, for example, a metal; carboxylate of a long chain fatty acid; an N-acylsarcosinate; mono- or di-esters of phosphoric acid with fatty alcohol ethoxylates or salts of such esters; fatty alcohol sulfates such as sodium dodecyl sulfate, sodium octadecyl sulfate, or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphthalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate or dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g., polyoxyethylene sorbitan fatty acid esters, block copolymers of ethylene oxide and propylene oxide, acetylenic glycols such as 2,4,7,9-tetraethyl-5-decyn-4,7-diol, or ethoxylated acetylenic glycols. Examples of a cationic surface-active agent include, for instance, an aliphatic mono-, di-, or polyamine such as an acetate, naphthenate or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials include, but are not limited to, inorganic minerals such as kaolin, phyllosilicates, carbonates, sulfates, phosphates, or botanical materials such as cork, powdered corncobs, peanut hulls, rice hulls, and walnut shells.

The compositions comprising the silencing element can be in a suitable form for direct application or as a concentrate of primary composition that requires dilution with a suitable quantity of water or other dilutant before application.

The compositions (including the transformed microorganisms) can be applied to the environment of a plant insect pest (such as a Coleoptera plant pest or a Diabrotica plant pest) by, for example, spraying, atomizing, dusting, scattering, coating or pouring, introducing into or on the soil, introducing into irrigation water, by seed treatment or general application or dusting at the time when the pest has begun to appear or before the appearance of pests as a protective measure. For example, the composition(s) and/or transformed microorganism(s) may be mixed with grain to protect the grain during storage. It is generally important to obtain good control of pests in the early stages of plant growth, as this is the time when the plant can be most severely damaged. The compositions can conveniently contain another insecticide if this is thought necessary. In an embodiment of the invention, the composition(s) is applied directly to the soil, at a time of planting, in granular form of a composition of a carrier and dead cells of a Bacillus strain or transformed microorganism of the invention. Another embodiment is a granular form of a composition comprising an agrochemical such as, for example, an herbicide, an insecticide, a fertilizer, in an inert carrier, and dead cells of a Bacillus strain or transformed microorganism of the invention.

VII. Plants, Plant Parts, and Methods of Introducing Sequences into Plants

In one embodiment, the methods of the invention involve introducing a polynucleotide into a plant. “Introducing” is intended to mean presenting to the plant the polynucleotide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. Nos. 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the silencing element sequences of the invention can be provided to a plant using a variety of transient transformation methods. Such transient transformation methods include, but are not limited to, the introduction of the protein or variants and fragments thereof directly into the plant or the introduction of the transcript into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, polynucleotides can be transiently transformed into the plant using techniques known in the art. Such techniques include viral vector systems and the precipitation of the polynucleotide in a manner that precludes subsequent release of the DNA. Thus, the transcription from the particle-bound DNA can occur, but the frequency with which it is released to become integrated into the genome is greatly reduced. Such methods include the use of particles coated with polyethylimine (PEI; Sigma #P3143).

In other embodiments, the polynucleotide of the invention may be introduced into plants by contacting plants with a virus or viral nucleic acids. Generally, such methods involve incorporating a nucleotide construct of the invention within a viral DNA or RNA molecule. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, provided herein are transformed seed (also referred to as “transgenic seed”) having a polynucleotide of the invention, for example, an expression cassette of the invention, stably incorporated into their genome.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The compositions, methods, constructs, and polynucleotides may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the disclosed methods and compositions include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific aspects, the disclosed plants can be crop plants (for example, corn, alfalfa, sunflower, Brassica, soybean, cotton, safflower, peanut, sorghum, wheat, millet, tobacco, etc.). In other embodiments, corn and soybean plants and sugarcane plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

### VIII. Stacking of Traits in Transgenic Plant

Transgenic plants may comprise a stack of one or more target polynucleotides as set forth in SEQ ID NOS.: 1-86 or variants or fragments thereof, or complements thereof, as disclosed herein with one or more additional polynucleotides resulting in the production or suppression of multiple polypeptide sequences. Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising an expression construct comprising various target polynucleotides as set forth in SEQ ID NOS.: 1-86 or variants or fragments thereof, or complements thereof, as disclosed herein with a subsequent gene and co-transformation of genes into a single plant cell. As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. Co-transformation of polynucleotides can be carried out using single transformation vectors comprising multiple polynucleotides or polynucleotides carried separately on multiple vectors. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. It is further recognized that polynucleotide sequences can be stacked at a desired genomic location using a site-specific recombination system. See, for example, WO 1999/25821, WO 1999/25854, WO 1999/25840, WO 1999/25855 and WO 1999/25853, all of which are herein incorporated by reference.

In some embodiments the various target polynucleotides as set forth in SEQ ID NOS.: 1-86 or variants or fragments thereof, or complements thereof, as disclosed herein, alone or stacked with one or more additional insect resistance traits can be stacked with one or more additional input traits (e.g., herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, and the like). Thus, the polynucleotide embodiments can be used to provide a complete agronomic package of improved crop quality with the ability to flexibly and cost effectively control any number of agronomic pests.

Transgenes useful for stacking include, but are not limited to, to those as described herein below.

i. Transgenes that Confer Resistance to Insects or Disease

(A) Plant disease resistance genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. See, for example, Jones, et al., (1994) Science 266:789 (cloning of the tomato Cf-9 gene for resistance to Cladosporium fulvum); Martin, et al., (1993) Science 262:1432 (tomato Pto gene for resistance to Pseudomonas syringae pv. tomato encodes a protein kinase); Mindrinos, et al., (1994) Cell 78:1089 (Arabidopsis RSP2 gene for resistance to Pseudomonas syringae), McDowell and Woffenden, (2003) Trends Biotechnol. 21(4):178-83 and Toyoda, et al., (2002) Transgenic Res. 11(6):567-82. A plant resistant to a disease is one that is more resistant to a pathogen as compared to the wild type plant.

(B) Genes encoding a Bacillus thuringiensis protein, a derivative thereof or a synthetic polypeptide modeled thereon. See, for example, Geiser, et al., (1986) Gene 48:109, who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC Accession Numbers 40098, 67136, 31995 and 31998. Other non-limiting examples of Bacillus thuringiensis transgenes being genetically engineered are given in the following patents and patent applications and hereby are incorporated by reference for this purpose: U.S. Pat. Nos. 5,188,960; 5,689,052; 5,880,275; 5,986,177; 6,023,013, 6,060,594, 6,063,597, 6,077,824, 6,620,988, 6,642,030, 6,713,259, 6,893,826, 7,105,332; 7,179,965, 7,208,474; 7,227,056, 7,288,643, 7,323,556, 7,329,736, 7,449,552, 7,468,278, 7,510,878, 7,521,235, 7,544,862, 7,605,304, 7,696,412, 7,629,504, 7,705,216, 7,772,465, 7,790,846, 7,858,849 and WO 1991/14778; WO 1999/31248; WO 2001/12731; WO 1999/24581 and WO 1997/40162.

Genes encoding pesticidal proteins may also be stacked including but are not limited to: insecticidal proteins from Pseudomonas sp. such as PSEEN3174 (Monalysin, (2011) PLoS Pathogens, 7:1-13), from Pseudomonas protegens strain CHA0 and Pf-5 (previously fluorescens) (Pechy-Tarr, (2008) Environmental Microbiology 10:2368-2386: Gen Bank Accession No. EU400157); from Pseudomonas Taiwanensis (Liu, et al., (2010) J. Agric. Food Chem. 58:12343-12349) and from Pseudomonas pseudoalcligenes (Zhang, et al., (2009) Annals of Microbiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxinology Journal 3:101-118 and Morgan, et al., (2001) Applied and Envir. Micro. 67:2062-2069), U.S. Pat. No. 6,048,838, and U.S. Pat. No. 6,379,946; and .delta.-endotoxins including, but not limited to, the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry 51 and Cry55 classes of delta-endotoxin genes and the B. thuringiensis cytolytic Cyt1 and Cyt2 genes. Members of these classes of B. thuringiensis insecticidal proteins include, but are not limited to Cry1Aa1 (Accession #Accession #M11250), Cry1Aa2 (Accession #M10917), Cry1Aa3 (Accession #D00348), Cry1Aa4 (Accession #X13535), Cry1Aa5 (Accession #D17518), Cry1Aa6 (Accession #U43605), Cry1Aa7 (Accession #AF081790), Cry1Aa8 (Accession #I26149), Cry1Aa9 (Accession #AB026261), Cry1Aa10 (Accession #AF154676), Cry1Aa11 (Accession #Y09663), Cry1Aa12 (Accession #AF384211), Cry1Aa13 (Accession #AF510713), Cry1Aa14 (Accession #AY197341), Cry1Aa15 (Accession #DQ062690), Cry1Ab1 (Accession #M13898), Cry1Ab2 (Accession #M12661), Cry1Ab3 (Accession #M15271), Cry1Ab4 (Accession #D00117), Cry1Ab5 (Accession #X04698), Cry1Ab6 (Accession #M37263), Cry1Ab7 (Accession #X13233), Cry1Ab8 (Accession #M16463), Cry1Ab9 (Accession #X54939), Cry1Ab10 (Accession #A29125), Cry1Ab11 (Accession #I12419), Cry1Ab12 (Accession #AF059670), Cry1Ab13 (Accession #AF254640), Cry1Ab14 (Accession #U94191), Cry1Ab15 (Accession #AF358861), Cry1Ab16 (Accession #AF375608), Cry1Ab17 (Accession #AAT46415), Cry1Ab18 (Accession #AAQ88259), Cry1Ab19 (Accession #AY847289), Cry1Ab20 (Accession #DQ241675), Cry1Ab21 (Accession #EF683163), Cry1Ab22 (Accession #ABW87320), Cry1Ab-like (Accession #AF327924), Cry1Ab-like (Accession #AF327925), Cry1Ab-like (Accession #AF327926), Cry1Ab-like (Accession #DQ781309), Cry1Ac1 (Accession #M11068), Cry1Ac2 (Accession #M35524), Cry1Ac3 (Accession #X54159), Cry1Ac4 (Accession #M73249), Cry1Ac5 (Accession #M73248), Cry1Ac6 (Accession #U43606), Cry1Ac7 (Accession #U87793), Cry1Ac8 (Accession #U87397), Cry1Ac9 (Accession #U89872), Cry1Ac10 (Accession #AJ002514), Cry1Ac11 (Accession #AJ130970), Cry1Ac12 (Accession #I12418), Cry1Ac13 (Accession #AF148644), Cry1Ac14 (Accession #AF492767), Cry1Ac15 (Accession #AY122057), Cry1Ac16 (Accession #AY730621), Cry1Ac17 (Accession #AY925090), Cry1Ac18 (Accession #DQ023296), Cry1Ac19 (Accession #DQ195217), Cry1Ac20 (Accession #DQ285666), Cry1Ac21 (Accession #DQ062689), Cry1Ac22 (Accession #EU282379), Cry1Ac23 (Accession #AM949588), Cry1Ac24 (Accession #ABL01535), Cry1Ad1 (Accession #M73250), Cry1Ad2 (Accession #A27531), Cry1Ae1 (Accession #M65252), Cry1Af1 (Accession #U82003), Cry1Ag1 (Accession #AF081248), Cry1Ah1 (Accession #AF281866), Cry1Ah2 (Accession #DQ269474), Cry1Ai1 (Accession #AY174873), Cry1A-like (Accession #AF327927), Cry1Ba1 (Accession #X06711), Cry1Ba2 (Accession #X95704), Cry1Ba3 (Accession #AF368257), Cry1Ba4 (Accession #AF363025), Cry1Ba5 (Accession #AB020894), Cry1Ba6 (Accession #ABL60921), Cry1Bb1 (Accession #L32020), Cry1Bc1 (Accession #Z46442), Cry1Bd1 (Accession #U70726), Cry1Bd2 (Accession #AY138457), Cry1Be1 (Accession #AF077326), Cry1Be2 (Accession #AAQ52387), Cry1Bf1 (Accession #AX189649), Cry1Bf2 (Accession #AAQ52380), Cry1Bg1 (Accession #AY176063), Cry1Ca1 (Accession #X07518), Cry1Ca2 (Accession #X13620), Cry1Ca3 (Accession #M73251), Cry1Ca4 (Accession #A27642), Cry1Ca5 (Accession #X96682), Cry1Ca6 [1] (Accession #AF215647), Cry1Ca7 (Accession #AY015492), Cry1Ca8 (Accession #AF362020), Cry1Ca9 (Accession #AY078160), Cry1Ca10 (Accession #AF540014), Cry1Ca11 (Accession #AY955268), Cry1Cb1 (Accession #M97880), Cry1Cb2 (Accession #AY007686), Cry1Cb3 (Accession #EU679502), Cry1Cb-like (Accession #AAX63901), Cry1Da1 (Accession #X54160), Cry1Da2 (Accession #I76415), Cry1Db1 (Accession #Z22511), Cry1 Db2 (Accession #AF358862), Cry1 Dc1 (Accession #EF059913), Cry1Ea1 (Accession #X53985), Cry1Ea2 (Accession #X56144), Cry1Ea3 (Accession #M73252), Cry1Ea4 (Accession #U94323), Cry1Ea5 (Accession #A15535), Cry1Ea6 (Accession #AF202531), Cry1 Ea7 (Accession #AAW72936), Cry1 Ea8 (Accession #ABX11258), Cry1Eb1 (Accession #M73253), Cry1Fa1 (Accession #M63897), Cry1Fa2 (Accession #M73254), Cry1Fb1 (Accession #Z22512), Cry1Fb2 (Accession #AB012288), Cry1Fb3 (Accession #AF062350), Cry1Fb4 (Accession #I73895), Cry1Fb5 (Accession #AF336114), Cry1Fb6 (Accession #EU679500), Cry1Fb7 (Accession #EU679501), Cry1Ga1 (Accession #Z22510), Cry1Ga2 (Accession #Y09326), Cry1Gb1 (Accession #U70725), Cry1Gb2 (Accession #AF288683), Cry1Gc (Accession #AAQ52381), Cry1Ha1 (Accession #Z22513), Cry1Hb1 (Accession #U35780), Cry1H-like (Accession #AF182196), Cry1Ia1 (Accession #X62821), Cry1Ia2 (Accession #M98544), Cry1Ia3 (Accession #L36338), Cry1Ia4 (Accession #L49391), Cry1Ia5 (Accession #Y08920), Cry1Ia6 (Accession #AF076953), Cry1Ia7 (Accession #AF278797), Cry1Ia8 (Accession #AF373207), Cry1Ia9 (Accession #AF521013), Cry1Ia10 (Accession #AY262167), Cry1Ia11 (Accession #AJ315121), Cry1Ia12 (Accession #AAV53390), Cry1Ia13 (Accession #ABF83202), Cry1Ia14 (Accession #EU887515), Cry1Ib1 (Accession #U07642), Cry1Ib2 (Accession #ABW88019), Cry1Ib3 (Accession #EU677422), Cry1Ic1 (Accession #AF056933), Cry1Ic2 (Accession #AAE71691), Cry1Id1 (Accession #AF047579), Cry1Ie1 (Accession #AF211190), Cry1If1 (Accession #AAQ52382), Cry1I-like (Accession #I90732), Cry1I-like (Accession #DQ781310), Cry1Ja1 (Accession #L32019), Cry1Jb1 (Accession #U31527), Cry1Jc1 (Accession #I90730), Cry1Jc2 (Accession #AAQ52372), Cry1Jd1 (Accession #AX189651), Cry1Ka1 (Accession #U28801), Cry1La1 (Accession #AAS60191), Cry1-like (Accession #I90729), Cry2Aa1 (Accession #M31738), Cry2Aa2 (Accession #M23723), Cry2Aa3 (Accession #D86064), Cry2Aa4 (Accession #AF047038), Cry2Aa5 (Accession #AJ 132464), Cry2Aa6 (Accession #AJ 132465), Cry2Aa7 (Accession #AJ132463), Cry2Aa8 (Accession #AF252262), Cry2Aa9 (Accession #AF273218), Cry2Aa10 (Accession #AF433645), Cry2Aa11 (Accession #AAQ52384), Cry2Aa12 (Accession #DQ977646), Cry2Aa13 (Accession #ABL01536), Cry2Aa14 (Accession #ACF04939), Cry2Ab1 (Accession #M23724), Cry2Ab2 (Accession #X55416), Cry2Ab3 (Accession #AF164666), Cry2Ab4 (Accession #AF336115), Cry2Ab5 (Accession #AF441855), Cry2Ab6 (Accession #AY297091), Cry2Ab7 (Accession #DQ119823), Cry2Ab8 (Accession #DQ361266), Cry2Ab9 (Accession #DQ341378), Cry2Ab10 (Accession #EF157306), Cry2Ab11 (Accession #AM691748), Cry2Ab12 (Accession #ABM21764), Cry2Ab13 (Accession #EU909454), Cry2Ab14 (Accession #EU909455), Cry2Ac1 (Accession #X57252), Cry2Ac2 (Accession #AY007687), Cry2Ac3 (Accession #AAQ52385), Cry2Ac4 (Accession #DQ361267), Cry2Ac5 (Accession #DQ341379), Cry2Ac6 (Accession #DQ359137), Cry2Ac7 (Accession #AM292031), Cry2Ac8 (Accession #AM421903), Cry2Ac9 (Accession #AM421904), Cry2Ac10 (Accession #BI 877475), Cry2Ac11 (Accession #AM689531), Cry2Ac12 (Accession #AM689532), Cry2Ad1 (Accession #AF200816), Cry2Ad2 (Accession #DQ358053), Cry2Ad3 (Accession #AM268418), Cry2Ad4 (Accession #AM490199), Cry2Ad5 (Accession #AM765844), Cry2Ae1 (Accession #AAQ52362), Cry2Af1 (Accession #EF439818), Cry2Ag (Accession #ACH91610), Cry2Ah (Accession #EU939453), Cry3Aa1 (Accession #M22472), Cry3Aa2 (Accession #J02978), Cry3Aa3 (Accession #Y00420), Cry3Aa4 (Accession #M30503), Cry3Aa5 (Accession #M37207), Cry3Aa6 (Accession #U10985), Cry3Aa7 (Accession #AJ237900), Cry3Aa8 (Accession #AAS79487), Cry3Aa9 (Accession #AAWO5659), Cry3Aa10 (Accession #AAU29411), Cry3Aa11 (Accession #AY882576), Cry3Aa12 (Accession #ABY49136), Cry3Ba1 (Accession #X17123), Cry3Ba2 (Accession #A07234), Cry3Bb1 (Accession #M89794), Cry3Bb2 (Accession #U31633), Cry3Bb3 (Accession #I15475), Cry3Ca1 (Accession #X59797), Cry4Aa1 (Accession #Y00423), Cry4Aa2 (Accession #D00248), Cry4Aa3 (Accession #AL731825), Cry4A-like (Accession #DQ078744), Cry4Ba1 (Accession #X07423), Cry4Ba2 (Accession #X07082), Cry4Ba3 (Accession #M20242), Cry4Ba4 (Accession #D00247), Cry4Ba5 (Accession #AL731825), Cry4Ba-like (Accession #ABC47686), Cry4Ca1 (Accession #EU646202), Cry5Aa1 (Accession #L07025), Cry5Ab1 (Accession #L07026), Cry5Ac1 (Accession #I34543), Cry5Ad1 (Accession #EF219060), Cry5Ba1 (Accession #U19725), Cry5Ba2 (Accession #EU121522), Cry6Aa1 (Accession #L07022), Cry6Aa2 (Accession #AF499736), Cry6Aa3 (Accession #DQ835612), Cry6Ba1 (Accession #L07024), Cry7Aa1 (Accession #M64478), Cry7Ab1 (Accession #U04367), Cry7Ab2 (Accession #U04368), Cry7Ab3 (Accession #BI 1015188), Cry7Ab4 (Accession #EU380678), Cry7Ab5 (Accession #ABX9555), Cry7Ab6 (Accession #FJ194973), Cry7Ba1 (Accession #ABB70817), Cry7Ca1 (Accession #EF486523), Cry8Aa1 (Accession #U04364), Cry8Ab1 (Accession #EU044830), Cry8Ba1 (Accession #U04365), Cry8Bb1 (Accession #AX543924), Cry8Bc1 (Accession #AX543926), Cry8Ca1 (Accession #U04366), Cry8Ca2 (Accession #AAR98783), Cry8Ca3 (Accession #EU625349), Cry8Da1 (Accession #AB089299), Cry8Da2 (Accession #BD133574), Cry8Da3 (Accession #BD133575), Cry8 Db1 (Accession #AB303980), Cry8Ea1 (Accession #AY329081), Cry8Ea2 (Accession #EU047597), Cry8Fa1 (Accession #AY551093), Cry8Ga1 (Accession #AY590188), Cry8Ga2 (Accession #DQ318860), Cry8Ga3 (Accession #FJ198072), Cry8Ha1 (Accession #EF465532), Cry81a1 (Accession #EU381044), Cry8Ja1 (Accession #EU625348), Cry8 like (Accession #ABS53003), Cry9Aa1 (Accession #X58120), Cry9Aa2 (Accession #X58534), Cry9Aa like (Accession #AAQ52376), Cry9Ba1 (Accession #X75019), Cry9Bb1 (Accession #AY758316), Cry9Ca1 (Accession #Z37527), Cry9Ca2 (Accession #AAQ52375), Cry9Da1 (Accession #D85560), Cry9Da2 (Accession #AF042733), Cry9 Db1 (Accession #AY971349), Cry9Ea1 (Accession #AB011496), Cry9Ea2 (Accession #AF358863), Cry9Ea3 (Accession #EF157307), Cry9Ea4 (Accession #EU760456), Cry9Ea5 (Accession #EU789519), Cry9Ea6 (Accession #EU887516), Cry9Eb1 (Accession #AX189653), Cry9Ec1 (Accession #AF093107), Cry9Ed1 (Accession #AY973867), Cry9 like (Accession #AF093107), Cry10Aa1 (Accession #M12662), Cry10Aa2 (Accession #E00614), Cry10Aa3 (Accession #AL731825), Cry10A like (Accession #DQ167578), Cry1IAa1 (Accession #M31737), Cry1IAa2 (Accession #M22860), Cry1IAa3 (Accession #AL731825), Cry1IAa-like (Accession #DQ166531), Cry11Ba1 (Accession #X86902), Cry11Bb1 (Accession #AF017416), Cry12Aa1 (Accession #L07027), Cry13Aa1 (Accession #L07023), Cry14Aa1 (Accession #U13955), Cry15Aa1 (Accession #M76442), Cry16Aa1 (Accession #X94146), Cry17Aa1 (Accession #X99478), Cry18Aa1 (Accession #X99049), Cry18Ba1 (Accession #AF169250), Cry18Ca1 (Accession #AF169251), Cry19Aa1 (Accession #Y07603), Cry19Ba1 (Accession #D88381), Cry20Aa1 (Accession #U82518), Cry21Aa1 (Accession #I32932), Cry21Aa2 (Accession #I66477), Cry21Ba1 (Accession #AB088406), Cry22Aa1 (Accession #134547), Cry22Aa2 (Accession #AX472772), Cry22Aa3 (Accession #EU715020), Cry22Ab1 (Accession #AAK50456), Cry22Ab2 (Accession #AX472764), Cry22Ba1 (Accession #AX472770), Cry23Aa1 (Accession #AAF76375), Cry24Aa1 (Accession #U88188), Cry24Ba1 (Accession #BAD32657), Cry24Ca1 (Accession #AM158318), Cry25Aa1 (Accession #U88189), Cry26Aa1 (Accession #AF122897), Cry27Aa1 (Accession #AB023293), Cry28Aa1 (Accession #AF132928), Cry28Aa2 (Accession #AF285775), Cry29Aa1 (Accession #AJ251977), Cry30Aa1 (Accession #AJ251978), Cry30Ba1 (Accession #BAD00052), Cry30Ca1 (Accession #BAD67157), Cry30Da1 (Accession #EF095955), Cry30 Db1 (Accession #BAE80088), Cry30Ea1 (Accession #EU503140), Cry30Fa1 (Accession #EU751609), Cry30Ga1 (Accession #EU882064), Cry31Aa1 (Accession #AB031065), Cry31Aa2 (Accession #AY081052), Cry31Aa3 (Accession #AB250922), Cry31Aa4 (Accession #AB274826), Cry31Aa5 (Accession #AB274827), Cry31Ab1 (Accession #AB250923), Cry31Ab2 (Accession #AB274825), Cry31Ac1 (Accession #AB276125), Cry32Aa1 (Accession #AY008143), Cry32Ba1 (Accession #BAB78601), Cry32Ca1 (Accession #BAB78602), Cry32Da1 (Accession #BAB78603), Cry33Aa1 (Accession #AAL26871), Cry34Aa1 (Accession #AAG50341), Cry34Aa2 (Accession #AAK64560), Cry34Aa3 (Accession #AY536899), Cry34Aa4 (Accession #AY536897), Cry34Ab1 (Accession #AAG41671), Cry34Ac1 (Accession #AAG50118), Cry34Ac2 (Accession #AAK64562), Cry34Ac3 (Accession #AY536896), Cry34Ba1 (Accession #AAK64565), Cry34Ba2 (Accession #AY536900), Cry34Ba3 (Accession #AY536898), Cry35Aa1 (Accession #AAG50342), Cry35Aa2 (Accession #AAK64561), Cry35Aa3 (Accession #AY536895), Cry35Aa4 (Accession #AY536892), Cry35Ab1 (Accession #AAG41672), Cry35Ab2 (Accession #AAK64563), Cry35Ab3 (Accession #AY536891), Cry35Ac1 (Accession #AAG50117), Cry35Ba1 (Accession #AAK64566), Cry35Ba2 (Accession #AY536894), Cry35Ba3 (Accession #AY536893), Cry36Aa1 (Accession #AAK64558), Cry37Aa1 (Accession #AAF76376), Cry38Aa1 (Accession #AAK64559), Cry39Aa1 (Accession #BAB72016), Cry40Aa1 (Accession #BAB72018), Cry40Ba1 (Accession #BAC77648), Cry40Ca1 (Accession #EU381045), Cry40Da1 (Accession #EU596478), Cry41Aa1 (Accession #AB116649), Cry41Ab1 (Accession #AB116651), Cry42Aa1 (Accession #AB116652), Cry43Aa1 (Accession #AB115422), Cry43Aa2 (Accession #AB176668), Cry43Ba1 (Accession #AB115422), Cry43-like (Accession #AB115422), Cry44Aa (Accession #BAD08532), Cry45Aa (Accession #BAD22577), Cry46Aa (Accession #BAC79010), Cry46Aa2 (Accession #BAG68906), Cry46Ab (Accession #BAD35170), Cry47Aa (Accession #AY950229), Cry48Aa (Accession #AJ841948), Cry48Aa2 (Accession #AM237205), Cry48Aa3 (Accession #AM237206), Cry48Ab (Accession #AM237207), Cry48Ab2 (Accession #AM237208), Cry49Aa (Accession #AJ841948), Cry49Aa2 (Accession #AM237201), Cry49Aa3 (Accession #AM237203), Cry49Aa4 (Accession #AM237204), Cry49Ab1 (Accession #AM237202), Cry50Aa1 (Accession #AB253419), Cry51Aa1 (Accession #DQ836184), Cry52Aa1 (Accession #EF613489), Cry53Aa1 (Accession #EF633476), Cry54Aa1 (Accession #EU339367), Cry55Aa1 (Accession #EU121521), Cry55Aa2 (Accession #AAE33526).

Examples of delta-endotoxins also include but are not limited to Cry1A proteins of U.S. Pat. Nos. 5,880,275 and 7,858,849; a DIG-3 or DIG-11 toxin (N-terminal deletion of alpha-helix 1 and/or alpha-helix 2 variants of Cry proteins such as Cry1A) of U.S. Pat. Nos. 8,304,604 and 8,304,605, Cry1B of U.S. patent application Ser. No. 10/525,318; Cry1C of U.S. Pat. No. 6,033,874; Cry1F of U.S. Pat. Nos. 5,188,960, 6,218,188; Cry1A/F chimeras of U.S. Pat. Nos. 7,070,982; 6,962,705 and 6,713,063); a Cry2 protein such as Cry2Ab protein of U.S. Pat. No. 7,064,249); a Cry3A protein including but not limited to an engineered hybrid insecticidal protein (eHIP) created by fusing unique combinations of variable regions and conserved blocks of at least two different Cry proteins (US Patent Application Publication Number 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,378,499 and 7,462,760; a Cry9 protein such as such as members of the Cry9A, Cry9B, Cry9C, Cry9D, Cry9E, and Cry9F families; a Cry15 protein of Naimov, et al., (2008) Applied and Environmental Microbiology 74:7145-7151; a Cry22, a Cry34Ab1 protein of U.S. Pat. Nos. 6,127,180, 6,624,145 and 6,340,593; a CryET33 and CryET34 protein of U.S. Pat. Nos. 6,248,535, 6,326,351, 6,399,330, 6,949,626, 7,385,107 and 7,504,229; a CryET33 and CryET34 homologs of US Patent Publication Number 2006/0191034, 2012/0278954, and PCT Publication Number WO 2012/139004; a Cry35Ab1 protein of U.S. Pat. Nos. 6,083,499, 6,548,291 and 6,340,593; a Cry46 protein, a Cry 51 protein, a Cry binary toxin; a TIC901 or related toxin; TIC807 of US 2008/0295207; ET29, ET37, TIC809, TIC810, TIC812, TIC127, TIC128 of PCT US 2006/033867; AXMI-027, AXMI-036, and AXMI-038 of U.S. Pat. No. 8,236,757; AXMI-031, AXMI-039, AXMI-040, AXMI-049 of U.S. Pat. No. 7,923,602; AXMI-018, AXMI-020, and AXMI-021 of WO 2006/083891; AXMI-010 of WO 2005/038032; AXMI-003 of WO 2005/021585; AXMI-008 of US 2004/0250311; AXMI-006 of US 2004/0216186; AXMI-007 of US 2004/0210965; AXMI-009 of US 2004/0210964; AXMI-014 of US 2004/0197917; AXMI-004 of US 2004/0197916; AXMI-028 and AXMI-029 of WO 2006/119457; AXMI-007, AXMI-008, AXMI-0080rf2, AXMI-009, AXMI-014 and AXMI-004 of WO 2004/074462; AXMI-150 of U.S. Pat. No. 8,084,416; AXMI-205 of US20110023184; AXMI-011, AXMI-012, AXMI-013, AXMI-015, AXMI-019, AXMI-044, AXMI-037, AXMI-043, AXMI-033, AXMI-034, AXMI-022, AXMI-023, AXMI-041, AXMI-063, and AXMI-064 of US 2011/0263488; AXMI-R1 and related proteins of US 2010/0197592; AXMI221Z, AXMI222z, AXMI223z, AXMI224z and AXMI225z of WO 2011/103248; AXMI218, AXMI219, AXMI220, AXMI226, AXMI227, AXMI228, AXMI229, AXMI230, and AXMI231 of WO11/103,247; AXMI-115, AXMI-113, AXMI-005, AXMI-163 and AXMI-184 of U.S. Pat. No. 8,334,431; AXMI-001, AXMI-002, AXMI-030, AXMI-035, and AXMI-045 of US 2010/0298211; AXMI-066 and AXMI-076 of US20090144852; AXMI128, AXMI130, AXMI131, AXMI133, AXMI140, AXMI141, AXMI142, AXMI143, AXMI144, AXMI146, AXMI148, AXMI149, AXMI152, AXMI153, AXMI154, AXMI155, AXMI156, AXMI157, AXMI158, AXMI162, AXMI165, AXMI166, AXMI167, AXMI168, AXMI169, AXMI170, AXMI171, AXMI172, AXMI173, AXMI174, AXMI175, AXMI176, AXMI177, AXMI178, AXMI179, AXMI180, AXMI181, AXMI182, AXMI185, AXMI186, AXMI187, AXMI188, AXMI189 of U.S. Pat. No. 8,318,900; AXMI079, AXMI080, AXMI081, AXMI082, AXMI091, AXMI092, AXMI096, AXMI097, AXMI098, AXMI099, AXMI100, AXMI101, AXMI102, AXMI103, AXMI104, AXMI107, AXMI108, AXMI109, AXMI110, AXMI111, AXMI112, AXMI114, AXMI116, AXMI117, AXMI118, AXMI119, AXMI120, AXMI121, AXMI122, AXMI123, AXMI124, AXMI1257, AXMI1268, AXMI127, AXMI129, AXMI164, AXMI151, AXMI161, AXMI183, AXMI132, AXMI138, AXMI137 of US 2010/0005543; Cry proteins such as Cry1A and Cry3A having modified proteolytic sites of U.S. Pat. No. 8,319,019; and a Cry1Ac, Cry2Aa and Cry1Ca toxin protein from Bacillus thuringiensis strain VBTS 2528 of US Patent Application Publication Number 2011/0064710. Other Cry proteins are well known to one skilled in the art (see, Crickmore, et al., “Bacillus thuringiensis toxin nomenclature” (2011), at lifesci.sussex.ac.uk/home/Neil_Crickmore/Bt/which can be accessed on the world-wide web using the “www” prefix). The insecticidal activity of Cry proteins is well known to one skilled in the art (for review, see, van Frannkenhuyzen, (2009) J. Invert. Path. 101:1-16). The use of Cry proteins as transgenic plant traits is well known to one skilled in the art and Cry-transgenic plants including but not limited to Cry1Ac, Cry1Ac+Cry2Ab, Cry1Ab, Cry1A. 105, Cry1F, Cry1Fa2, Cry1F+Cry1Ac, Cry2Ab, Cry3A, mCry3A, Cry3Bb1, Cry34Ab1, Cry35Ab1, Vip3A, mCry3A, Cry9c and CBI-Bt have received regulatory approval (see, Sanahuja, (2011) Plant Biotech Journal 9:283-300 and the CERA (2010) GM Crop Database Center for Environmental Risk Assessment (CERA), ILSI Research Foundation, Washington D.C. at cera-gmc.org/index.php?action=gm_crop_database which can be accessed on the world-wide web using the “www” prefix). Pesticidal proteins also include insecticidal lipases including lipid acyl hydrolases of U.S. Pat. No. 7,491,869, and cholesterol oxidases such as from Streptomyces (Purcell et al. (1993) Biochem Biophys Res Commun 15:1406-1413). Pesticidal proteins also include VIP (vegetative insecticidal proteins) toxins of U.S. Pat. Nos. 5,877,012, 6,107,279, 6,137,033, 7,244,820, 7,615,686, and 8,237,020, and the like. Other VIP proteins are well known to one skilled in the art (see, lifesci.sussex.ac.uk/home/Neil_Crickmore/Bt/vip.html which can be accessed on the world-wide web using the “www” prefix). Pesticidal proteins also include toxin complex (TC) proteins, obtainable from organisms such as Xenorhabdus, Photorhabdus and Paenibacillus (see, U.S. Pat. Nos. 7,491,698 and 8,084,418). Some TC proteins have “stand alone” insecticidal activity and other TC proteins enhance the activity of the stand-alone toxins produced by the same given organism. The toxicity of a “stand-alone” TC protein (from Photorhabdus, Xenorhabdus or Paenibacillus, for example) can be enhanced by one or more TC protein “potentiators” derived from a source organism of a different genus. There are three main types of TC proteins. As referred to herein, Class A proteins (“Protein A”) are stand-alone toxins. Class B proteins (“Protein B”) and Class C proteins (“Protein C”) enhance the toxicity of Class A proteins. Examples of Class A proteins are TcbA, TcdA, XptA1 and XptA2. Examples of Class B proteins are TcaC, TcdB, XptB1Xb and XptC1Wi. Examples of Class C proteins are TccC, XptC1Xb and XptB1Wi. Pesticidal proteins also include spider, snake and scorpion venom proteins. Examples of spider venom peptides include but are not limited to lycotoxin-1 peptides and mutants thereof (U.S. Pat. No. 8,334,366).

(C) A polynucleotide encoding an insect-specific hormone or pheromone such as an ecdysteroid and juvenile hormone, a variant thereof, a mimetic based thereon or an antagonist or agonist thereof. See, for example, the disclosure by Hammock, et al., (1990) Nature 344:458, of baculovirus expression of cloned juvenile hormone esterase, an inactivator of juvenile hormone.

(D) A polynucleotide encoding an insect-specific peptide which, upon expression, disrupts the physiology of the affected pest. For example, see the disclosures of, Regan, (1994) J. Biol. Chem. 269:9 (expression cloning yields DNA coding for insect diuretic hormone receptor); Pratt, et al., (1989) Biochem. Biophys. Res. Comm. 163:1243 (an allostatin is identified in Diploptera puntata); Chattopadhyay, et al., (2004) Critical Reviews in Microbiology 30(1):33-54; Zjawiony, (2004) J Nat Prod 67(2):300-310; Carlini and Grossi-de-Sa, (2002) Toxicon 40(11):1515-1539; Ussuf, et al., (2001) Curr Sci. 80(7):847-853 and Vasconcelos and Oliveira, (2004) Toxicon 44(4):385-403. See also, U.S. Pat. No. 5,266,317 to Tomalski, et al., who disclose genes encoding insect-specific toxins.

(E) A polynucleotide encoding an enzyme responsible for a hyperaccumulation of a monoterpene, a sesquiterpene, a steroid, hydroxamic acid, a phenylpropanoid derivative or another non-protein molecule with insecticidal activity.

(F) A polynucleotide encoding an enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See, PCT Application WO 1993/02197 in the name of Scott, et al., which discloses the nucleotide sequence of a callase gene. DNA molecules which contain chitinase-encoding sequences can be obtained, for example, from the ATCC under Accession Numbers 39637 and 67152. See also, Kramer, et al., (1993) Insect Biochem. Molec. Biol. 23:691, who teach the nucleotide sequence of a cDNA encoding tobacco hookworm chitinase and Kawalleck, et al., (1993) Plant Molec. Biol. 21:673, who provide the nucleotide sequence of the parsley ubi4-2 polyubiquitin gene, and U.S. Pat. Nos. 6,563,020; 7,145,060 and 7,087,810.

(G) A polynucleotide encoding a molecule that stimulates signal transduction. For example, see the disclosure by Botella, et al., (1994) Plant Molec. Biol. 24:757, of nucleotide sequences for mung bean calmodulin cDNA clones, and Griess, et al., (1994) Plant Physiol. 104:1467, who provide the nucleotide sequence of a maize calmodulin cDNA clone.

(H) A polynucleotide encoding a hydrophobic moment peptide. See, PCT Application WO 1995/16776 and U.S. Pat. No. 5,580,852 disclosure of peptide derivatives of Tachyplesin which inhibit fungal plant pathogens) and PCT Application WO 1995/18855 and U.S. Pat. No. 5,607,914 (teaches synthetic antimicrobial peptides that confer disease resistance).

(I) A polynucleotide encoding a membrane permease, a channel former or a channel blocker. For example, see the disclosure by Jaynes, et al., (1993) Plant Sci. 89:43, of heterologous expression of a cecropin-beta lytic peptide analog to render transgenic tobacco plants resistant to Pseudomonas solanacearum.

(J) A gene encoding a viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See, Beachy, et al., (1990) Ann. Rev. Phytopathol. 28:451. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

(K) A gene encoding an insect-specific antibody or an immunotoxin derived therefrom. Thus, an antibody targeted to a critical metabolic function in the insect gut would inactivate an affected enzyme, killing the insect. Cf. Taylor, et al., Abstract #497, SEVENTH INT'L SYMPOSIUM ON MOLECULAR PLANT-MICROBE INTERACTIONS (Edinburgh, Scotland, 1994) (enzymatic inactivation in transgenic tobacco via production of single-chain antibody fragments).

(L) A gene encoding a virus-specific antibody. See, for example, Tavladoraki, et al., (1993) Nature 366:469, who show that transgenic plants expressing recombinant antibody genes are protected from virus attack.

(M) A polynucleotide encoding a developmental-arrestive protein produced in nature by a pathogen or a parasite. Thus, fungal endo alpha-1,4-D-polygalacturonases facilitate fungal colonization and plant nutrient release by solubilizing plant cell wall homo-alpha-1,4-D-galacturonase. See, Lamb, et al., (1992) Bio/Technology 10:1436. The cloning and characterization of a gene which encodes a bean endopolygalacturonase-inhibiting protein is described by Toubart, et al., (1992) Plant J. 2:367.

(N) A polynucleotide encoding a developmental-arrestive protein produced in nature by a plant. For example, Logemann, et al., (1992) Bio/Technology 10:305, have shown that transgenic plants expressing the barley ribosome-inactivating gene have an increased resistance to fungal disease.

(O) Genes involved in the Systemic Acquired Resistance (SAR) Response and/or the pathogenesis related genes. Briggs, (1995) Current Biology 5(2), Pieterse and Van Loon, (2004) Curr. Opin. Plant Bio. 7(4):456-64 and Somssich, (2003) Cell 113(7):815-6.

(P) Antifungal genes (Cornelissen and Melchers, (1993) Pl. Physiol. 101:709-712 and Parijs, et al., (1991) Planta 183:258-264 and Bushnell, et al., (1998) Can. J. of Plant Path. 20(2):137-149. Also see, U.S. patent application Ser. Nos. 09/950,933; 11/619,645; 11/657,710; 11/748,994; 11/774,121 and U.S. Pat. Nos. 6,891,085 and 7,306,946. LysM Receptor-like kinases for the perception of chitin fragments as a first step in plant defense response against fungal pathogens (US 2012/0110696).

(Q) Detoxification genes, such as for fumonisin, beauvericin, moniliformin and zearalenone and their structurally related derivatives. For example, see, U.S. Pat. Nos. 5,716,820; 5,792,931; 5,798,255; 5,846,812; 6,083,736; 6,538,177; 6,388,171 and 6,812,380.

(R) A polynucleotide encoding a Cystatin and cysteine proteinase inhibitors. See, U.S. Pat. No. 7,205,453.

(S) Defensin genes. See, WO 2003/000863 and U.S. Pat. Nos. 6,911,577; 6,855,865; 6,777,592 and 7,238,781.

(T) Genes conferring resistance to nematodes. See, e.g., PCT Application WO 1996/30517; PCT Application WO 1993/19181, WO 2003/033651 and Urwin, et al., (1998) Planta 204:472-479, Williamson, (1999) Curr Opin Plant Bio. 2(4):327-31; U.S. Pat. Nos. 6,284,948 and 7,301,069 and miR164 genes (WO 2012/058266).

(U) Genes that confer resistance to Phytophthora Root Rot, such as the Rps 1, Rps 1-a, Rps 1-b, Rps 1-c, Rps 1-d, Rps 1-e, Rps 1-k, Rps 2, Rps 3-a, Rps 3-b, Rps 3-c, Rps 4, Rps 5, Rps 6, Rps 7 and other Rps genes. See, for example, Shoemaker, et al., Phytophthora Root Rot Resistance Gene Mapping in Soybean, Plant Genome IV Conference, San Diego, Calif. (1995).

(V) Genes that confer resistance to Brown Stem Rot, such as described in U.S. Pat. No. 5,689,035 and incorporated by reference for this purpose.

(W) Genes that confer resistance to Colletotrichum, such as described in US Patent Application Publication US 2009/0035765 and incorporated by reference for this purpose. This includes the Rcg locus that may be utilized as a single locus conversion.

ii. Transgenes that Confer Resistance to a Herbicide.

(A) A polynucleotide encoding resistance to a herbicide that inhibits the growing point or meristem, such as an imidazolinone or a sulfonylurea. Exemplary genes in this category code for mutant ALS and AHAS enzyme as described, for example, by Lee, et al., (1988) EMBO J. 7:1241 and Miki, et al., (1990) Theor. Appl. Genet. 80:449, respectively. See also, U.S. Pat. Nos. 5,605,011; 5,013,659; 5,141,870; 5,767,361; 5,731,180; 5,304,732; 4,761,373; 5,331,107; 5,928,937 and 5,378,824; U.S. patent application Ser. No. 11/683,737 and International Publication WO 1996/33270.

(B) A polynucleotide encoding a protein for resistance to Glyphosate (resistance imparted by mutant 5-enolpyruvl-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes), and pyridinoxy or phenoxy proprionic acids and cyclohexones (ACCase inhibitor-encoding genes). See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes. See also, U.S. Pat. Nos. 6,566,587; 6,338,961; 6,248,876 B1; 6,040,497; 5,804,425; 5,633,435; 5,145,783; 4,971,908; 5,312,910; 5,188,642; 5,094,945, 4,940,835; 5,866,775; 6,225,114 B1; 6,130,366; 5,310,667; 4,535,060; 4,769,061; 5,633,448; 5,510,471; Re. 36,449; RE 37,287 E and 5,491,288 and International Publications EP 1173580; WO 2001/66704; EP 1173581 and EP 1173582, which are incorporated herein by reference for this purpose.

Glyphosate resistance is also imparted to plants that express a gene encoding a glyphosate oxido-reductase enzyme as described more fully in U.S. Pat. Nos. 5,776,760 and 5,463,175, which are incorporated herein by reference for this purpose. In addition glyphosate resistance can be imparted to plants by the over expression of genes encoding glyphosate N-acetyltransferase. See, for example, U.S. Pat. Nos. 7,462,481; 7,405,074 and US Patent Application Publication Number US 2008/0234130. A DNA molecule encoding a mutant aroA gene can be obtained under ATCC Accession Number 39256, and the nucleotide sequence of the mutant gene is disclosed in U.S. Pat. No. 4,769,061 to Comai. EP Application Number 0 333 033 to Kumada, et al., and U.S. Pat. No. 4,975,374 to Goodman, et al., disclose nucleotide sequences of glutamine synthetase genes which confer resistance to herbicides such as L-phosphinothricin. The nucleotide sequence of a phosphinothricin-acetyl-transferase gene is provided in EP Application Numbers 0 242 246 and 0 242 236 to Leemans, et al.; De Greef, et al., (1989) Bio/Technology 7:61, describe the production of transgenic plants that express chimeric bar genes coding for phosphinothricin acetyl transferase activity. See also, U.S. Pat. Nos. 5,969,213; 5,489,520; 5,550,318; 5,874,265; 5,919,675; 5,561,236; 5,648,477; 5,646,024; 6,177,616 B1 and 5,879,903, which are incorporated herein by reference for this purpose. Exemplary genes conferring resistance to phenoxy proprionic acids and cyclohexones, such as sethoxydim and haloxyfop, are the Acc1-S1, Acc1-S2 and Acc1-S3 genes described by Marshall, et al., (1992) Theor. Appl. Genet. 83:435.

(C) A polynucleotide encoding a protein for resistance to herbicide that inhibits photosynthesis, such as a triazine (psbA and gs+ genes) and a benzonitrile (nitrilase gene). Przibilla, et al., (1991) Plant Cell 3:169, describe the transformation of Chlamydomonas with plasmids encoding mutant psbA genes. Nucleotide sequences for nitrilase genes are disclosed in U.S. Pat. No. 4,810,648 to Stalker and DNA molecules containing these genes are available under ATCC Accession Numbers 53435, 67441 and 67442. Cloning and expression of DNA coding for a glutathione S-transferase is described by Hayes, et al., (1992) Biochem. J. 285:173.

(D) A polynucleotide encoding a protein for resistance to Acetohydroxy acid synthase, which has been found to make plants that express this enzyme resistant to multiple types of herbicides, has been introduced into a variety of plants (see, e.g., Hattori, et al., (1995) Mol Gen Genet. 246:419). Other genes that confer resistance to herbicides include: a gene encoding a chimeric protein of rat cytochrome P4507A1 and yeast NADPH-cytochrome P450 oxidoreductase (Shiota, et al., (1994) Plant Physiol 106:17), genes for glutathione reductase and superoxide dismutase (Aono, et al., (1995) Plant Cell Physiol 36:1687) and genes for various phosphotransferases (Datta, et al., (1992) Plant Mol Biol 20:619).

(E) A polynucleotide encoding resistance to a herbicide targeting Protoporphyrinogen oxidase (protox) which is necessary for the production of chlorophyll. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306 B1; 6,282,837 B1 and 5,767,373 and International Publication WO 2001/12825.

(F) The aad-1 gene (originally from Sphingobium herbicidovorans) encodes the aryloxyalkanoate dioxygenase (AAD-1) protein. The trait confers tolerance to 2,4-dichlorophenoxyacetic acid and aryloxyphenoxypropionate (commonly referred to as “fop” herbicides such as quizalofop) herbicides. The aad-1 gene, itself, for herbicide tolerance in plants was first disclosed in WO 2005/107437 (see also, US 2009/0093366). The aad-12 gene, derived from Delftia acidovorans, which encodes the aryloxyalkanoate dioxygenase (AAD-12) protein that confers tolerance to 2,4-dichlorophenoxyacetic acid and pyridyloxyacetate herbicides by deactivating several herbicides with an aryloxyalkanoate moiety, including phenoxy auxin (e.g., 2,4-D, MCPA), as well as pyridyloxy auxins (e.g., fluoroxypyr, triclopyr).

(G) A polynucleotide encoding a herbicide resistant dicamba monooxygenase disclosed in US Patent Application Publication 2003/0135879 for imparting dicamba tolerance.

(H) A polynucleotide molecule encoding bromoxynil nitrilase (Bxn) disclosed in U.S. Pat. No. 4,810,648 for imparting bromoxynil tolerance.

(I) A polynucleotide molecule encoding phytoene (crtl) described in Misawa, et al., (1993) Plant J. 4:833-840 and in Misawa, et al., (1994) Plant J. 6:481-489 for norflurazon tolerance.

iii. Transgenes that Confer or Contribute to an Altered Grain Characteristic

(A) Altered fatty acids, for example, by (1) Down-regulation of stearoyl-ACP to increase stearic acid content of the plant. See, Knultzon, et al., (1992) Proc. Natl. Acad. Sci. USA 89:2624 and WO 1999/64579 (Genes to Alter Lipid Profiles in Corn); (2) Elevating oleic acid via FAD-2 gene modification and/or decreasing linolenic acid via FAD-3 gene modification (see, U.S. Pat. Nos. 6,063,947; 6,323,392; 6,372,965 and WO 1993/11245); (3) Altering conjugated linolenic or linoleic acid content, such as in WO 2001/12800; (4) Altering LEC1, AGP, Dek1, Superal1, mil ps, various Ipa genes such as Ipa1, Ipa3, hpt or hggt. For example, see, WO 2002/42424, WO 1998/22604, WO 2003/011015, WO 2002/057439, WO 2003/011015, U.S. Pat. Nos. 6,423,886, 6,197,561, 6,825,397 and US Patent Application Publication Numbers US 2003/0079247, US 2003/0204870 and Rivera-Madrid, et al., (1995) Proc. Natl. Acad. Sci. 92:5620-5624; (5) Genes encoding delta-8 desaturase for making long-chain polyunsaturated fatty acids (U.S. Pat. Nos. 8,058,571 and 8,338,152), delta-9 desaturase for lowering saturated fats (U.S. Pat. No. 8,063,269), Primula .DELTA.6-desaturase for improving omega-3 fatty acid profiles; (6) Isolated nucleic acids and proteins associated with lipid and sugar metabolism regulation, in particular, lipid metabolism protein (LMP) used in methods of producing transgenic plants and modulating levels of seed storage compounds including lipids, fatty acids, starches or seed storage proteins and use in methods of modulating the seed size, seed number, seed weights, root length and leaf size of plants (EP 2404499); (7) Altering expression of a High-Level Expression of Sugar-Inducible 2 (HSI2) protein in the plant to increase or decrease expression of HSI2 in the plant. Increasing expression of HSI2 increases oil content while decreasing expression of HSI2 decreases abscisic acid sensitivity and/or increases drought resistance (US Patent Application Publication Number 2012/0066794); (8) Expression of cytochrome b5 (Cb5) alone or with FAD2 to modulate oil content in plant seed, particularly to increase the levels of omega-3 fatty acids and improve the ratio of omega-6 to omega-3 fatty acids (US Patent Application Publication Number 2011/0191904); and (9) Nucleic acid molecules encoding wrinkled1-like polypeptides for modulating sugar metabolism (U.S. Pat. No. 8,217,223).

(B) Altered phosphorus content, for example, by the (1) introduction of a phytase-encoding gene would enhance breakdown of phytate, adding more free phosphate to the transformed plant. For example, see, Van Hartingsveldt, et al., (1993) Gene 127:87, for a disclosure of the nucleotide sequence of an Aspergillus niger phytase gene; and (2) modulating a gene that reduces phytate content. In maize, this, for example, could be accomplished, by cloning and then re-introducing DNA associated with one or more of the alleles, such as the LPA alleles, identified in maize mutants characterized by low levels of phytic acid, such as in WO 2005/113778 and/or by altering inositol kinase activity as in WO 2002/059324, US Patent Application Publication Number 2003/0009011, WO 2003/027243, US Patent Application Publication Number 2003/0079247, WO 1999/05298, U.S. Pat. No. 6,197,561, U.S. Pat. No. 6,291,224, U.S. Pat. No. 6,391,348, WO 2002/059324, US Patent Application Publication Number 2003/0079247, WO 1998/45448, WO 1999/55882, WO 2001/04147.

(C) Altered carbohydrates affected, for example, by altering a gene for an enzyme that affects the branching pattern of starch or, a gene altering thioredoxin such as NTR and/or TRX (see, U.S. Pat. No. 6,531,648. which is incorporated by reference for this purpose) and/or a gamma zein knock out or mutant such as cs27 or TUSC27 or en27 (see, U.S. Pat. No. 6,858,778 and US Patent Application Publication Number 2005/0160488, US Patent Application Publication Number 2005/0204418, which are incorporated by reference for this purpose). See, Shiroza, et al., (1988) J. Bacteriol. 170:810 (nucleotide sequence of Streptococcus mutant fructosyltransferase gene), Steinmetz, et al., (1985) Mol. Gen. Genet. 200:220 (nucleotide sequence of Bacillus subtilis levansucrase gene), Pen, et al., (1992) Bio/Technology 10:292 (production of transgenic plants that express Bacillus licheniformis alpha-amylase), Elliot, et al., (1993) Plant Molec. Biol. 21:515 (nucleotide sequences of tomato invertase genes), Sogaard, et al., (1993) J. Biol. Chem. 268:22480 (site-directed mutagenesis of barley alpha-amylase gene) and Fisher, et al., (1993) Plant Physiol. 102:1045 (maize endosperm starch branching enzyme II), WO 1999/10498 (improved digestibility and/or starch extraction through modification of UDP-D-xylose 4-epimerase, Fragile 1 and 2, Ref1, HCHL, C4H), U.S. Pat. No. 6,232,529 (method of producing high oil seed by modification of starch levels (AGP)). The fatty acid modification genes mentioned herein may also be used to affect starch content and/or composition through the interrelationship of the starch and oil pathways.

(D) Altered antioxidant content or composition, such as alteration of tocopherol or tocotrienols. For example, see, U.S. Pat. No. 6,787,683, US Patent Application Publication Number 2004/0034886 and WO 2000/68393 involving the manipulation of antioxidant levels and WO 2003/082899 through alteration of a homogentisate geranyl geranyl transferase (hggt).

(E) Altered essential seed amino acids. For example, see, U.S. Pat. No. 6,127,600 (method of increasing accumulation of essential amino acids in seeds), U.S. Pat. No. 6,080,913 (binary methods of increasing accumulation of essential amino acids in seeds), U.S. Pat. No. 5,990,389 (high lysine), WO 1999/40209 (alteration of amino acid compositions in seeds), WO 1999/29882 (methods for altering amino acid content of proteins), U.S. Pat. No. 5,850,016 (alteration of amino acid compositions in seeds), WO 1998/20133 (proteins with enhanced levels of essential amino acids), U.S. Pat. No. 5,885,802 (high methionine), U.S. Pat. No. 5,885,801 (high threonine), U.S. Pat. No. 6,664,445 (plant amino acid biosynthetic enzymes), U.S. Pat. No. 6,459,019 (increased lysine and threonine), U.S. Pat. No. 6,441,274 (plant tryptophan synthase beta subunit), U.S. Pat. No. 6,346,403 (methionine metabolic enzymes), U.S. Pat. No. 5,939,599 (high sulfur), U.S. Pat. No. 5,912,414 (increased methionine), WO 1998/56935 (plant amino acid biosynthetic enzymes), WO 1998/45458 (engineered seed protein having higher percentage of essential amino acids), WO 1998/42831 (increased lysine), U.S. Pat. No. 5,633,436 (increasing sulfur amino acid content), U.S. Pat. No. 5,559,223 (synthetic storage proteins with defined structure containing programmable levels of essential amino acids for improvement of the nutritional value of plants), WO 1996/01905 (increased threonine), WO 1995/15392 (increased lysine), US Patent Application Publication Number 2003/0163838, US Patent Application Publication Number 2003/0150014, US Patent Application Publication Number 2004/0068767, U.S. Pat. No. 6,803,498, WO 2001/79516.

iv. Genes that Control Male-Sterility

There are several methods of conferring genetic male sterility available, such as multiple mutant genes at separate locations within the genome that confer male sterility, as disclosed in U.S. Pat. Nos. 4,654,465 and 4,727,219 to Brar, et al., and chromosomal translocations as described by Patterson in U.S. Pat. Nos. 3,861,709 and 3,710,511. In addition to these methods, Albertsen, et al., U.S. Pat. No. 5,432,068, describe a system of nuclear male sterility which includes: identifying a gene which is critical to male fertility; silencing this native gene which is critical to male fertility; removing the native promoter from the essential male fertility gene and replacing it with an inducible promoter; inserting this genetically engineered gene back into the plant; and thus creating a plant that is male sterile because the inducible promoter is not “on” resulting in the male fertility gene not being transcribed. Fertility is restored by inducing or turning “on”, the promoter, which in turn allows the gene that confers male fertility to be transcribed. Non-limiting examples include: (A) Introduction of a deacetylase gene under the control of a tapetum-specific promoter and with the application of the chemical N—Ac-PPT (WO 2001/29237); (B) Introduction of various stamen-specific promoters (WO 1992/13956, WO 1992/13957); and (C) Introduction of the barnase and the barstar gene (Paul, et al., (1992) Plant Mol. Biol. 19:611-622). For additional examples of nuclear male and female sterility systems and genes, see also, U.S. Pat. Nos. 5,859,341; 6,297,426; 5,478,369; 5,824,524; 5,850,014 and 6,265,640, all of which are hereby incorporated by reference.

v. Genes that Create a Site for Site Specific DNA Integration.

This includes the introduction of FRT sites that may be used in the FLP/FRT system and/or Lox sites that may be used in the Cre/Loxp system. For example, see, Lyznik, et al., (2003) Plant Cell Rep 21:925-932 and WO 1999/25821, which are hereby incorporated by reference. Other systems that may be used include the Gln recombinase of phage Mu (Maeser, et al., (1991) Vicki Chandler, The Maize Handbook ch. 118 (Springer-Verlag 1994), the Pin recombinase of E. coli (Enomoto, et al., 1983) and the R/RS system of the pSRi plasmid (Araki, et al., 1992).

vi. Genes that Affect Abiotic Stress Resistance

Including but not limited to flowering, ear and seed development, enhancement of nitrogen utilization efficiency, altered nitrogen responsiveness, drought resistance or tolerance, cold resistance or tolerance and salt resistance or tolerance and increased yield under stress. Non-limiting examples include: (A) For example, see: WO 2000/73475 where water use efficiency is altered through alteration of malate; U.S. Pat. Nos. 5,892,009, 5,965,705, 5,929,305, 5,891,859, 6,417,428, 6,664,446, 6,706,866, 6,717,034, 6,801,104, WO 2000/060089, WO 2001/026459, WO 2001/035725, WO 2001/034726, WO 2001/035727, WO 2001/036444, WO 2001/036597, WO 2001/036598, WO 2002/015675, WO 2002/017430, WO 2002/077185, WO 2002/079403, WO 2003/013227, WO 2003/013228, WO 2003/014327, WO 2004/031349, WO 2004/076638, WO 199809521; (B) WO 199938977 describing genes, including CBF genes and transcription factors effective in mitigating the negative effects of freezing, high salinity and drought on plants, as well as conferring other positive effects on plant phenotype; (C) US Patent Application Publication Number 2004/0148654 and WO 2001/36596 where abscisic acid is altered in plants resulting in improved plant phenotype such as increased yield and/or increased tolerance to abiotic stress; (D) WO 2000/006341, WO 2004/090143, U.S. Pat. Nos. 7,531,723 and 6,992,237 where cytokinin expression is modified resulting in plants with increased stress tolerance, such as drought tolerance, and/or increased yield. Also see, WO 2002/02776, WO 2003/052063, JP 2002/281975, U.S. Pat. No. 6,084,153, WO 2001/64898, U.S. Pat. No. 6,177,275 and U.S. Pat. No. 6,107,547 (enhancement of nitrogen utilization and altered nitrogen responsiveness); (E) For ethylene alteration, see, US Patent Application Publication Number 2004/0128719, US Patent Application Publication Number 2003/0166197 and WO 2000/32761; (F) For plant transcription factors or transcriptional regulators of abiotic stress, see, e.g., US Patent Application Publication Number 2004/0098764 or US Patent Application Publication Number 2004/0078852; (G) Genes that increase expression of vacuolar pyrophosphatase such as AVP1 (U.S. Pat. No. 8,058,515) for increased yield; nucleic acid encoding a HSFA4 or a HSFA5 (Heat Shock Factor of the class A4 or A5) polypeptides, an oligopeptide transporter protein (OPT4-like) polypeptide; a plastochron2-like (PLA2-like) polypeptide or a Wuschel related homeobox 1-like (WOX1-like) polypeptide (U. Patent Application Publication Number US 2011/0283420); (H) Down regulation of polynucleotides encoding poly (ADP-ribose) polymerase (PARP) proteins to modulate programmed cell death (U.S. Pat. No. 8,058,510) for increased vigor; (I) Polynucleotide encoding DTP21 polypeptides for conferring drought resistance (US Patent Application Publication Number US 2011/0277181); (J) Nucleotide sequences encoding ACC Synthase 3 (ACS3) proteins for modulating development, modulating response to stress, and modulating stress tolerance (US Patent Application Publication Number US 2010/0287669); (K) Polynucleotides that encode proteins that confer a drought tolerance phenotype (DTP) for conferring drought resistance (WO 2012/058528); (L) Tocopherol cyclase (TC) genes for conferring drought and salt tolerance (US Patent Application Publication Number 2012/0272352); (M) CAAX amino terminal family proteins for stress tolerance (U.S. Pat. No. 8,338,661); (N) Mutations in the SAL1 encoding gene have increased stress tolerance, including increased drought resistant (US Patent Application Publication Number 2010/0257633); (O) Expression of a nucleic acid sequence encoding a polypeptide selected from the group consisting of: GRF polypeptide, RAA1-like polypeptide, SYR polypeptide, ARKL polypeptide, and YTP polypeptide increasing yield-related traits (US Patent Application Publication Number 2011/0061133); and (P) Modulating expression in a plant of a nucleic acid encoding a Class III Trehalose Phosphate Phosphatase (TPP) polypeptide for enhancing yield-related traits in plants, particularly increasing seed yield (US Patent Application Publication Number 2010/0024067).

Other genes and transcription factors that affect plant growth and agronomic traits such as yield, flowering, plant growth and/or plant structure, can be introduced or introgressed into plants, see e.g., WO 1997/49811 (LHY), WO 1998/56918 (ESD4), WO 1997/10339 and U.S. Pat. No. 6,573,430 (TFL), U.S. Pat. No. 6,713,663 (FT), WO 1996/14414 (CON), WO 1996/38560, WO 2001/21822 (VRN1), WO 2000/44918 (VRN2), WO 1999/49064 (GI), WO 2000/46358 (FR1), WO 1997/29123, U.S. Pat. No. 6,794,560, U.S. Pat. No. 6,307,126 (GAI), WO 1999/09174 (D8 and Rht) and WO 2004/076638 and WO 2004/031349 (transcription factors).

vii. Genes that Confer Increased Yield

Non-limiting examples of genes that confer increased yield are: (A) A transgenic crop plant transformed by a 1-AminoCyclopropane-1-Carboxylate Deaminase-like Polypeptide (ACCDP) coding nucleic acid, wherein expression of the nucleic acid sequence in the crop plant results in the plant's increased root growth, and/or increased yield, and/or increased tolerance to environmental stress as compared to a wild type variety of the plant (U.S. Pat. No. 8,097,769); (B) Over-expression of maize zinc finger protein gene (Zm-ZFP1) using a seed preferred promoter has been shown to enhance plant growth, increase kernel number and total kernel weight per plant (US Patent Application Publication Number 2012/0079623); (C) Constitutive over-expression of maize lateral organ boundaries (LOB) domain protein (Zm-LOBDP1) has been shown to increase kernel number and total kernel weight per plant (US Patent Application Publication Number 2012/0079622); (D) Enhancing yield-related traits in plants by modulating expression in a plant of a nucleic acid encoding a VIM1 (Variant in Methylation 1)-like polypeptide or a VTC2-like (GDP-L-galactose phosphorylase) polypeptide or a DUF1685 polypeptide or an ARF6-like (Auxin Responsive Factor) polypeptide (WO 2012/038893); (E) Modulating expression in a plant of a nucleic acid encoding a Ste20-like polypeptide or a homologue thereof gives plants having increased yield relative to control plants (EP 2431472); and (F) Genes encoding nucleoside diphosphatase kinase (NDK) polypeptides and homologs thereof for modifying the plant's root architecture (US Patent Application Publication Number 2009/0064373).

### IX. Methods of Use

Methods disclosed herein comprise methods for controlling a pest (i.e., a Coleopteran plant pest, including a Diabrotica plant pest, such as, D. virgifera virgifera, D. barberi, D. virgifera zeae, D. speciosa, or D. undecimpunctata howardi). In one embodiment, the method comprises feeding or applying to a pest a composition comprising a silencing element of the invention, wherein said silencing element, when ingested or contacted by an insect pest (i.e., but not limited to, a Coleopteran plant pest including a Diabrotica plant pest, such as, D. virgifera virgifera, D. barberi, D. virgifera zeae, D. speciosa, or D. undecimpunctata howardi), reduces the level of a target polynucleotide of the pest and thereby controls the pest. The pest can be fed the silencing element in a variety of ways. For example, in an embodiment, the polynucleotide comprising the silencing element is introduced into a plant. As the plant pest feeds on the plant or part thereof expressing these sequences, the silencing element is delivered to the pest. When the silencing element is delivered to the plant in this manner, it is recognized that the silencing element can be expressed constitutively or alternatively, it may be produced in a stage-specific manner by employing the various inducible or tissue-preferred or developmentally regulated promoters that are discussed elsewhere herein. In specific embodiments, the silencing element is expressed in the roots, stalk or stem, leaf including pedicel, xylem and phloem, fruit or reproductive tissue, silk, flowers and all parts therein or any combination thereof.

In another method, a composition comprising at least one silencing element disclosed herein is applied to a plant. In such embodiments, the silencing element can be formulated in an agronomically suitable and/or environmentally acceptable carrier, which is preferably, suitable for dispersal in fields. In addition, the carrier can also include compounds that increase the half life of the composition. In specific embodiments, the composition comprising the silencing element is formulated in such a manner such that it persists in the environment for a length of time sufficient to allow it to be delivered to a pest. In such embodiments, the composition can be applied to an area inhabited by a pest. In one embodiment, the composition is applied externally to a plant (i.e., by spraying a field) to protect the plant from pests.

In certain embodiments, the disclosed polynucleotides or constructs can be stacked with any combination of polynucleotide sequences of interest in order to create plants with a desired trait. A trait, as used herein, refers to the phenotype derived from a particular sequence or groups of sequences. For example, the disclosed polynucleotides may be stacked with any other polynucleotides encoding polypeptides having pesticidal and/or insecticidal activity, such as other Bacillus thuringiensis toxic proteins (described in U.S. Pat. Nos. 5,366,892; 5,747,450; 5,737,514; 5,723,756; 5,593,881; and Geiser et al. (1986) Gene 48:109), lectins (Van Damme et al. (1994) Plant Mol. Biol. 24:825, pentin (described in U.S. Pat. No. 5,981,722), and the like. The combinations generated can also include multiple copies of any one of the polynucleotides of interest. The disclosed polynucleotides can also be stacked with any other gene or combination of genes to produce plants with a variety of desired trait combinations including, but not limited to, traits desirable for animal feed such as high oil genes (e.g., U.S. Pat. No. 6,232,529); balanced amino acids (e.g., hordothionins (U.S. Pat. Nos. 5,990,389; 5,885,801; 5,885,802; and 5,703,409); barley high lysine (Williamson et al. (1987) Eur. J. Biochem. 165:99-106; and WO 98/20122) and high methionine proteins (Pedersen et al. (1986) J. Biol. Chem. 261:6279; Kirihara et al. (1988) Gene 71:359; and Musumura et al. (1989) Plant Mol. Biol. 12:123)); increased digestibility (e.g., modified storage proteins (U.S. application Ser. No. 10/053,410, filed Nov. 7, 2001); and thioredoxins (U.S. application Ser. No. 10/005,429, filed Dec. 3, 2001)); the disclosures of which are herein incorporated by reference.

Disclosed polynucleotides can also be stacked with traits desirable for disease or herbicide resistance (e.g., fumonisin detoxification genes (U.S. Pat. No. 5,792,931); avirulence and disease resistance genes (Jones et al. (1994) Science 266:789; Martin et al. (1993) Science 262:1432; Mindrinos et al. (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)); the disclosures of which are herein incorporated by reference. One could also combine the disclosed polynucleotides with polynucleotides providing agronomic traits such as male sterility (e.g., see U.S. Pat. No. 5,583,210), stalk strength, drought resistance (e.g., U.S. Pat. No. 7,786,353), flowering time, or transformation technology traits such as cell cycle regulation or gene targeting (e.g., WO 99/61619, WO 00/17364, and WO 99/25821); the disclosures of which are herein incorporated by reference.

These stacked combinations can be created by any method including, but not limited to, cross-breeding plants by any conventional or TopCross methodology, or genetic transformation. If the sequences are stacked by genetically transforming the plants (i.e., molecular stacks), the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

The following examples are offered by way of illustration and not by way of limitation.

### EXPERIMENTAL

### Example 1: Sequences Having Insecticidal Activity

Nucleic acid sequences disclosed herein comprise the following nucleic acid sequences. Certain sequences are exemplary and were shown to have insecticidal activity against corn rootworms using the assay methods described in Examples 3 and 4 as set forth below. Such sequences or their complements can be used in the disclosed methods as described herein above and below. Methods for making inhibitory sequences are known in the art. DNA constructs, vectors, transgenic cells, plants, seeds or products described herein may comprise one or more of the following nucleic acid or amino acid sequences, or a portion of one or more of the disclosed sequences. Non-limiting examples of target polynucleotides are set forth below in Tables 1, 2, and 3, and SEQ ID NOs: 1-86, or variants or fragments thereof, or complements thereof. The official copy of the sequence listing is submitted with the specification as a text file via EFS-Web, in compliance with the American Standard Code for Information Interchange (ASCII). The sequence listing filed via EFS-Web is part of the specification and is hereby incorporated in its entirety by reference herein, and comprises SEQ ID. NOs.: 1-86.

TABLE 1
Sample Source and Fragment Information.

| Target | | | Seq | Length |
|---|---|---|---|---|
| Name | Initial SEQ ID | Frag GENE ID | No. | (bp) |
| DV-RYANR- | idv1c.pk035.i17.f:fis | ryanodine receptor-like | 1 | 211 |
| FRAG1 | | protein | | |
| DV-HP2- | idv1c.pk037.j20.f | hypothetical protein | 2 | 162 |
| FRAG7 | | | | |
| DV-PAT3- | idv1c.pk002.j17.f:fis | proteosome subunit | 3 | 161 |
| FRAG13 | | alpha type 3 | | |
| DV-PROTB- | idv1c.pk003.d6.f:fis | Proteosome subunit | 4 | 203 |
| FRAG1 | | beta type 1 | | |
| DV-RPS10- | idv1c.pk001.e9.f:fis | ribosomal protein | 5 | 557 |
| FRAG4 | | s10e | | |

TABLE 2
Ssk Homologues.

| | | | | | Tran- |
|---|---|---|---|---|---|
| | Common | | ORF | Tran- | script |
| Sources | name | ORF* | Length† | script* | Length† |
| Spodoptera | Fall Army | 6 | 474 | 36 | 1147 |
| frugiperda | Worm | | | | |
| Ostrinia nubilalis | European | 7 | 474 | 37 | 1482 |
| | Corn Borer | | | | |
| Helicoverpa zea | Corn Earworm | 8 | 474 | 38 | 1035 |
| Phyllotreta | crucifer flea | 9 | 480 | 39 | 1564 |
| cruciferae | beetle | | | | |
| Phyllotreta | striped flea | 10 | 480 | 40 | 1212 |
| striolata | beetle | | | | |
| Acyrthosiphon | pea aphid | 11 | 483 | 41 | 1375 |
| pisum | | | | | |
| Apis florea | dwarf honey | 12 | 483 | 42 | 645 |
| | bee | | | | |
| Apis mellifera | western honey | 13 | 489 | 43 | 683 |
| | bee | | | | |
| Bombus impatiens | eastern | 14 | 489 | 44 | 2177 |
| | bumble bee | | | | |
| Bombus terrestris | buff-tailed | 15 | 489 | 45 | 2156 |
| | bumblebee | | | | |
| Nasonia | jewel wasps | 16 | 480 | 46 | 1430 |
| vitripennis | | | | | |
| Megachile | alfalfa | 17 | 492 | 47 | 738 |
| rotundata | leafcutter bee | | | | |
| Spodoptera exigua | Beet | 18 | 474 | 48 | 1052 |
| | Armyworm | | | | |
| Chrysopa pallens | living | 19 | 477 | 49 | 1079 |
| | lacewing | | | | |
| | insect | | | | |
| Bombyx moi | silkworm | 20 | 474 | 50 | 1380 |
| Phlebotomus | sandfly | 21 | 474 | 51 | 700 |
| papatasi | | | | | |
| Culex | southern house | 22 | 450 | 52 | 450 |
| quinquefasciatus | mosquito | | | | |
| Anopheles | mosquitoes | 23 | 474 | 53 | 951 |
| gambiae | | | | | |
| Aedes aegypti | yellow fever | 24 | 441 | 54 | 939 |
| | mosquito | | | | |
| Dendroctonus | mountain pine | 25 | 477 | 55 | 1002 |
| ponderosae | beetle | | | | |
| Pediculus | Human lice | 26 | 468 | 56 | 468 |
| humanus corporis | | | | | |
| Nezara viridula | Southern | 27 | 480 | 57 | 635 |
| | Green Stink | | | | |
| | Bug | | | | |
| Lygus hesperus | Western Plant | 28 | 483 | 58 | 1080 |
| | Bug | | | | |
| Euschistus servus | Brown Stink | 29 | 480 | 59 | 1237 |
| (Say) | Bug | | | | |
| Halyomorpha | Brown | 30 | 405 | 60 | 874 |
| halys | marmorated | | | | |
| | stink bug | | | | |
| Megacopta | Kudzu Bug | 31 | 480 | 61 | 1263 |
| cribraria | | | | | |
| Manduca sexta | Tobacco | 32 | 474 | 62 | 1445 |
| | hornworm | | | | |
| Euschistus heros | Neotropical | 33 | 480 | 63 | 880 |
| | Brown Stink | | | | |
| | Bug | | | | |
| Piezodorus | Red-banded | 34 | 480 | 64 | 1243 |
| guildinii | Stink Bug | | | | |
| Tribolium | red flour | 35 | 477 | 65 | 797 |
| castaneum | beetle | | | | |

*The number given in the column for ″ORF″ or ″Transcript,″ as indicated, is the SEQ ID NO corresponding to the Ssk homologue. ″ORF″ is the coding region or open reading frame fragment for protein encoded by the given Ssk homologue. ″Transcript″ corresponds to the full-length mRNA transcript for the given Ssk homologue.
†Length in base pairs (bp).

TABLE 3
HP2 Homologues.

| | | | | | Tran- |
|---|---|---|---|---|---|
| | Common | | ORF | Tran- | script |
| Sources | name | ORF* | Length† | script* | Length† |
| Diabrotica | Western corn | 66 | 3948 | 77 | 4364 |
| virgifera virgifera | rootworm | | | | |
| Diabrotica | Southern Corn | 67 | 3771 | 78 | 5238 |
| undecimpunctata | Rootworm | | | | |
| howardi | | | | | |
| Diabrotica | Northern Corn | 68 | 4068 | 79 | 4313 |
| barberi | Rootworm | | | | |
| Leptinotarsa | Colorado | 69 | 3843 | 80 | 5020 |
| decemlineata | potato beetle | | | | |
| Helicoverpa zea | Corn Earworm | 70 | 4455 | 81 | 4851 |
| Spodoptera | Fall Army | 71 | 4473 | 82 | 7269 |
| frugiperda | Worm | | | | |
| Ostrinia | European | 72 | 4494 | 83 | 5316 |
| nubilalis | Corn Borer | | | | |
| Phyllotreta | crucifer flea | 73 | 3807 | 84 | 4330 |
| cruciferae | beetle | | | | |
| Phyllotreta | striped flea | 74 | 3837 | 85 | 4302 |
| striolata | beetle | | | | |
| Megacopta | kudzu bug | 75 | 3429 | 86 | 3847 |
| cribraria | | | | | |
| Tribolium | Flour beetles | 76 | 4287 | — | — |
| castaneum | | | | | |

*The number given in the column for ″ORF″ or ″Transcript,″ as indicated, is the SEQ ID NO corresponding to the Ssk homologue. ″ORF″ is the coding region or open reading frame fragment for protein encoded by the given Ssk homologue. ″Transcript″ corresponds to the full-length mRNA transcript for the given Ssk homologue.
†Length in base pairs (bp).

### Example 2: In Vitro Transcript dsRNA

Two previously identified RNAi active targets RYANR and HP2 showed 54% and 49% identity to Drosophila snakeskin (Ssk) and Mesh, respectively. Without wishing to be bound by a particular theory, it is hypothesized that the Mesh-Ssk protein complex is required for septate junction formation in the Drosophila midgut.

Regions (fragments) of WCRW Ssk genes were produced by PCR using target specific primers to generate DNA templates and, followed by in vitro transcription (IVT) to produce long double stranded RNAs. The target specific primers also contain T7 RNA polymerase sites (T7 sequence at 5′ end of each primer). Following enzymatic digestion and removal of the DNA template, the IVT reaction products were incorporated into artificial insect diet as described below. Briefly, five double stranded RNA (dsRNA) samples were used in two tests for the LT₅₀ comparisons in three tests (See Table 4 below). RYANR-Frag 1 and four other samples (HP2-7, PAT3-13, ProtB-1 and RPS10-4) were used in Test 1 and 2. RYANR-Frag 1. The dsRNA samples were produced using Ambion Megascript® High Yield Transcription Kit from the PCR product. The PCR product was obtained using gene specific primers with the T7 promoter region and gene- and species-specific template DNA. A gel was run to check for yield and product size. The samples were purified using Ambion Megaclear® kit. Samples were diluted 1:200 and quantified by absorbance at 260 nm using laboratory spectrophotometer.

TABLE 4
dsRNA Sample List.

| Sample # | Test 1, 2 |
|---|---|
| 1 | DV-HP2-FRAG7 |
| 2 | DV-PAT3-FRAG13 |
| 3 | DV-PROTB-FRAG1 |
| 4 | DV-RPS10-FRAG4 |
| 5 | DV-RYANR-FRAG1 |

### Example 3: Larval/Neonate LT₅₀ Assay

The LT50 assay requires a 2-step procedure in an artificial diet assay as follows:

Step 1: Larvae are fed for 24 hours on an artificial WCRW diet prepared in 96 well plates. In Test 1, the sample portion of the incorporated diet is prepared using 5 ul of distilled water and 25 μl of diet, while in Test 2, a 5 μl of a dsRNA sample (at 300 ppm=μg/ml) was incorporated into 25 μl of diet (at 50 ppm for sample-diet mixture). Distilled water was used for sample dilutions and as a negative control in each test. WCRW neonates are washed out of hatch plates and set aside in escape proof containers. Multiple WCRW neonates were infested per well using a drop plate infestation method. Plates are placed in a zip-lock bag and incubated for 24 hours in a growth chamber set at 27±1° C. and 65+5% RH.

Step 2: 48 well flat bottom plates were used for the LT₅₀ tests. A WCRW diet was prepared and added to each tube using a sterile syringe to reach 5 ppm for Test 1 or 50 ppm for Test 2, after thoroughly mixing each dsRNA sample solution and diet at a ratio of 1:5. Distilled water was used for sample dilutions and as a negative control in each test. The diet fed WCRW larvae from Step 1, Test 1 without dsRNA treatment and Test 2 with dsRNA treatment, were tapped out of the 96 well plates onto sterile paper. A single larva was infested into each well of a corresponding 48 well plate for each sample using a sterile pipe cleaner or paint brush.

The 48 well plates for Step 2 of each test were placed in a growth chamber set at 27±1° C. and 65+5% RH. Each plate was checked daily for mortality 1-12 days after infestation in Step 1 (Test 2) or Step 2 (Test 1). Weibull distribution for Survival analysis in SAS JMP (Version 11.0) was used to describe the time to death curve. Each insect is treated as an individual data point for the LT₅₀ output based on Weibull distribution. LT₅₀ values are significantly different if non-overlap of 95% CI (P<0.05).

To determine the LT₅₀ for other sequences or other insects, the assay format of Test 1 is used, not the assay format of Test 2 (feeding RNA construct from day 1). The LT₅₀ assay, as described for Test 1, can be used to determine rapid acting sequences for pest control in any pest insect species, that is, those RNA sequences that have an LT₅₀ that is less than 80% of the average LT₅₀ determined wherein, from the same insect, the average LT₅₀ is determined using each nucleotide sequence encoding a homologous protein comprising at least 45% amino acid identity to the protein encoded by each of SEQ ID NOs.: 5, 7, and 9. For example, for a particular insect, it is contemplated that there would be three nucleotide sequences for determining the average LT₅₀ in an LT₅₀ assay, and each of those three nucleotide would encode a homologous protein comprising at least 45% amino acid identity for each of SEQ ID NOs.: 5, 7, and 9.

For example, for corn rootworms, a rapid acting sequence is determined for an RNA construct where the LT₅₀ is less than 80% of the average LT₅₀ determined using SEQ ID NOs.: 5, 7, and 9. For example, from an insect, a dsRNA construct of at least a portion of a gene that is homologous to RYANR or HP-2 is provided for testing and its LT₅₀ is determined and if the LT₅₀ is less than 80% of the average LT₅₀, the dsRNA construct is a fast acting construct for pest control. The average LT₅₀ is determined using, from the same insect, each nucleotide sequence encoding a homologous protein comprising at least 45% amino acid identity to the protein encoded by each of SEQ ID NOs.: 5, 7, and 9

The LT₅₀ is determined using a larval/neonate LT50 assay comprising, a first feeding step wherein insect larvae are fed for 24 hours an artificial insect diet comprising without the test RNA construct; and, a second feeding step wherein the insect larvae are fed for the following eleven days an artificial insect diet comprising the test RNA construct; and determining the time to death curve for the insect in the assay from calculation of a Weibull distribution for survival analysis.

The amount of dsRNA used in an LT₅₀ assay may be determined by routine experimentation to define a LT₅₀ that is less than 80% of the average LT₅₀ determined using SEQ ID NOs: 5, 7 and 9 or homologs of SEQ ID NOs.: 5, 7, and 9, depending on the insect. For example, the amount of dsRNA used may be from about 10 ppm=μg/ml to about 1000 ppm=μg/ml, or more, or about 10 ppm=μg/ml, about 20 ppm=μg/ml, about 30 ppm=μg/ml, about 50 ppm=μg/ml, about 60 ppm=μg/ml, about 70 ppm=μg/ml, about 80 ppm=μg/ml, about 100 ppm=μg/ml, about 150 ppm=μg/ml, about 180 ppm=μg/ml, about 200 ppm=μg/ml, about 250 ppm=μg/ml, about 300 ppm=μg/ml, about 350 ppm=μg/ml, about 400 ppm=μg/ml, about 450 ppm=μg/ml, about 500 ppm=μg/ml, about 550 ppm=μg/ml, about 600 ppm=μg/ml, about 650 ppm=μg/ml, about 700 ppm=μg/ml, about 750 ppm=μg/ml, about 800 ppm=μg/ml, about 850 ppm=μg/ml, about 900 ppm=μg/ml, about 950 ppm=μg/ml, about 1000 ppm=μg/ml, or more.

### Example 4. Results of Larval/Neonate LT50 Assay

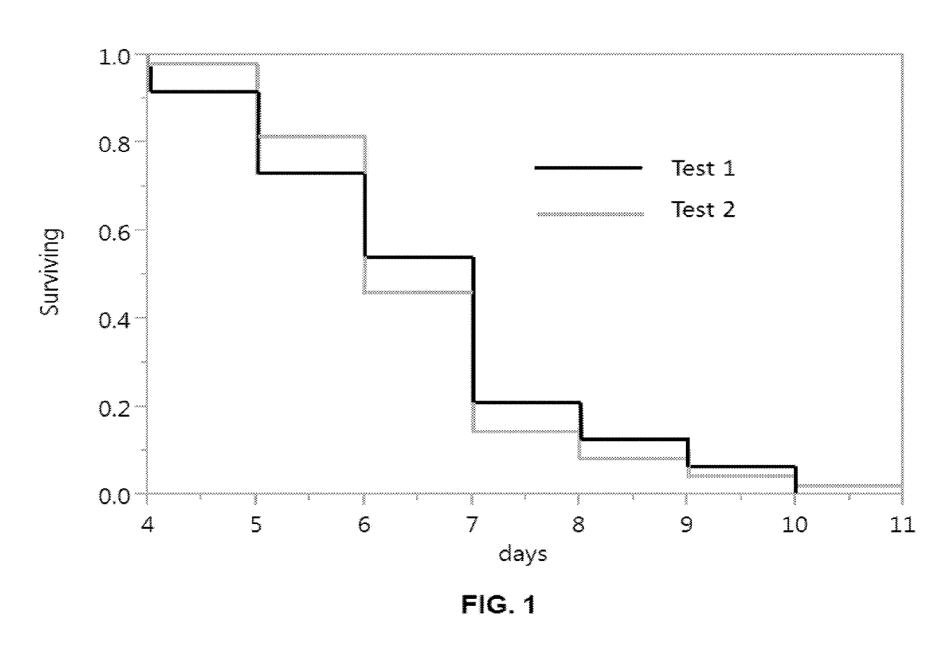

The LT₅₀ of RYANR-Frag 1 was 6.6 d in Test 1 (P=0.72, Log-Rank test, see Table 5 below and FIG. 1). The data from Test 1 with RYANR-Frag 1 as the positive control. RYANR-Frag 1 had the lowest LT50 (6.6 d) with LT₅₀<7 d at 5 ppm treatment using one day old diet-fed WCRW larvae. RYANR-Frag1 was in the same group as HP2-Frag 7 (7.1 d) and both had significantly lower LT₅₀ than other samples.

TABLE 5
LT₅₀ results of RYANR-Frag 1.*

| | | LT50 | Lower | Upper | |
|---|---|---|---|---|---|
| Sample | Test | (d) | 95% CI | 95% CL | Grouping** |
| DV-RYANR- | 1 | 6.6 | 6.2 | 7.1 | a |
| FRAG1 | | | | | |

*Tested at 5 ppm in Test 1 on WCRW (n = 48).
**P = 0.72, Log-Rank test.

In order to further assess the dsRNAs, Test 2 was repeated at a higher dose (50 ppm) in the diet plug. In this experiment, RYANR-Frag 1 had lowest LT₅₀ (5.5 d) at 50 ppm treatment by two-step exposures, one for WCRW neonates and the other for one day old larvae at the same dose (see Table 6 below). RYANR-Frag 1 had significantly lower LT50 than other 4 samples (HP2-7, PAT3-13, ProtB-1 and RPS10-4) with LT50>6 d. The results indicated that RYANR-Frag 1 had lower LT₅₀ on WCRW than the other samples examined, suggesting fast insect control activity on either neonates or one day old died fed larvae. The LT₅₀ of RYANR is in the same group as HP2 in Test 1 at 5 ppm but significantly lower than HP2 in Test 2 at 50 ppm.

TABLE 6
LT₅₀ results of 5 dsRNA samples.*

| | | 12d | | | |
|---|---|---|---|---|---|
| | 12d % | LT50 | Lower | Upper | |
| Sample | mortality | (d) | 95% CI | 95% CI | Grouping** |
| DV-HP2-FRAG7 | 100 | 6.3 | 6 | 6.7 | b |
| DV-PAT3-FRAG13 | 93.5 | 9.0 | 8.4 | 9.7 | c |
| DV-PROTB-FRAG1 | 97.8 | 8.5 | 7.9 | 9 | c |
| DV-RPS10-FRAG4 | 97.8 | 9.0 | 8.5 | 9.7 | c |
| DV-RYANR-FRAG1 | 100 | 5.5 | 5.2 | 5.8 | a |

*Tested at 50 ppm in Test 2 on WCRW (n = 45-48).
**LT50s with different letters are significantly different based on non-overlap of 95% CI (P < 0.05).

### Example 5. Insecticidal Activity Against WCRW Adult in Diet Assay

The assay was carried out for 11 days with diet plugs refreshed daily. A total of 16 beetles (2-3 days old from insectary) were used for each of following four treatments: (1) RYANR frag1 dsRNA, (2) GUS dsRNA, (3) Buffer (elution buffer, Qiagen) and (4) sterile water.

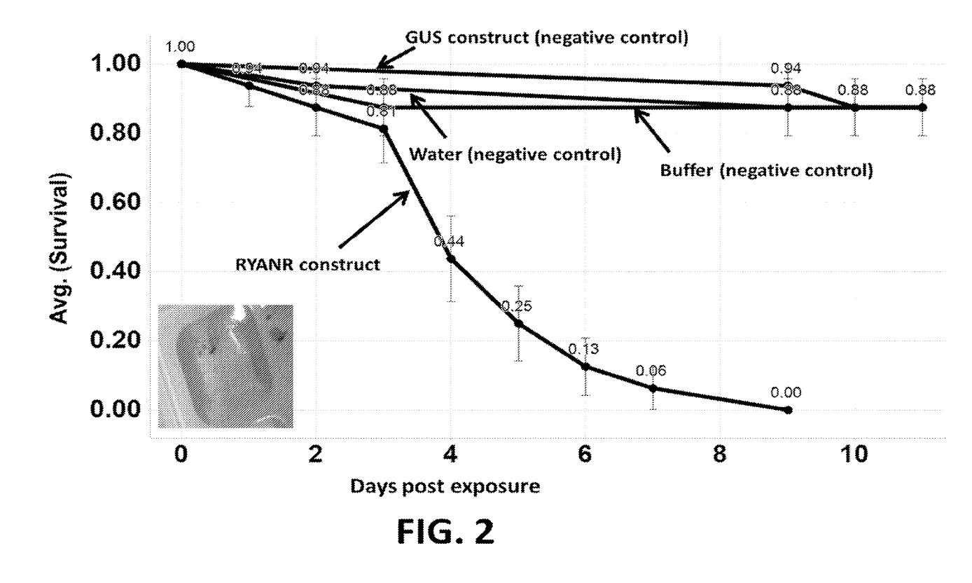

25 μl RNA sample or control/well in the 96 well plate, then add 75 μl per well of prepared media in 96-well tray. The 96-well trays were stored in refrigerator until used. Mortality was recorded on a daily basis. Survival analysis was used to generate the median survival time (LT₅₀) and the homogeneity of the survival curve was compared using Kaplan Meier Log Rank test (JMP). At 100 ppm, RYANR (DV-RYANR-FRAG1, SEQ ID NO.: 1) killed about 50% adults in average 4.56 days and showed significant insecticidal activities compared to GUS dsRNA, buffer and water controls (FIG. 2).

### Example 6. Agrobacterium-Mediated Transformation of Maize

For Agrobacterium-mediated maize transformation with the disclosed polynucleotide constructs comprising a silencing element as disclosed herein, the method of Zhao can be employed (U.S. Pat. No. 5,981,840 and International Patent Publication Number WO 1998/32326, the contents of which are hereby incorporated by reference). Briefly, immature embryos are isolated from maize and the embryos are contacted with an Agrobacterium suspension, where the bacteria are capable of transferring the desired disclosed polynucleotide constructs comprising a silencing element as disclosed herein to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional resting step can be contemplated. In this resting step, the embryos are incubated in the presence of at least one antibiotic known to inhibit Agrobacterium growth without a plant transformant selective agent (step 3: resting step). The immature embryos are cultured on solid medium with antibiotic, but without a selecting agent, for Agrobacterium elimination and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus can then be regenerated into plants (step 5: the regeneration step), and calli grown on selective medium are cultured on solid medium to regenerate the plants.

### Example 7. Expression of Silencing Elements in Maize

The silencing elements can be expressed in a maize plant as hairpins using the transformation techniques described herein above in Example 7, and the plant will test for insecticidal activity against corn root worms.

Maize plants are transformed with plasmids containing genes listed in Table 1 or 2, and plants expressing the silencing elements are transplanted from 272V plates into greenhouse flats containing Fafard Superfine potting mix. Approximately 10 to 14 days after transplant, plants (now at growth stage V2-V3) are transplanted into treepots containing Fafard Superfine potting mix. At 14 days post greenhouse send date, plants are infested with 200 eggs of Western Corn Rootworms (WCRW)/plant. For later sets, a second infestation of 200 eggs WCRW/plant can be done 7 days after the first infestation and scoring is at 14 days after the second infestation. 21 days post infestation, plants are scored using CRWNIS. Those plants with a score of <0.5 are transplanted into large pots containing SB300 for T1 seed.

The sequences referred to herein, SEQ. ID NOs: 1-86, are filed concurrently herewith in a textfile and are incorporated herein in their entirety.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

That which is claimed:

1. An RNA construct comprising at least one double-stranded RNA region, at least one strand of which that is complementary to a polynucleotide encoding a protein comprising at least 45% amino acid identity to the protein encoded by SEQ ID NOs.: 1 or 71, or variants or fragments thereof, or complements thereof;
wherein the RNA construct has insecticidal activity against a plant insect pest; and
wherein the RNA construct has an LT₅₀ that is less than 80% of the average LT₅₀, wherein the LT₅₀ is determined using SEQ ID NOs.: 5, 7, and 9, or wherein the average LT₅₀ is determined using each nucleotide sequence encoding a homologous protein comprising at least 45% amino acid identity to the protein as encoded by each of SEQ ID NOs.: 5, 7, and 9;
wherein the LT₅₀ is determined using a larval/neonate LT₅₀ assay comprising,
a first feeding step wherein insect larvae are fed for 24 hours an artificial insect diet comprising without the test RNA construct; and
a second feeding step wherein the insect larvae are fed for the following eleven days an artificial insect diet comprising the test RNA construct; and
determining the time to death curve for the larvae in the assay from calculation of a Weibull distribution for survival analysis.

2. The RNA construct of claim 1, wherein the at least one double-stranded RNA region comprises a polynucleotide that is complementary to:
(a) a nucleotide sequence comprising any one of SEQ ID NOs.: 1-86, or variants and fragments thereof, and complements thereof;
(b) a nucleotide sequence comprising at least 90% sequence identity to any one of nucleotides SEQ ID NOs.: 1-86, or variants and fragments thereof, and complements thereof, wherein the polynucleotide encodes a silencing element having insecticidal activity against a plant insect pest; or
(c) the nucleotide sequence comprising at least 19 consecutive nucleotides of any one of SEQ ID NOs.: 1-86, or variants and fragments thereof.

3. The RNA construct of claim 1, wherein the RNA construct has an LT₅₀ that is less than 70% of the average LT₅₀ determined using SEQ ID NOs.: 5, 7, and 9, or sequences homologous to SEQ ID NOs.: 5, 7, and 9.

4. The RNA construct of claim 1, wherein the plant insect pest comprises a Coleoptera plant pest.

5. The RNA construct of claim 4, wherein the Coleoptera plant pest comprises a Diabrotica plant pest.

6.-8. (canceled)

9. The RNA construct of claim 1, wherein the RNA construct comprises a hairpin loop.

10. The RNA construct of claim 1, wherein the RNA construct comprises a first segment, a second segment, and a third segment, wherein
a. the first segment comprises at least 19 consecutive nucleotides having at least 90% sequence complementarity to a target sequence set forth in any one of SEQ ID NOs.: 1-86, or variants and fragments thereof, and complements thereof;
b. the second segment comprises a loop of sufficient length to allow the RNA construct to be transcribed as a hairpin RNA; and,
c. the third segment comprises at least 19 consecutive nucleotides having at least 85% complementarity to the first segment.

11. A DNA construct comprising a polynucleotide encoding the RNA construct of claim 1.

12. An expression construct comprising a DNA construct of claim 11.

13. The expression construct of claim 12, wherein the polynucleotide is operably linked to a heterologous promoter.

14. The expression construct of claim 12, wherein the polynucleotide is flanked by a first operably linked convergent promoter at one terminus of the polynucleotide and a second operably linked convergent promoter at the opposing terminus of the polynucleotide, wherein the first and the second convergent promoters are capable of driving expression of the RNA construct.

15. A host cell comprising the the expression construct of claim 12.

16. The host cell of claim 15, wherein the host cell is a bacterial cell.

17.-26. (canceled)

27. A plant cell having stably incorporated into its genome a heterologous polynucleotide encoding a double stranded RNA, wherein the polynucleotide comprises
a. a fragment of at least 19 consecutive nucleotides of any one SEQ ID NOs.: 1-86, or variants and fragments thereof, and complements thereof; or,
b. the nucleotide sequence comprising at least 90% sequence identity to any one of SEQ ID NOs.: 1-86, or variants and fragments thereof;
wherein the double stranded RNA controls the insect pest.

28. The plant cell of claim 27, wherein the plant insect pest comprises a Coleoptera plant pest

29. The plant cell of claim 27, wherein the Coleoptera plant pest comprises a Diabrotica plant pest.

30.-32. (canceled)

33. The plant cell of claim 27, wherein the double stranded RNA comprises a nucleotide sequence complementary to:
a. a polynucleotide comprising the sequence set forth in any one of SEQ ID NOs.: 1-86, or variants and fragments thereof, and complements thereof; or
b. a polynucleotide comprising at least 130 consecutive nucleotides of the sequence set forth in any one SEQ ID NOs.: 1-86, or variants and fragments thereof, and complements thereof.

34.-37. (canceled)

38. The plant cell of claim 27, wherein the polynucleotide is operably linked to a heterologous promoter.

39. The plant cell of claim 27, wherein the plant cell is from a monocot or a dicot.

40.-42. (canceled)

43. A plant or plant part comprising the plant cell of claim 27.

44. A transgenic seed from the plant of claim 43.

45. A method for controlling a plant pest comprising feeding to a plant pest a composition comprising
an RNA construct comprising at least one double-stranded RNA region, at least one strand of which is complementary to a polynucleotide encoding a protein comprising at least 45% amino acid identity to the protein encoded by SEQ ID NOs.: 1 or 71, or variants or fragments thereof, or complements thereof;
wherein the RNA construct has insecticidal activity against a plant insect pest;
wherein the RNA construct has an LT₅₀ that is less than 80% of the average LT₅₀, wherein the LT₅₀ is determined using SEQ ID NOs.: 5, 7, and 9, or wherein the average LT₅₀ is determined using each nucleotide sequence encoding a homologous protein comprising at least 45% amino acid identity to the protein as encoded by each of SEQ ID NOs.: 5, 7, and 9, and
wherein the LT₅₀ is determined using a larval/neonate LT₅₀ assay comprising:
a) a first feeding step wherein insect larvae are fed for 24 hours an artificial insect diet comprising without the test RNA construct; and
b) a second feeding step wherein the insect larvae are fed for the following eleven days an artificial insect diet comprising the test RNA construct; and
c) determining the time to death curve for the larvae in the assay from calculation of a Weibull distribution for survival analysis.

46. The method of claim 45, wherein the plant pest comprises a Coleoptera plant pest.

47. The method of claim 46, wherein the Coleoptera plant pest comprises a Diabrotica plant pest.

48.-96. (canceled)
